(19) United States
(12) Patent Application Publication
(10) Pub. No.: US 2023/0233475 A1
(43) Pub. Date: Jul. 27, 2023

(54) LIPID NANOPARTICLES CONTAINING POLYNUCLEOTIDES ENCODING GLUCOSE-6-PHOSPHATASE AND USES THEREOF

(71) Applicant: ModernaTX, Inc., Cambridge, MA (US)

(72) Inventors: Kerry Benenato, Sudbury, MA (US); Kristine Burke, South Boston, MA (US); Jingsong Cao, West Roxbury, MA (US); Paloma Hoban Giangrande, Belmont, MA (US); Edward J. Hennessy, Westwood, MA (US); Stephen Hoge, Brookline, MA (US); Jaclyn Milton, Somerville, MA (US); Staci Sabnis, Medford, MA (US); Timothy Salerno, Somerville, MA (US); Matthew Theisen, Wakefield, MA (US)

(21) Appl. No.: 17/928,684

(22) PCT Filed: Jun. 1, 2021
(86) PCT No.: PCT/US2021/035197

§ 371 Date: Nov. 30, 2022

Related U.S. Application Data
(60) Provisional application No. 63/032,923, filed on Jun. 1, 2020.
(60) Provisional application No. 63/073,657, filed on Sep. 2, 2020.

(51) Int. Cl. A61K 9/51; A61K 31/7088; A61K 9/00; C12N 9/16; A61P 35/00

(52) U.S. Cl. CPC A61K 9/5123; A61K 31/7088; A61K 9/0019; C12Y 301/03009; C12N 9/16; A61P 35/00

(57) ABSTRACT

This disclosure relates to ionizable lipid-based lipid nanoparticles for delivery of mRNA encoding glucose-6-phosphatase. Lipid nanoparticle/mRNA therapies of the invention increase and/or restore deficient levels of glucose-6-phosphatase expression and activity in subjects and are useful for the treatment of glycogen storage disease type 1a (GSD-Ia). Lipid nanoparticle/mRNA therapies of the invention increase glucose production and reduce the abnormal accumulation of glycogen and glucose-6-phosphate associated with GSD-Ia.

### CROSS-REFERENCE TO RELATED APPLICATIONS

This application claims the priority benefit of U.S. Provisional Application No. 63/032,923, filed Jun. 1, 2020, and U.S. Provisional Application No. 63/073,657, filed Sep. 2, 2020, the contents of each which is incorporated by reference in its entirety herein.

### SEQUENCE LISTING

The instant application contains a Sequence Listing which has been submitted electronically in ASCII format and is hereby incorporated by reference in its entirety. Said ASCII copy, created on Jun. 1, 2021, is named 45817-0090WO1_SL.txt and is 60,999 bytes in size.

### BACKGROUND

Glycogen storage disease type 1 (GSD-I), also known as von Gierke disease, is caused by an accumulation of glycogen in organs and tissues of the body. GSD-I can be divided into two major subtypes, including glycogen storage disease type 1a (GSD-Ia, MIM232200) and type Ib (GSD-Ib, MIM232220). Chou et al., Liver Research, 2017, 1:174-180. GSD-I has an incidence of about 1 in 100,000. GSD-Ia is an autosomal recessive metabolic disorder that is caused by a deficiency in glucose-6-phosphatase (G6Pase or G6PC; EC 3.1.3.9), and accounts for about 80% of GSD-I cases. G6PC is a key enzyme that is necessary for glucose production that is expressed mostly in the liver, kidney, and intestine, and functions inside of the endoplasmic reticulum (ER) lumen of cells. G6PC catalyzes the hydrolysis of glucose-6-phosphate (G6P) to glucose and phosphate late during gluconeogenesis and glycogenolysis. Normally, as blood glucose levels fall in between meals, G6P is produced during the terminal step of gluconeogenesis and glycogenolysis in the gluconeogenic organs (primarily liver, and also the kidneys and intestine) and it is hydrolyzed by G6PC to glucose and then released back into the blood.

GSD-Ia is caused by mutation of the gene encoding G6PC that impairs the enzyme's ability to hydrolyze G6P. After a short fast of a few hours, individuals with GSD-Ia become hypoglycemic. G6P becomes elevated in cellular cytoplasm, which leads to an accumulation of glycogen and fat in the liver and kidneys, and impaired blood glucose homeostasis. The accumulation of glycogen promotes progressive hepatomegaly and nephromegaly. GSD-Ia patients can also develop other metabolic complications, including hypercholesterolemia, hypertriglyceridemia, hyperuricemia, hyperlipidemia, and lactic academia. Individuals with GSD-Ia can also develop longer term problems, including growth retardation, osteoporosis, gout, pulmonary hypertension, and renal disease. One long-term complication is the development of hepatic tumors in 75% of GSD-Ia patients over the age of 25 years old, of which, approximately 10% transform into malignant tumors.

### SUMMARY

The present disclosure provides ionizable lipid-based lipid nanoparticles for delivery of messenger RNA (mRNA) encoding glucose-6-phosphatase in vivo. The lipid nanoparticle/mRNA therapeutics of the invention are particularly well-suited for the treatment of GSD-Ia, as the technology provides for the intracellular delivery of mRNA encoding glucose-6-phosphatase followed by de novo synthesis of functional glucose-6-phosphatase protein within target cells.

In one aspect, the disclosure features a lipid nanoparticle comprising:

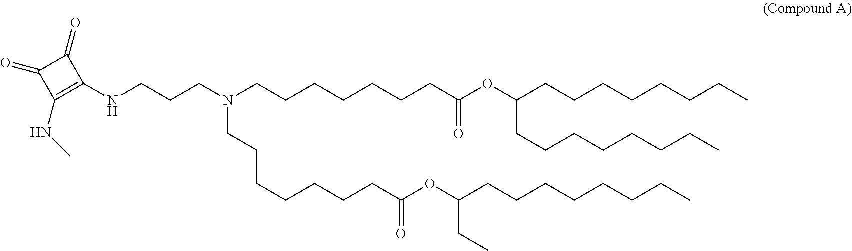 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or its N-oxide, or a salt or isomer thereof; and

a mRNA comprising an open reading frame (ORF) encoding the glucose phosphatase polypeptide of SEQ ID NO:1, wherein the ORF comprises the nucleic acid sequence of SEQ ID NO:2.

In some embodiments, the mRNA comprises a 5′ UTR comprising the nucleic acid sequence of SEQ ID NO:55.

In some embodiments, the mRNA comprises a 5′ UTR comprising a nucleic acid sequence depicted in Table 1.

In some embodiments, the mRNA comprises a 3′ UTR comprising the nucleic acid sequence of SEQ ID NO:114.

In some embodiments, the mRNA comprises a 3′ UTR comprising a nucleic acid sequence depicted in Table 2 or 3.

In some embodiments, the mRNA comprises a 5′ UTR comprising a nucleic acid sequence depicted in Table 1 and a 3′ UTR comprising a nucleic acid sequence depicted in Table 2 or 3.

In some embodiments, the mRNA comprises the nucleic acid sequence of SEQ ID NO:5.

In some embodiments, the mRNA comprises a 5′ terminal cap (e.g., a guanine cap nucleotide containing an N7 methylation and the 5′-terminal nucleotide of the mRNA contains a 2′-O-methyl).

In some embodiments, the mRNA comprises a poly-A region (e.g., at least about 100 nucleotides in length).

In some embodiments, all of the uracils of the mRNA are N1-methylpseudouracil s.

In some embodiments, the mRNA comprises a 5′ terminal cap comprising a guanine cap nucleotide containing an N7 methylation and the 5′-terminal nucleotide of the mRNA contains a 2′-O-methyl, the mRNA comprises the nucleotide sequence of SEQ ID NO:5, the mRNA comprises a poly-A region at least about 100 nucleotides in length, and all of the uracils of the mRNA are N1-methylpseudouracils.

In some embodiments, the lipid nanoparticle comprises Compound I.

In some embodiments, the lipid nanoparticle comprises a phospholipid and a structural lipid.

In some embodiments, the lipid nanoparticle comprises: (a) cholesterol and Compound I; (b) cholesterol and PEG-DMG; (c) DSPC, cholesterol, and Compound I; (d) DOPE, cholesterol, and Compound I; (e) DSPC, cholesterol, and PEG-DMG; or (f) DOPE, cholesterol, and PEG-DMG.

In another aspect, the disclosure features a method of expressing a glucose-6-phosphatase polypeptide in a human subject in need thereof, comprising administering to the human subject an effective amount of a lipid nanoparticle disclosed herein.

In another aspect, the disclosure features a method of treating, preventing, or delaying the onset and/or progression of GSD-Ia in a human subject in need thereof, comprising administering to the human subject an effective amount of a lipid nanoparticle disclosed herein.

In another aspect, the disclosure features a method of increasing blood, plasma, and/or serum glucose levels in a human subject in need thereof, comprising administering to the human subject an effective amount of a lipid nanoparticle disclosed herein.

In another aspect, the disclosure features a method of reducing liver glycogen levels in a human subject in need thereof, comprising administering to the human subject an effective amount of a lipid nanoparticle disclosed herein.

In another aspect, the disclosure features a method of reducing liver G6P levels in a human subject in need thereof, comprising administering to the human subject an effective amount of a lipid nanoparticle disclosed herein.

In another aspect, the disclosure features a method of reducing serum and/or liver triglyceride levels in a human subject in need thereof, comprising administering to the human subject an effective amount of a lipid nanoparticle disclosed herein.

In another aspect, the disclosure features a method of increasing G6PC activity (e.g., in liver and/or blood) in a human subject in need thereof, comprising administering to the human subject an effective amount of a lipid nanoparticle disclosed herein.

In another aspect, the disclosure features a method of treating or preventing liver adenoma (e.g., hepatocellular adenoma) in a human subject having glycogen storage disease type 1a (GSD-Ia), comprising administering to the human subject an effective amount of a lipid nanoparticle disclosed herein.

In another aspect, the disclosure features a method of treating or preventing liver carcinoma (e.g., hepatocellular carcinoma) in a human subject having glycogen storage disease type 1a (GSD-Ia), comprising administering to the human subject an effective amount of a lipid nanoparticle disclosed herein.

In another aspect, the disclosure features a method of treating a liver tumor in a human subject having glycogen storage disease type 1a (GSD-Ia), comprising administering to the human subject an effective amount of a lipid nanoparticle disclosed herein.

In another aspect, the disclosure features a method of preventing liver tumor formation in a human subject having glycogen storage disease type 1a (GSD-Ia), comprising administering to the human subject an effective amount of a lipid nanoparticle disclosed herein.

In some embodiments of any of the foregoing methods, the human subject has been fasting (e.g., for at least 2 hours).

In some embodiments of any of the foregoing methods, the method entails multiple administrations of the lipid nanoparticle to the human subject (e.g., at intervals of about once a week, about once every two weeks, or about once a month).

In some embodiments of any of the foregoing methods, the mRNA is administered at a dose of 0.1 mg mRNA/kg of body weight of the human subject.

In some embodiments of any of the foregoing methods, the mRNA is administered at a dose of 0.2 mg mRNA/kg of body weight of the human subject.

In some embodiments of any of the foregoing methods, the mRNA is administered at a dose of 0.5 mg mRNA/kg of body weight of the human subject.

In some embodiments of any of the foregoing methods, the lipid nanoparticle is administered intravenously.

### BRIEF DESCRIPTION OF THE DRAWINGS

FIG. 1 is a graph showing fasting blood glucose levels in L-G6PC(−/−) mice injected with 0.1 mg/kg, 0.2 mg/kg, or 0.5 mg/kg of mRNA (SEQ ID NO:5) in lipid nanoparticles. Wild-type mice were injected with Tris-sucrose. Control L-G6PC(−/−) mice were injected with Tris-sucrose or mRNA encoding eGFP (0.5 mg/kg). The dotted horizontal line denotes the threshold blood glucose level to maintain normal physiology. The mRNA treatments are depicted with square boxes: of the three mRNA treatments, 0.5 mg/kg is the top line from days 1 to 10, 0.2 mg/kg is the middle line from days 1 to 10, and 0.1 mg/kg is the bottom line from days 1 to 10.

FIG. 2A is a bar graph showing 6 hours fasting blood glucose levels in L-G6PC(−/−) mice 24 hours after injection with 0.1 mg/kg, 0.2 mg/kg, or 0.5 mg/kg of mRNA (SEQ ID NO:5) in lipid nanoparticles. Wild-type mice were injected with Tris-sucrose (WT). Control L-G6PC(−/−) mice were injected with Tris-sucrose (Veh) or mRNA encoding eGFP (0.5 mg/kg). *p<0.05, ****p<0.0001 vs. eGFP; one-way ANOVA followed by Dunnett's multiple comparisons.

FIG. 2B is a bar graph showing 24 hours fasting serum triglyceride levels in L-G6PC(−/−) mice 24 hours after injection with 0.1 mg/kg, 0.2 mg/kg, or 0.5 mg/kg of mRNA (SEQ ID NO:5) in lipid nanoparticles. Wild-type mice were injected with Tris-sucrose (WT). Control L-G6PC(−/−) mice were injected with Tris-sucrose (Veh) or mRNA encoding eGFP (0.5 mg/kg). *p<0.05, ****p<0.0001 vs. eGFP; one-way ANOVA followed by Dunnett's multiple comparisons.

FIG. 3A is a bar graph showing the liver weight of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to liver weight in L-G6PC(−/−) mice injected with Tris-sucrose (Veh) or mRNA encoding eGFP (0.5 mg/kg) or wild-type mice injected with Tris-sucrose (WT). *p<0.05, **p<0.01, ****p<0.0001 vs. eGFP; one-way ANOVA followed by Dunnett's multiple comparisons.

FIG. 3B is a bar graph showing the hepatic G6P content of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to hepatic G6P content in L-G6PC(−/−) mice injected with Tris-sucrose (Veh) or mRNA encoding eGFP (0.5 mg/kg) or wild-type mice injected with Tris-sucrose (WT). *p<0.05, **p<0.01, ****p<0.0001 vs. eGFP; one-way ANOVA followed by Dunnett's multiple comparisons.

FIG. 3C is a bar graph showing the glycogen content of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to glycogen content in L-G6PC(−/−) mice injected with Tris-sucrose (Veh) or mRNA encoding eGFP (0.5 mg/kg) or wild-type mice injected with Tris-sucrose (WT). *p<0.05, **p<0.01, ****p<0.0001 vs. eGFP; one-way ANOVA followed by Dunnett's multiple comparisons.

FIG. 3D is a bar graph showing the hepatic triglyceride level of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to the hepatic triglyceride level in L-G6PC(−/−) mice injected with Tris-sucrose (Veh) or mRNA encoding eGFP (0.5 mg/kg) or wild-type mice injected with Tris-sucrose (WT). *p<0.05, **p<0.01, ****p<0.0001 vs. eGFP; one-way ANOVA followed by Dunnett's multiple comparisons.

FIG. 3E is a bar graph showing the hepatic G6Pase activity of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to the hepatic G6Pase activity in L-G6PC(−/−) mice injected with Tris-sucrose (Veh) or mRNA encoding eGFP (0.5 mg/kg) or wild-type mice injected with Tris-sucrose (WT).

FIG. 3F is a bar graph showing the hepatic human G6Pase protein level of L-G6PC(−/−) mice 24 hours after injection with 0.1 mg/kg, 0.2 mg/kg, or 0.5 mg/kg of mRNA (SEQ ID NO:5) in lipid nanoparticles. Results were compared to the hepatic human G6Pase protein level in L-G6PC(−/−) mice injected with Tris-sucrose (Veh) or mRNA encoding eGFP (0.5 mg/kg) or wild-type mice injected with Tris-sucrose (WT).

FIG. 4 is a graph showing fasting blood glucose levels in L-G6PC(−/−) mice injected at 14 day intervals with a total of three doses of 0.2 mg/kg of mRNA (SEQ ID NO:5) in lipid nanoparticles. Wild-type mice were injected with Tris-sucrose (WT, Veh). Control L-G6PC(−/−) mice were injected with Tris-sucrose (Veh) or mRNA encoding eGFP (0.2 mg/kg).

FIG. 5A is a bar graph showing 2.5 hours fasting blood glucose levels in L-G6PC(−/−) mice 24 hours after injection with the first of three doses of 0.2 mg/kg of mRNA (SEQ ID NO:5) in lipid nanoparticles. Wild-type mice were injected with Tris-sucrose. Control L-G6PC(−/−) mice were injected with Tris-sucrose or mRNA encoding eGFP (0.2 mg/kg). ***p<0.001 vs. eGFP group.

FIG. 5B is a bar graph showing 2.5 hours fasting blood glucose levels in L-G6PC(−/−) mice 24 hours after injection with the third of three doses of 0.2 mg/kg of mRNA (SEQ ID NO:5) in lipid nanoparticles. Wild-type mice were injected with Tris-sucrose. Control L-G6PC(−/−) mice were injected with Tris-sucrose or mRNA encoding eGFP (0.2 mg/kg). ***p<0.001 vs. eGFP group.

FIG. 6A is a bar graph showing 24 hours fasting blood glucose levels in L-G6PC(−/−) mice 24 hours after injection with the first of three doses of 0.2 mg/kg of mRNA (SEQ ID NO:5) in lipid nanoparticles. Wild-type mice were injected with Tris-sucrose. Control L-G6PC(−/−) mice were injected with Tris-sucrose or mRNA encoding eGFP (0.2 mg/kg). **p<0.01, ****p<0.0001 vs. eGFP group.

FIG. 6B is a bar graph showing 24 hours fasting blood glucose levels in L-G6PC(−/−) mice 24 hours after injection with the third of three doses of 0.2 mg/kg of mRNA (SEQ ID NO:5) in lipid nanoparticles. Wild-type mice were injected with Tris-sucrose. Control L-G6PC(−/−) mice were injected with Tris-sucrose or mRNA encoding eGFP (0.2 mg/kg). **p<0.01, ****p<0.0001 vs. eGFP group.

FIG. 7A is a bar graph showing the number of mice with tumors and tumor size in wild-type (WT) or L. G6PC−/− mice. The mice were injected with ten doses (0.25 mg/kg; administered every two weeks) of hG6PC S298C mRNA (SEQ ID NO:6) or control eGFP mRNA in lipid nanoparticles. Results are expressed as mean±SD (n=5-14).

FIG. 7B is a series of bar graphs showing protein expression levels of HCA/HCC related biomarkers PKM2, β-catenin, and p62 in the livers of the mice of FIG. 7A. Results are expressed as mean±SD (n=5-14). For statistical analysis, raw values were log 2 transformed and subjected to one-way ANOVA, followed by the Dunnett's multiple comparisons test, compared to the eGFP mRNA treated group (* p≤0.05, ** p≤0.01, *** p≤0.001, **** p≤0.0001).

FIG. 7C is a series of bar graphs showing mRNA expression levels of genes related to HCA/HCC biomarkers (Gpc3, Tgfb1, GluI, and Ctnnb1) in the livers of the mice of FIG. 7A. Results are expressed as mean±SD (n=5-14). For statistical analysis, raw values were log 2 transformed and subjected to one-way ANOVA, followed by the Dunnett's multiple comparisons test, compared to the eGFP mRNA treated group (* p≤0.05, ** p<0.01, *** p≤0.001, **** p≤0.0001).

FIG. 7D is a bar graph showing serum biomarker for GSD1a (blood glucose levels) and HCA/HCC (AFP and CRP levels) of the mice of FIG. 7A. Results are expressed as mean±SD (n=5-14). For statistical analysis, raw values were log 2 transformed and subjected to one-way ANOVA, followed by the Dunnett's multiple comparisons test, compared to the eGFP mRNA treated group (* p<0.05, ** p<0.01, *** p≤0.001, **** p≤0.0001).

FIG. 8 is the complete sequence of the mRNA encoding human G6PC-S298C (SEQ ID NO:6) used in FIGS. 7A-7D. The 5′ and 3′ UTRs are underlined.

### DETAILED DESCRIPTION

The present disclosure provides lipid nanoparticle/mRNA therapeutics for the treatment of GSD-Ia. Lipid nanoparticles are an ideal platform for the safe and effective delivery of mRNAs to target cells. Lipid nanoparticles have the unique ability to deliver nucleic acids by a mechanism involving cellular uptake, intracellular transport and endosomal release or endosomal escape.

### Glucose-6-Phosphatase (G6PC)

Glucose-6-phosphatase (G6Pase or G6PC, EC 3.1.3.9) catalyzes the hydrolysis of glucose-6-phosphate (G6P) to glucose and phosphate (Pi) in the terminal step of gluconeogenesis and glycogenolysis. G6PC is primarily present in the liver, and to a lesser extent in the kidneys and intestine. G6PC, encoded by the G6PC gene, is a multi-subunit integral membrane protein that resides on the endoplasmic reticulum (ER) membrane, and it hydrolyzes G6P to glucose and Pi in the ER lumen. The G6PC protein is 357 amino acids in length, with a molecular mass of about 40.5 kDa. It is composed of a catalytic subunit, and transporters for G6P, inorganic phosphate (Pi) and glucose. The amino acid sequence of human G6PC with a S298C substitution is provided in SEQ ID NO:1. The disclosure provides an mRNA comprising an open reading frame encoding human G6PC S298C (SEQ ID NO:1).

mRNAs, Open Reading Frames, and Untranslated Regions

The instant invention features mRNAs for use in treating or preventing a GSD-I such as GSD-Ia. The mRNAs featured for use in the invention are administered to subjects and encode human G6PC S298C protein (SEQ ID NO:1) in vivo. The invention relates to an mRNA comprising an open reading frame (ORF) of linked nucleosides encoding human G6PC S298C (SEQ ID NO:1). In some embodiments, the ORF is at least 80%, 85%, 90%, 95%, 98%, or 99% identical to SEQ ID NO:2. In some embodiments, the ORF is identical to SEQ ID NO:2.

In some embodiments, the mRNA comprises a sequence-optimized ORF encoding human G6PC S298C protein (SEQ ID NO:1), wherein the mRNA comprises at least one chemically modified nucleobase, e.g., N1-methylpseudouracil or 5-methoxyuracil. In certain embodiments, all uracils in the mRNA are N1-methylpseudouracils. In other embodiments, all uracils in the mRNA are 5-methoxyuracils. In some embodiments, the mRNA further comprises a miRNA binding site, e.g., a miRNA binding site that binds to miR-142 and/or a miRNA binding site that binds to miR-126.

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854).

### Untranslated Regions (UTRs)

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the invention comprising an open reading frame (ORF) encoding a G6PC polypeptide further comprises UTR (e.g., a 5′ UTR or functional fragment thereof, a 3′ UTR or functional fragment thereof, or a combination thereof).

A UTR (e.g., 5′ UTR or 3′ UTR) can be homologous or heterologous to the coding region in a polynucleotide. In some embodiments, the UTR is homologous to the ORF encoding the G6PC polypeptide. In some embodiments, the UTR is heterologous to the ORF encoding the G6PC polypeptide.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 214), where R is a purine (adenine or guanine) three bases upstream of the start codon (AUG), which is followed by another ‘U. 5’ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein AB/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (1713) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

a. 5′ UTR Sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide, e.g., mRNA, comprising an open reading frame encoding a G6PC polypeptide (e.g., SEQ ID NO:1), which polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table 1 or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table 1 or a variant or fragment thereof (e.g., a functional variant or fragment thereof).

In an embodiment, the polynucleotide having a 5′ UTR sequence provided in Table 1 or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19 or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table 1 or a variant or fragment thereof, results in an increased level and/or activity, e.g., output, of the polypeptide encoded by the polynucleotide. In an embodiment, the 5′UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19 or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table 1 or a variant or fragment thereof.

In an embodiment, the increase in half-life of the polynucleotide is measured according to an assay that measures the half-life of a polynucleotide.

In an embodiment, the increase in level and/or activity, e.g., output, of the polypeptide encoded by the polynucleotide is measured according to an assay that measures the level and/or activity of a polypeptide.

In an embodiment, the 5′ UTR comprises a sequence provided in Table 1 or a sequence with at least 80%, 85%, 90%, 95%, 96%, 97%, 98%, 99% or 100% identity to a 5′ UTR sequence provided in Table 1, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In an embodiment, the 5′ UTR comprises a sequence with at least 80%, 85%, 90%, 95%, 96%, 97%, 98%, 99% or 100% identity to SEQ ID NO: 50. In an embodiment, the 5′ UTR comprises a sequence with at least 80%, 85%, 90%, 95%, 96%, 97%, 98%, 99% or 100% identity to SEQ ID NO: 51. In an embodiment, the 5′ UTR comprises a sequence with at least 80%, 85%, 90%, 95%, 96%, 97%, 98%, 99% or 100% identity to SEQ ID NO: 52. In an embodiment, the 5′ UTR comprises a sequence with at least 80%, 85%, 90%, 95%, 96%, 97%, 98%, 99% or 100% identity to SEQ ID NO: 53. In an embodiment, the 5′ UTR comprises a sequence with at least 80%, 85%, 90%, 95%, 96%, 97%, 98%, 99% or 100% identity to SEQ ID NO: 54. In an embodiment, the 5′ UTR comprises a sequence with at least 80%, 85%, 90%, 95%, 96%, 97%, 98%, 99% or 100% identity to SEQ ID NO: 55. In an embodiment, the 5′ UTR comprises a sequence with at least 80%, 85%, 90%, 95%, 96%, 97%, 98%, 99% or 100% identity to SEQ ID NO: 56. In an embodiment, the 5′ UTR comprises a sequence with at least 80%, 85%, 90%, 95%, 96%, 97%, 98%, 99% or 100% identity to SEQ ID NO: 57. In an embodiment, the 5′ UTR comprises a sequence with at least 80%, 85%, 90%, 95%, 96%, 97%, 98%, 99% or 100% identity to SEQ ID NO: 58.

In an embodiment, the 5′ UTR comprises the sequence of SEQ ID NO:58. In an embodiment, the 5′ UTR consists of the sequence of SEQ ID NO:58.

In an embodiment, a 5′ UTR sequence provided in Table 1 has a first nucleotide which is an A. In an embodiment, a 5′ UTR sequence provided in Table 1 has a first nucleotide which is a G.

TABLE 1
5′ UTR sequences

| | Sequence | |
|---|---|---|
| SEQ ID NO: | name | Sequence |
| 50 | A1 | GGAAAUCGCAAAAUUUGCUCUUCGCGUUAGAUUUCUUUUA |
| | | GUUUUCUCGCAACUAGCAAGCUUUUUGUUCUCGCC |
| 51 | A5 | GGAAAUCCCCACAACCGCCUCAUAUCCAGGCUCAAGAAUA |
| | | GAGCUCAGUGUUUUGUUGUUUAAUCAUUCCGACGUGUUUU |
| | | GCGAUAUUCGCGCAAAGCAGCCAGUCGCGCGCUUGCUUUU |
| | | AAGUAGAGUUGUUUUUCCACCCGUUUGCCAGGCAUCUUUA |
| | | AUUUAACAUAUUUUUAUUUUUCAGGCUAACCUACGCCGCC |
| | | ACC |
| 52 | A6 | GGAAAUAAGAGAGAAAAGAAGAGUAAGAAGAAAUAUAAGA |
| | | UCUCCCUGAGCUUCAGGGAGCCCCGGCGCCGCCACC |
| 53 | A7 | GGAAACCCCCCACCCCCGUAAGAGAGAAAAGAAGAGUAAG |
| | | AAGAAAUAUAAGAUCUCCCUGAGCUUCAGGGAGCCCCGGC |
| | | GCCGCCACC |
| 54 | A8 | GGAGAACUUCCGCUUCCGUUGGCGCAAGCGCUUUCAUUUU |
| | | UUCUGCUACCGUGACUAAG |
| 55 | A9 | GGAAAUAAGAGAGAAAAGAAGAGUAAGAAGAAAUALAAGA |
| | | GCCACC |
| 56 | A11 | GGAAAUAAGAGAGAAAAGAAGAGUAAGAAGAAAUAUAAGA |
| | (Reference) | CCCCGGCGCCGCCACC |
| 57 | A2 | GGAAAUCGCAAAAUUUGCUCUUCGCGUUAGAUUUCUUUUA |
| | | GUUUUCUCGCAACUAGCAAGCUUUUUGUUCUCGCCGCCGC |
| | | C |
| 58 | A3 | GGAAAUCGCAAAAUUUUCUUUUCGCGUUAGAUUUCUUUUA |
| | | GUUUUCUUUCAACUAGCAAGCUUUUUGUUCUCGCCGCCGC |
| | | C |
| 59 | A4 | G G A A A U C G C A A A A (N₂)ₓ (N₃)ₓ C U |
| | | (N₄)ₓ (N₅)ₓ C G C G U U A G A U U U C U U |
| | | U U A G U U U U C U N₆ N₇ C A A C U A G C |
| | | A A G C U U U U U G U U C U C G C C (N₈ |
| | | C C)x |
| | | (N₂)ₓ is a uracil and x is an integer from |
| | | 0 to 5, e.g., wherein x = 3 or 4; |
| | | (N₃)ₓ is a guanine and x is an integer |
| | | from 0 to 1; |
| | | (N₄)ₓ is a cytosine and x is an integer |
| | | from 0 to 1; |
| | | (N₅)ₓ is a uracil and x is an integer |
| | | from 0 to 5, e.g., wherein x = 2 or 3; |
| | | N₆ is a uracil or cytosine; |
| | | N₇ is a uracil or guanine; |
| | | N₈ is adenine or guanine and x is an |
| | | integer from 0 to 1. |
| 60 | A27 | GGAAAAUUUUAGCCUGGAACGUUAGAUAACUGUCCUGUUG |
| 61 | A12 | GGAAACUUUAUUUAGUGUUACUUUAUUUUCUGUUUAUUUG |
| | | UGUUUCUUCAGUGGGUUUGUUCUAAUUUCCUUGGCCGCC |
| 62 | A13 | GGAAAAUCUGUAUUAGGUUGGCGUGUUCUUUGGUCGGUUG |
| | | UUAGUAUUGUUGUUGAUUCGUUUGUGGUCGGUUGCCGCC |
| 63 | A14 | GGAAAAUUAUUAACAUCUUGGUAUUCUCGAUAACCAUUCG |
| | | UUGGAUUUUAUUGUAUUCGUAGUUUGGGUUCCUGCCGCC |
| 64 | A15 | GGAAAUUAUUAUUAUUUCUAGCUACAAUUUAUCAUUGUAU |
| | | UAUUUUAGCUAUUCAUCAUUAUUUACUUGGUGAUCAACA |
| 65 | A16 | GGAAAUAGGUUGUUAACCAAGUUCAAGCCUAAUAAGCUUG |
| | | GAUUCUGGUGACUUGCUUCACCGUUGGCGGGCACCGAUC |
| 66 | A17 | GGAAAUCGUAGAGAGUCGUACUUAGUACAUAUCGACUAUC |
| | | GGUGGACACCAUCAAGAUUAUAAACCAGGCCAGA |
| 67 | A18 | GGAAACCCGCCCAAGCGACCCCAACAUAUCAGCAGUUGCC |
| | | CAAUCCCAACUCCCAACACAAUTCCCCAAGCAACGCCGCC |
| 68 | A19 | GGAAAGCGAUUGAAGGCGUCUUUUCAACUACUCGAUUAAG |
| | | GUUGGGUAUCGUCGUGGGACUUGGAAAUUUGUUGUUUCC |
| 69 | A20 | GGAAACUAAUCGAAAUAAAAGAGCCCCGUACUCUUUUAUU |
| | | UCUAUUAGGUUAGGAGCCUUAGCAUUUGUAUCUUAGGUA |
| 70 | A21 | GGAAAUGUGAUUUCCAGCAACUUCUUUUGAAUAUAUUGAA |
| | | UUCCUAAUUCAAAGCGAACAAAUCUACAAGCCAUAUACC |
| 71 | A22 | GGAAAUCGUAGAGAGUCGUACUUACGUGGUCGCCAUUGCA |
| | | UAGCGCGCGAAAGCAACAGGAACAAGAACGCGCC |
| 72 | A23 | GGAAAUCGUAGAGAGUCGUACUUAGAAUAAACAGAGUCGG |
| | | GUCGACUUGUCUCUGAUACUACGACGUCACAAUC |
| 73 | A24 | GGAAAAUUUGCCUUCGGAGUUGCGUAUCCUGAACUGCCCA |
| | | GCCUCCUGAUAUACAACUGUUCCGCUUAUUCGGGCCGCC |
| 74 | A25 | GGAAAUCUGAGCAGGAAUCCUUUGUGCAUUGAAGACUUUA |
| | | GAUUCCUCUCUGCGGUAGACGUGCACUUAUAAGUAUUUG |
| 75 | A26 | GGAAAGCGAUUGAAGGCGUCUUUUCAACUACUCGAUUAAG |
| | | GUUGGGUAUCGUCGUGGGACUUGGAAAUUUGUUGCCACC |
| 76 | A28 | GGAAAUUUUUUUUUGAUAUUAUAAGAGUUUUUUUUUGAUA |
| | | UUAAGAAAAUUUUUUUUUGAUAUUAGAAGAGUAAGAAGAA |
| | | AUAUAAGACCCCGGCGCCGCCACC |
| 77 | A29 | GGAAAUAAGAGAGAAAAGAAGAGUAAGAAGAAAUAUAAGA |
| | | GCCAAAAAAAAAAAACC |
| 78 | A30 | GGAAAUCUCCCUGAGCUUCAGGGAGUAAGAGAGAAAAGAA |
| | | GAGUAAGAAGAAAUAUAAGACCCCGGCGCCGCCACC |
| 79 | A31 | GCCRCC, wherein R = A or G |

In an embodiment, the 5′ UTR comprises a variant of SEQ ID NO: 50. In an embodiment, the variant of SEQ ID NO: 50 comprises a nucleic acid sequence of Formula A:

(SEQ ID NO: 59)
G G A A A U C G C A A A A (N₂)x(N₃)x C U(N₄)x(N₅)x
C G C G U U A G A U U U C U U U U A G U U U U C U
N₆ N₇ C A A C U A G C A A G C U U U U U G U U C U
C G C C (N₈ C C)x,

wherein:

(N2)ₓ is a uracil and x is an integer from 0 to 5, e.g., wherein x=3 or 4;

(N3)ₓ is a guanine and x is an integer from 0 to 1;

(N4)ₓ is a cytosine and x is an integer from 0 to 1;

(N5)ₓ is a uracil and x is an integer from 0 to 5, e.g., wherein x=2 or 3;

N6 is a uracil or cytosine;

N7 is a uracil or guanine;

N8 is adenine or guanine and x is an integer from 0 to 1.

In an embodiment (N2)ₓ is a uracil and x is 0. In an embodiment (N2)ₓ is a uracil and x is 1. In an embodiment (N2)ₓ is a uracil and x is 2. In an embodiment (N2)ₓ is a uracil and x is 3. In an embodiment, (N2)ₓ is a uracil and x is 4. In an embodiment (N2)ₓ is a uracil and x is 5.

In an embodiment, (N3)ₓ is a guanine and x is 0. In an embodiment, (N3)ₓ is a guanine and x is 1.

In an embodiment, (N4)ₓ is a cytosine and x is 0. In an embodiment, (N4)ₓ is a cytosine and x is 1.

In an embodiment (N5)ₓ is a uracil and x is 0. In an embodiment (N5)ₓ is a uracil and x is 1. In an embodiment (N5)ₓ is a uracil and x is 2. In an embodiment (N5)ₓ is a uracil and x is 3. In an embodiment, (N5)ₓ is a uracil and x is 4. In an embodiment (N5)ₓ is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO: 50. In an embodiment, the variant of SEQ ID NO: 50 comprises a sequence with at least 50%, 60%, 70%, 80%, 85%, 90%, 95%, 96%, 97%, 98%, or 99% identity to SEQ ID NO: 50. In an embodiment, the variant of SEQ ID NO: 50 comprises a sequence with at least 50% identity to SEQ ID NO: 50. In an embodiment, the variant of SEQ ID NO: 50 comprises a sequence with at least 60% identity to SEQ ID NO: 50. In an embodiment, the variant of SEQ ID NO: 50 comprises a sequence with at least 70% identity to SEQ ID NO: 50. In an embodiment, the variant of SEQ ID NO: 50 comprises a sequence with at least 80% identity to SEQ ID NO: 50. In an embodiment, the variant of SEQ ID NO: 50 comprises a sequence with at least 90% identity to SEQ ID NO: 50. In an embodiment, the variant of SEQ ID NO: 50 comprises a sequence with at least 95% identity to SEQ ID NO: 50. In an embodiment, the variant of SEQ ID NO: 50 comprises a sequence with at least 96% identity to SEQ ID NO: 50. In an embodiment, the variant of SEQ ID NO: 50 comprises a sequence with at least 97% identity to SEQ ID NO: 50. In an embodiment, the variant of SEQ ID NO: 50 comprises a sequence with at least 98% identity to SEQ ID NO: 50. In an embodiment, the variant of SEQ ID NO: 50 comprises a sequence with at least 99% identity to SEQ ID NO: 50.

In an embodiment, the variant of SEQ ID NO: 50 comprises a uridine content of at least 5%, 10%, 20%, 30%, 40%, 50%, 60%, 70%, or 80%. In an embodiment, the variant of SEQ ID NO: 50 comprises a uridine content of at least 5%. In an embodiment, the variant of SEQ ID NO: 50 comprises a uridine content of at least 10%. In an embodiment, the variant of SEQ ID NO: 50 comprises a uridine content of at least 20%. In an embodiment, the variant of SEQ ID NO: 50 comprises a uridine content of at least 30%. In an embodiment, the variant of SEQ ID NO: 50 comprises a uridine content of at least 40%. In an embodiment, the variant of SEQ ID NO: 50 comprises a uridine content of at least 50%. In an embodiment, the variant of SEQ ID NO: 50 comprises a uridine content of at least 60%. In an embodiment, the variant of SEQ ID NO: 50 comprises a uridine content of at least 70%. In an embodiment, the variant of SEQ ID NO: 50 comprises a uridine content of at least 80%.

In an embodiment, the variant of SEQ ID NO: 50 comprises at least 2, 3, 4, 5, 6 or 7 consecutive uridines (e.g., a polyuridine tract). In an embodiment, the polyuridine tract in the variant of SEQ ID NO: 50 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO: 50 comprises 4 consecutive uridines. In an embodiment, the polyuridine tract in the variant of SEQ ID NO: 50 comprises 5 consecutive uridines.

In an embodiment, the variant of SEQ ID NO: 50 comprises 1, 2, 3, 4, 5, 6, 7, 8, 9, 10, 11, 12, 13, 14, or 15 polyuridine tracts. In an embodiment, the variant of SEQ ID NO: 50 comprises 3 polyuridine tracts. In an embodiment, the variant of SEQ ID NO: 50 comprises 4 polyuridine tracts. In an embodiment, the variant of SEQ ID NO: 50 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.

In an embodiment, a first polyuridine tract and a second polyuridine tract are adjacent to each other.

In an embodiment, a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence (SEQ ID NO: 79) wherein R is an adenine or guanine. In an embodiment, the Kozak sequence is disposed at the 3′ end of the 5′UTR sequence.

In an aspect, the polynucleotide (e.g., mRNA) comprising an open reading frame encoding a G6PC polypeptide (e.g., SEQ ID NO:1) and comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating G6PC-related disease, disorder, or condition, e.g., GSD-Ia in a subject.

In an aspect, an LNP composition comprising a polynucleotide disclosed herein encoding a G6PC polypeptide, e.g., as described herein, can be administered with an additional agent, e.g., as described herein.

b. 3′ UTR Sequences

3′UTR sequences have been shown to influence translation, half-life, and subcellular localization of mRNAs (Mayr C., Cold Spring Harb Persp Biot 2019 Oct. 1; 11(10):a034728).

Disclosed herein, inter alia, is a polynucleotide, e.g., mRNA, comprising an open reading frame encoding a G6PC polypeptide (e.g., SEQ ID NO:1), which polynucleotide has a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as described herein); (b) a coding region comprising a stop element (e.g., as described herein); and (c) a 3′-UTR (e.g., as provided in Table 2 or a variant or fragment thereof), and LNP compositions comprising the same. In an embodiment, the polynucleotide comprises a 3′-UTR comprising a sequence provided in Table 2 or a variant or fragment thereof.

In an embodiment, the polynucleotide having a 3′ UTR sequence provided in Table 2 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in Table 2 or a variant or fragment thereof, results in a polynucleotide with a mean half-life score of greater than 10.

In an embodiment, the polynucleotide having a 3′ UTR sequence provided in Table 2 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of Table 2 or a variant or fragment thereof.

In an embodiment, the polynucleotide comprises a 3′ UTR sequence provided in Table 2 or a sequence with at least 80%, 85%, 90%, 95%, 96%, 97%, 98%, 99% or 100% identity to a 3′ UTR sequence provided in Table 2, or a fragment thereof. In an embodiment, the 3′ UTR comprises a sequence with at least 80%, 85%, 90%, 95%, 96%, 97%, 98%, 99% or 100% identity to SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, or SEQ ID NO:115.

In an embodiment, the 3′ UTR comprises the sequence of SEQ ID NO: 100, or a sequence with at least 80%, 85%, 90%, 95%, 96%, 97%, 98%, 99% or 100% identity to SEQ ID NO: 100. In an embodiment, the 3′ UTR comprises the sequence of SEQ ID NO: 101, or a sequence with at least 80%, 85%, 90%, 95%, 96%, 97%, 98%, 99% or 100% identity to SEQ ID NO: 101. In an embodiment, the 3′ UTR comprises the sequence of SEQ ID NO: 102, or a sequence with at least 80%, 85%, 90%, 95%, 96%, 97%, 98%, 99% or 100% identity to SEQ ID NO: 102. In an embodiment, the 3′ UTR comprises the sequence of SEQ ID NO: 103, or a sequence with at least 80%, 85%, 90%, 95%, 96%, 97%, 98%, 99% or 100% identity to SEQ ID NO: 103. In an embodiment, the 3′ UTR comprises the sequence of SEQ ID NO: 104, or a sequence with at least 80%, 85%, 90%, 95%, 96%, 97%, 98%, 99% or 100% identity to SEQ ID NO: 104. In an embodiment, the 3′ UTR comprises the sequence of SEQ ID NO: 105, or a sequence with at least 80%, 85%, 90%, 95%, 96%, 97%, 98%, 99% or 100% identity to SEQ ID NO: 105. In an embodiment, the 3′ UTR comprises the sequence of SEQ ID NO: 106, or a sequence with at least 80%, 85%, 90%, 95%, 96%, 97%, 98%, 99% or 100% identity to SEQ ID NO: 106. In an embodiment, the 3′ UTR comprises the sequence of SEQ ID NO: 107, or a sequence with at least 80%, 85%, 90%, 95%, 96%, 97%, 98%, 99% or 100% identity to SEQ ID NO: 107. In an embodiment, the 3′ UTR comprises the sequence of SEQ ID NO: 108, or a sequence with at least 80%, 85%, 90%, 95%, 96%, 97%, 98%, 99% or 100% identity to SEQ ID NO: 108. In an embodiment, the 3′ UTR comprises the sequence of SEQ ID NO: 109, or a sequence with at least 80%, 85%, 90%, 95%, 96%, 97%, 98%, 99% or 100% identity to SEQ ID NO: 109. In an embodiment, the 3′ UTR comprises the sequence of SEQ ID NO: 110, or a sequence with at least 80%, 85%, 90%, 95%, 96%, 97%, 98%, 99% or 100% identity to SEQ ID NO: 110. In an embodiment, the 3′ UTR comprises the sequence of SEQ ID NO: 111, or a sequence with at least 80%, 85%, 90%, 95%, 96%, 97%, 98%, 99% or 100% identity to SEQ ID NO: 111. In an embodiment, the 3′ UTR comprises the sequence of SEQ ID NO: 112, or a sequence with at least 80%, 85%, 90%, 95%, 96%, 97%, 98%, 99% or 100% identity to SEQ ID NO: 112. In an embodiment, the 3′ UTR comprises the sequence of SEQ ID NO: 113, or a sequence with at least 80%, 85%, 90%, 95%, 96%, 97%, 98%, 99% or 100% identity to SEQ ID NO: 113. In an embodiment, the 3′ UTR comprises the sequence of SEQ ID NO: 114, or a sequence with at least 80%, 85%, 90%, 95%, 96%, 97%, 98%, 99% or 100% identity to SEQ ID NO: 114. In an embodiment, the 3′ UTR comprises the sequence of SEQ ID NO: 115, or a sequence with at least 80%, 85%, 90%, 95%, 96%, 97%, 98%, 99% or 100% identity to SEQ ID NO: 115.

TABLE 2
3′ UTR sequences

| | Sequence | |
|---|---|---|
| SEQ ID NO | information | Sequence |
| 100 | B1 | UAAUAGUAAGCUGGAGCCUCCUGAGAGACCUGUGUGAACUAUU |
| | | GAGAAGAUCGGAACAGCUCCUUACUCUGAGGAAGUUGGUACCC |
| | | CCGUGGUCUUUGAAUAAAGUCUGAGUGGGCGGC |
| 101 | B3 | UAAUAGUAAGCUGGAGCCUCACUCUCCUCUCCAUCCCGUAUCC |
| | | AGGCUGUGAAUUUUUCAAGGAAUAUAAAGAUCGGGAUGUACCC |
| | | CCGUGGUCUUUGAAUAAAGUCUGAGUGGGCGGC |
| 102 | B4 | UGAUAGUAAGCUGGAGCCUCUAGUGACGGCAACAGGGCUUGGU |
| | | UUUUCCUUGUUGUGAAAUCGACAUCUCUGAAGACAGGGUACCC |
| | | CCGUGGUCUUUGAAUAAAGUCUGAGUGGGCGGC |
| 103 | B5 | UGAUAGUAAGCUGGAGCCUCCUUCCAUCUAGUCACAAAGACUC |
| | | CUUCGUCCCCAGUUGCCGUCUAGGAUUGGGCCUCCCAGUACCC |
| | | CCGUGGUCUUUGAAUAAAGUCUGAGUGGGCGGC |
| 104 | B6 | UGAUAGUAAGCUGGAGCCUCCCAUAACAUGACAUAUCUGGAUU |
| | | UUGUGCUUAGAACCUUAAAUUGGAAGCAUUCUUAAUUGUACCC |
| | | CCGUGGUCUUUGAAUAAAGUCUGAGUGGGCGGC |
| 105 | B7 | UAAUAGUAAGCUGGAGCCUCCGGAAAACUAAAAUAGAGAUAUU |
| | | UCAAGAUUUUAUAAUUUUCAAAGACCUUUGAAAUAUUGUACCC |
| | | CCGUGGUCUUUGAAUAAAGUCUGAGUGGGCGGC |
| 106 | B8 | UAAUAGUAAGCUGGAGCCUCUACACAUUGCUUCUAGUUGGCAG |
| | | AAAUAAUUGAUUAAAAGACCAGAAACUGUGAUAACUGGUACCC |
| | | CCGUGGUCUUUAAAUAAAGUCUAAGUGGGCGGC |
| 107 | B9 | UGAUAAUAGGCUGGAGCCUCGGUGGCCAUGCUUCUUGCCCCUU |
| | | GGGCCUCCCCCCAGCCCCUCCUCCCCUUCCUGCACCCGUACCC |
| | | CCGUGGUCUUUGAAUAAAGUCUGAGUGGGCGGC |
| 108 | B10 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | CCGUGGUCUUUGAAUAAAGUCUGAGUGGGCGGC |
| 109 | B11 | UGAUAAUAGGCUGGAGCCUCGGUGGCCAUGCUUCUUGCCCCUU |
| | | GGGCCUCCCCCCAGCCCCUCCUCCCCUUCCUGCACCCGUACCC |
| | | CCCAAACACCAUUGUCACACUCCAGUGGUCUUUGAAUAAAGUC |
| | | UGAGUGGGCGGC |
| 110 | B12 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | CCCAAACACCAUUGUCACACUCCAGUGGUCUUUGAAUAAAGUC |
| | | UGAGUGGGCGGC |
| 111 | B13 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | CCUCCAUAAAGUAGGAAACACUACAGUGGUCUUUGAAUAAAGU |
| | | CUGAGUGGGCGGC |
| 112 | B14 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | CCCGCAUUAUUACUCACGGUACGAGUGGUCUUUGAAUAAAGUC |
| | | UGAGUGGGCGGC |
| 113 | B15 | UGAUAAUAGUCCAUAAAGUAGGAAACACUACAGCUGGAGCCUC |
| | | GGUGGCCAUGCUUCUUGCCCCUUGGGCCUCCCCCCAGCCCCUC |
| | | CUCCCCUUCCUGCACCCGUACCCCCCGCAUUAUUACUCACGGU |
| | | ACGAGUGGUCUUUGAAUAAAGUCUGAGUGGGCGGC |
| 114 | B16 | UGAUAAUAGUCCAUAAAGUAGGAAACACUACAGCUGGAGCCUC |
| | | GGUGGCCUAGCUUCUUGCCCCUUGGGCCUCCAUAAAGUAGGAA |
| | | ACACUACAUCCCCCCAGCCCCUCCUCCCCUUCCUGCACCCGUA |
| | | CCCCCUCCAUAAAGUAGGAAACACUACAGUGGUCUUUGAAUAA |
| | | AGUCUGAGUGGGCGGC |
| 115 | B2 | CUGAGAGACCUGUGUGAACUAUUGAGAAGAUCGGAACAGCUCC |
| | | UUACUCUGAGGAAGUUG |
| 116 | B17 | UGAUAAUAGGCUGGAGCCUCUCACACACCUCUGCCCCUUGGGC |
| | | CUCCCACUCCCAUGGCUCUGGGCGGUCCAGAAGGAGCGUACCC |
| | | CCGUGGUCUUUGAAUAAAGUCUGAGUGGGCGGC |
| 117 | B18 | UGAUAAUAGGCUGGAGCCUCCACCGCGUUAUCCGUUCCUCGUA |
| | | GGCUGGUCCUGGGGAACGGGUCGGCGGGUACCCCCGUGGUCUU |
| | | UGAAUAAAGUCUGAGUGGGCGGC |
| 118 | B19 | UGAUAAUAGGCUGGAGCCUCUGCCCGGCAACGGCCAGGUCUGU |
| | | GCCAAGUGUUUGCUGACGCAACCCCCACUGGCUGGGGCUUGGU |
| | | CAUGGGCCAUCAGCGCGUGCGUGGAACCUUUUCGGCUCCUCUG |
| | | CCGAUCCAUACUGCGGAACUCCUAGCCGCUUGUUUUGCUCGCA |
| | | GCAGGUCUGGAGCAAACAUUAUCGGGACUGAUAACUCUGUUGU |
| | | CCUGUACCCCCGUGGUCUUUGAAUAAAGUCUGAGUGGGCGGC |
| 119 | B20 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | UUUUUUUUUUUUUUUUUUUCUUCUUUUCUUUUUUUUCUUUUUU |
| | | UUUUUUCUUUCUUUUUUUCUUUUUUUUUCUUUUCUUUUUUCUU |
| | | UUUUUUUUUUUUUUCCGUGGUCUUUGAAUAAAGUCUGAGUGGG |
| | | CGGC |
| 120 | B21 | UGAUAAUAGGCUGGAGCCUCGGUGGCCUAGCUUCUUGCCCCUU |
| | | GGGCCUCCCCCCAGCCCCUCCUCCCCUUCCUGCACCCGUACCC |
| | | UUUUUUUUUUUUUUUUUUUUUUUUUUUUUUUUUUUUUUUUUUU |
| | | UUUUUUUUUUUUUUUUUUUUUUUUUUUUUUUUUUUUUUUUUUU |
| | | UUUUUUUUUUUUUUCCGUGGUCUUUGAAUAAAGUCUGAGUGGG |
| | | CGGC |
| 133 | B22 | UAAGUCUCCAUAAAGUAGGAAACACUACAGCUGGAGCCUCGGU |
| | | GGCCUAGCUUCUUGCCCCUUGGGCCUCCAUAAAGUAGGAAACA |
| | | CUACAUCCCCCCAGCCCCUCCUCCCCUUCCUGCACCCGUACCC |
| | | CCUCCAUAAAGUAGGAAACACUACAGUGGUCUUUGAAUAAAGU |
| | | CUGAGUGGGCGGC |
| 134 | B23 | UAAAGCGCUGGAGCCUCGGUGGCCUAGCUUCUUGCCCCUUGGG |
| | | CCUCCCCCCAGCCCCUCCUCCCCUUCCUGCACCCGUACCCCCU |
| | | CCAUAAAGUAGGAAACACUACAGUGGUCUUUGAAUAAAGUCUG |
| | | AGUGGGCGGC |
| 135 | B24 | UAAAGCUCCCCGGGGUCCAUAAAGUAGGAAACACUACAGCUGG |
| | | AGCCUCCUGAGAGACCUGUGUGAACUAUUGAGAAGAUCGGAAC |
| | | AGCUCCUUACUCUGAGGAAGUUGUCCAUAAAGUAGGAAACACU |
| | | ACAGUACCCCCUCCAUAAAGUAGGAAACACUACAGUGGUCUUU |
| | | GAAUAAAGUCUGAGUGGGCGGC |
| 136 | B25 | UAAUAGUAAACCUCACUCACGGCCACAUUGAGUGCCAGGCUCC |
| | | GGGCUGGUUUAUAGUAGUGUAGAGCAUUGCAGCACUUAGACUG |
| | | GGGUGCUGUAGUCUUUAUUGUAGUCUUUCCACAUACCUGAUAA |
| | | UUCUUAGAUAAUUUCUUAUUUUAAUUCCAUAAAGUAGGAAACA |
| | | CUACAUAAAUCUCCAUAAAGUAGGAAACACUACAUAUUCUUCC |
| | | AUAAAGUAGGAAACACUACAUAGGCU |

In an embodiment, the 3′ UTR comprises a micro RNA (miRNA) binding site, e.g., as described herein, which binds to a miR present in a human cell. In an embodiment, the 3′ UTR comprises a miRNA binding site of SEQ ID NO: 212, SEQ ID NO: 174, SEQ ID NO: 152 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

(SEQ ID NO: 212)
miR122 bs = CAAACACCAUUGUCACACUCCA
(SEQ ID NO: 174)
miR-142-3p bs = UCCAUAAAGUAGGAAACACUACA
(SEQ ID NO: 152)
miR-126 bs = CGCAUUAUUACUCACGGUACGA

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); and (c) a 3′-UTR (e.g., as described herein).

In an aspect, an LNP composition comprising a polynucleotide comprising an open reading frame encoding a G6PC polypeptide (e.g., SEQ ID NO: 1) and comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a G6PC-related disease, disorder, or condition, e.g., GSD-Ia in a subject.

In an aspect, an LNP composition comprising a polynucleotide disclosed herein encoding a G6PC polypeptide, e.g., as described herein, can be administered with an additional agent, e.g., as described herein.

### Regions Having a 5′ Cap

The disclosure also includes a polynucleotide that comprises both a 5′ Cap and a polynucleotide of the present invention (e.g., a polynucleotide comprising a nucleotide sequence encoding a G6PC polypeptide to be expressed).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a G6PC polypeptide) incorporate a cap moiety.

In some embodiments, polynucleotides of the present invention comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

Polynucleotides of the invention can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N1pN2p (cap 0), 7mG(5′)ppp(5′)N1mpNp (cap 1), and 7mG(5′)-ppp(5′)N1mpN2mp (cap 2).

As a non-limiting example, capping chimeric polynucleotides post-manufacture can be more efficient as nearly 100% of the chimeric polynucleotides can be capped. This is in contrast to −80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

As used here the term “cap” includes the inverted G nucleotide and can comprise one or more additional nucleotides 3′ of the inverted G nucleotide, e.g., 1, 2, 3, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group.

In one embodiment, a cap comprises a compound of formula (I)

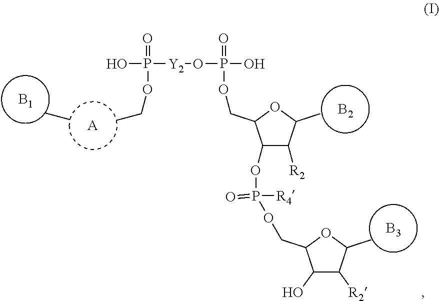

or a stereoisomer, tautomer or salt thereof, wherein

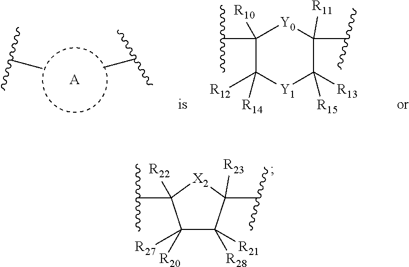

ring B₁ is a modified or unmodified Guanine;

ring B₂ and ring B₃ each independently is a nucleobase or a modified nucleobase;

X₂ is O, S(O)ₚ, NR₂₄ or CR₂₅R₂₆ in which p is 0, 1, or 2;

Y₀ is O or CR₆R₇;

Y1 is O, S(O)ₙ, CR₆R₇, or NR₈, in which n is 0, 1, or 2;

each - - - is a single bond or absent, wherein when each - - - is a single bond, Yi is O, S(O)ₙ, CR₆R₇, or NR₈; and when each - - - is absent, Yi is void;

Y₂ is (OP(O)R₄)m in which m is 0, 1, or 2, or —O—(CR₄₀R₄₁)ₜ₁-Q₀-(CR₄₂R₄₃)v-, in which Q₀ is a bond, O, S(O)r, NR₄₄, or CR₄₅R₄₆, r is 0, 1, or 2, and each of u and v independently is 1, 2, 3 or 4;

each R₂ and R₂′ independently is halo, LNA, or OR₃;

each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

each R₄ and R₄′ independently is H, halo, C₁-C₆ alkyl, OH, SH, SeH, or BH₃⁻;

each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;

each of R₁₀, R₁₁, R₁₂, R₁₃R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,

each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
each of R₂₄, R₂₅, and R₂₆ independently is H or C₁-C₆ alkyl;
each of R₂₇ and R₂₈ independently is H or OR₂₉; or R₂₇ and R₂₈ together form 0-R₃₀-0; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl; R₃₀ is C₁-C₆ alkylene optionally substituted with one or more of halo, OH and

C₁-C₆ alkoxyl;

each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;

each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;

R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and each of R₄₇ and R₄₈, independently is H, halo, C₁-C₆ alkyl, OH, SH, SeH, or BH₃

It should be understood that a cap analog, as provided herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a cap comprises the following structure:

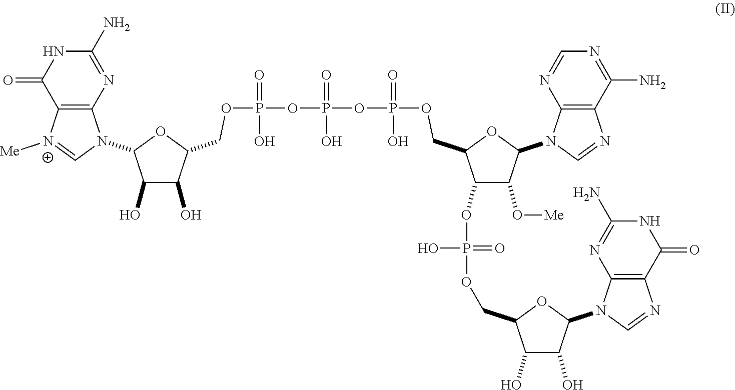

In other embodiments, a cap comprises the following structure:

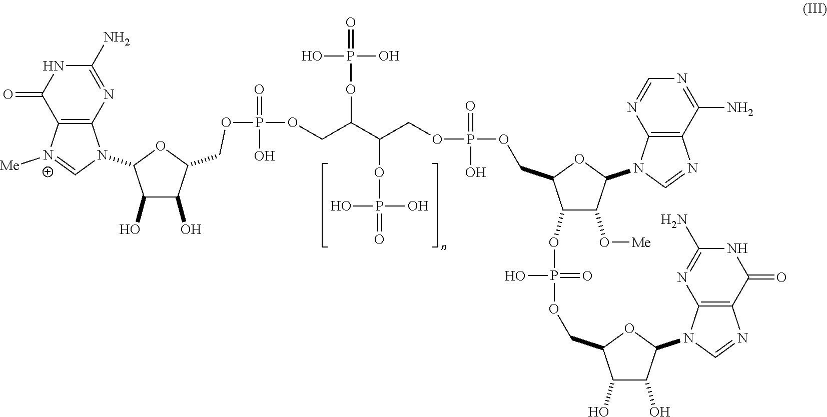

In yet other embodiments, a cap comprises the following structure:

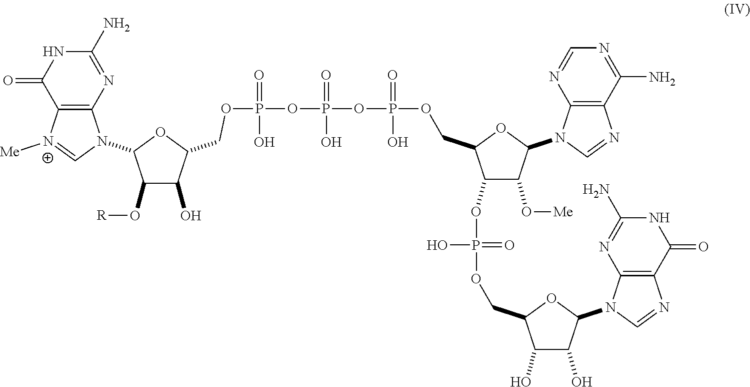

In still other embodiments, a cap comprises the following structure:

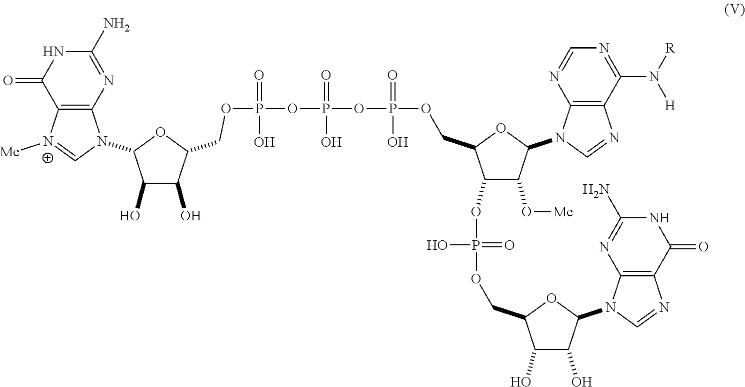

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

In some embodiments, a cap comprises a sequence selected from the following sequences: GAA, GAC, GAG, GAU, GCA, GCC, GCG, GCU, GGA, GGC, GGG, GGU, GUA, GUC, GUG, and GUU. In some embodiments, a cap comprises GAA. In some embodiments, a cap comprises GAC. In some embodiments, a cap comprises GAG. In some embodiments, a cap comprises GAU. In some embodiments, a cap comprises GCA. In some embodiments, a cap comprises GCC. In some embodiments, a cap comprises GCG. In some embodiments, a cap comprises GCU. In some embodiments, a cap comprises GGA. In some embodiments, a cap comprises GGC. In some embodiments, a cap comprises GGG. In some embodiments, a cap comprises GGU. In some embodiments, a cap comprises GUA. In some embodiments, a cap comprises GUC. In some embodiments, a cap comprises GUG. In some embodiments, a cap comprises GUU.

In some embodiments, a cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a cap comprises m⁷GpppApA. In some embodiments, a cap comprises m⁷GpppApC. In some embodiments, a cap comprises m⁷GpppApG. In some embodiments, a cap comprises m⁷GpppApU. In some embodiments, a cap comprises m⁷GpppCpA. In some embodiments, a cap comprises m⁷GpppCpC. In some embodiments, a cap comprises m⁷GpppCpG. In some embodiments, a cap comprises m⁷GpppCpU. In some embodiments, a cap comprises m⁷GpppGpA. In some embodiments, a cap comprises m⁷GpppGpC. In some embodiments, a cap comprises m⁷GpppGpG. In some embodiments, a cap comprises m⁷GpppGpU. In some embodiments, a cap comprises m⁷GpppUpA. In some embodiments, a cap comprises m⁷GpppUpC. In some embodiments, a cap comprises m⁷GpppUpG. In some embodiments, a cap comprises m⁷GpppUpU.

A cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a cap comprises m⁷G_{3′OMe}pppApA. In some embodiments, a cap comprises m⁷G_{3′OMe}pppApC. In some embodiments, a cap comprises m⁷G_{3′OMe}pppApG. In some embodiments, a cap comprises m⁷G_{3′OMe}pppApU. In some embodiments, a cap comprises m⁷G_{3′OMe}pppCpA. In some embodiments, a cap comprises m⁷G_{3′OMe}pppCpC. In some embodiments, a cap comprises m⁷G_{3′OMe}pppCpG. In some embodiments, a cap comprises m⁷G_{3′OMe}pppCpU. In some embodiments, a cap comprises m⁷G_{3′OMe}pppGpA. In some embodiments, a cap comprises m⁷G_{3′OMe}pppGpC. In some embodiments, a cap comprises m⁷G_{3′OMe}pppGpG. In some embodiments, a cap comprises m⁷G_{3′OMe}pppGpU. In some embodiments, a cap comprises m⁷G_{3′OMe}pppUpA. In some embodiments, a cap comprises m⁷G_{3′OMe}pppUpC. In some embodiments, a cap comprises m⁷G_{3′OMe}pppUpG. In some embodiments, a cap comprises m⁷G_{3′OMe}pppUpU.

A cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a cap comprises m⁷G_{3′OMe}pppA_{2′OMe}pA. In some embodiments, a cap comprises m⁷G_{3′OMe}pppA_{2′OMe}pC. In some embodiments, a cap comprises m⁷G_{3′OMe}pppA_{2′OMe}pG. In some embodiments, a cap comprises m⁷G_{3′OMe}pppA_{2′OMe}pU. In some embodiments, a cap comprises m⁷G_{3′OMe}pppC_{2′OMe}pA. In some embodiments, a cap comprises m⁷G_{3′OMe}pppC_{2′OMe}pC. In some embodiments, a cap comprises m⁷G_{3′OMe}pppC_{2′OMe}pG. In some embodiments, a cap comprises m⁷G_{3′OMe}pppC_{2′OMe}pU. In some embodiments, a cap comprises m⁷G_{3′OMe}pppG_{2′OMe}pA. In some embodiments, a cap comprises m⁷G_{3′OMe}pppG_{2′OMe}pC. In some embodiments, a cap comprises m⁷G_{3′OMe}pppG_{2′OMe}pG. In some embodiments, a cap comprises m⁷G_{3′OMe}pppG_{2′OMe}pU. In some embodiments, a cap comprises m⁷G_{3′OMe}pppU_{2′OMe}pA. In some embodiments, a cap comprises m⁷G_{3′OMe}pppU_{2′OMe}pC. In some embodiments, a cap comprises m⁷G_{3′OMe}pppU_{2′OMe}pG. In some embodiments, a cap comprises m⁷ G_{3′OMe}pppU_{2′OMe}pU.

A cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a cap comprises m⁷GpppA_{2′OMe}pA. In some embodiments, a cap comprises m⁷GpppA_{2′OMe}pC. In some embodiments, a cap comprises m⁷GpppA_{2′OMe}pG. In some embodiments, a cap comprises m⁷GpppA_{2′OMe}pU. In some embodiments, a cap comprises m⁷GpppC_{2′OMe}pA. In some embodiments, a cap comprises m⁷GpppC_{2′OMe}pC. In some embodiments, a cap comprises m⁷GpppC_{2′OMe}pG. In some embodiments, a trinucleotide cap comprises m⁷GpppC_{2′OMe}pU. In some embodiments, a cap comprises m⁷GpppG_{2′OMe}pA. In some embodiments, a cap comprises m⁷GpppG_{2′OMe}pC. In some embodiments, a cap comprises m⁷GpppG_{2′OMe}pG. In some embodiments, a cap comprises m⁷GpppG_{2′OMe}pU. In some embodiments, a cap comprises m⁷GpppU_{2′OMe}pA. In some embodiments, a cap comprises m⁷GpppU_{2′OMe}pC. In some embodiments, a cap comprises m⁷GpppU_{2′OMe}pG. In some embodiments, a cap comprises m⁷GpppU_{2′OMe}pU.

In some embodiments, a cap comprises m⁷Gpppm⁶A_{2′OMe}pG. In some embodiments, a cap comprises m⁷Gpppe⁶A_{2′OMe}pG.

In some embodiments, a cap comprises GAG. In some embodiments, a cap comprises GCG. In some embodiments, a cap comprises GUG. In some embodiments, a cap comprises GGG.

In some embodiments, a cap comprises any one of the following structures:

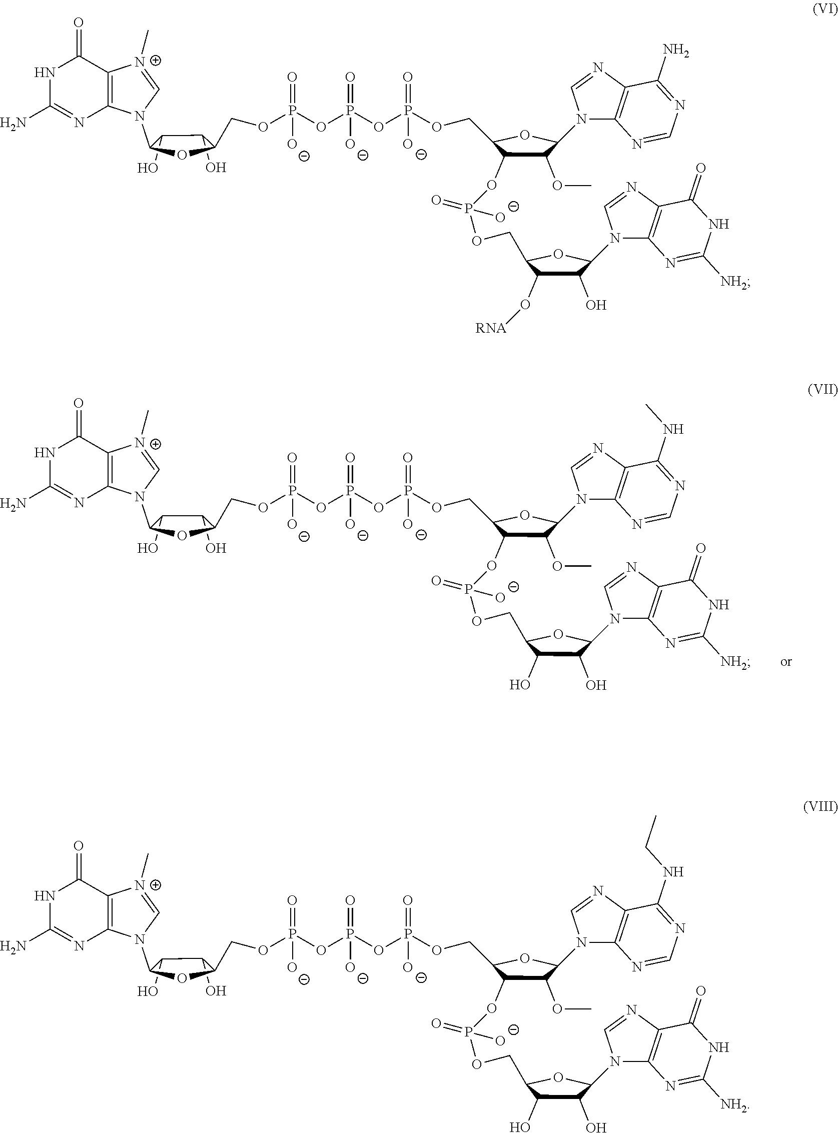

In some embodiments, the cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the cap comprises the following structure:

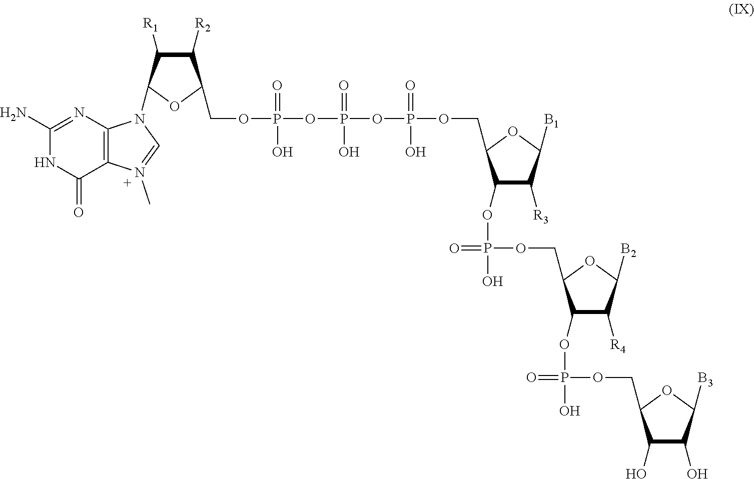

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N₆-methyladenine. In some embodiments, B₁ is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a cap comprises GGAG. In some embodiments, a cap comprises the following structure:

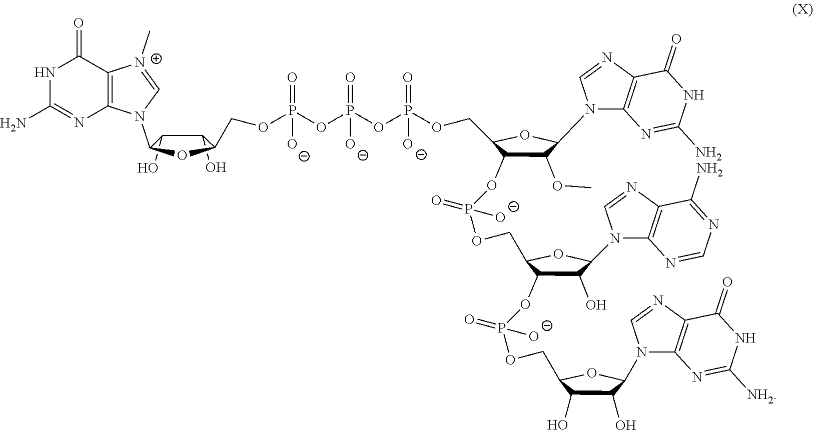

### Poly-A Tails

In some embodiments, the polynucleotides of the present disclosure (e.g., a polynucleotide comprising a nucleotide sequence encoding a G6PC polypeptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:195).

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:196).

In some embodiments, the polyA tail comprises an alternative nucleoside, e.g., inverted thymidine. PolyA tails comprising an alternative nucleoside, e.g., inverted thymidine, may be generated as described herein. For instance, mRNA constructs may be modified by ligation to stabilize the poly(A) tail. Ligation may be performed using 0.5-1.5 mg/mL mRNA (5′ Cap1, 3′ A100), 50 mM Tris-HCl pH 7.5, 10 mM MgCl₂, 1 mM TCEP, 1000 units/mL T₄ RNA Ligase 1, 1 mM ATP, 20% w/v polyethylene glycol 8000, and 5:1 molar ratio of modifying oligo to mRNA. Modifying oligo has a sequence of 5′-phosphate-AAAAAAAAAAAAAAAAAAAA-(inverted deoxythymidine (idT) (SEQ ID NO:209)) (see below). Ligation reactions are mixed and incubated at room temperature (˜22° C.) for, e.g., 4 hours. Stable tail mRNA are purified by, e.g., dT purification, reverse phase purification, hydroxyapatite purification, ultrafiltration into water, and sterile filtration. The resulting stable tail-containing mRNAs contain the following structure at the 3′end, starting with the polyA region:

(SEQ ID NO: 211)
A₁₀₀-UCUAGAAAAAAAAAAAAAAAAAAAA-inverted
deoxythymidine.

Modifying oligo to stabilize tail (5′-phosphate-AAAAAAAAAAAAAAAAAAAA-(inverted deoxythymidine)(SEQ ID NO:209)):

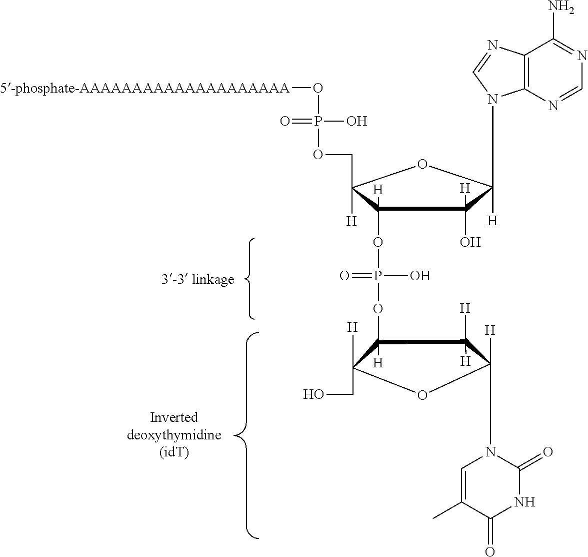

In some instances, the polyA tail comprises A100-UCUAG-A20-inverted deoxy-thymidine (SEQ ID NO:211). In some instances, the polyA tail consists of A100-UCUAG-A20-inverted deoxy-thymidine (SEQ ID NO:211).

Combination of mRNA Elements

Any of the polynucleotides disclosed herein can comprise one, two, three, or all of the following elements: (a) a 5′-UTR, e.g., as described herein; (b) a coding region comprising a stop element (e.g., as described herein); (c) a 3′-UTR (e.g., as described herein) and; optionally (d) a 3′ stabilizing region, e.g., as described herein. Also disclosed herein are LNP compositions comprising the same.

In an embodiment, a polynucleotide of the disclosure comprises (a) a 5′ UTR described in Table 1 or a variant or fragment thereof and (b) a coding region comprising a stop element provided herein. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (a) a 5′ UTR described in Table 1 or a variant or fragment thereof and (c) a 3′ UTR described in Table 2 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (c) a 3′ UTR described in Table 2 or a variant or fragment thereof and (b) a coding region comprising a stop element provided herein. In an embodiment, the polynucleotide comprises a sequence provided in Table 3. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises (a) a 5′ UTR described in Table 1 or a variant or fragment thereof; (b) a coding region comprising a stop element provided herein; and (c) a 3′ UTR described in Table 2 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

TABLE 3
Exemplary 3′ UTR and stop element sequences

| | Sequence | |
|---|---|---|
| SEQ ID NO | information | Sequence |
| 121 | 3′ UTR with stop | UAGGGUUAAGCUGGAGCCUCGGUGGCCUAGCUUCUUGCCC |
| | C11 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 122 | 3′ UTR with stop | UAAAGCUCCGCUGGAGCCUCGGUGGCCUAGCUUCUUGCCC |
| | C10 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 123 | 3′ UTR with stop | UAAGCCCCUGCUGGAGCCUCGGUGGCCUAGCUUCUUGCCC |
| | C9 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 124 | 3′ UTR with stop | UAAGCACCCGCUGGAGCCUCGGUGGCCUAGCUUCUUGCCC |
| | C8 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 125 | 3′ UTR with stop | UAAGCCCCUCCGGGGGCCUCGGUGGCCUAGCUUCUUGCCC |
| | C7 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 126 | 3′ UTR with stop | UAAGGCUAAGCUGGAGCCUCGGUGGCCUAGCUUCUUGCCC |
| | C6 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 127 | 3′ UTR with stop | UAAGUCUCCGCUGGAGCCUCGGUGGCCUAGCUUCUUGCCC |
| | C5 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 128 | 3′ UTR with stop | UAAAGCUAAGCUGGAGCCUCGGUGGCCUAGCUUCUUGCCC |
| | C4 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 129 | 3′ UTR with stop | UAAGUCUAAGCUGGAGCCUCGGUGGCCUAGCUUCUUGCCC |
| | C3 (underlined) | CUUGGGCCUCCCCCCAGCCCCUCCUCCCCUUCCUGCACCC |
| | | GUACCCCCGUGGUCUUUGAAUAAAGUCUGAGUGGGCGGC |
| 130 | 3′ UTR with CIO | UAAAGCUCCAUAAAGUAGGAAACACUACAGCUGGAGCCUC |
| | stop (underlined) | GGUGGCCUAGCUUCUUGCCCCUUGGGCCUCCAUAAAGUAG |
| | | GAAACACUACAUCCCCCCAGCCCCUCCUCCCCUUCCUGCA |
| | | CCCGUACCCCCUCCAUAAAGUAGGAAACACUACAGUGGUC |
| | | UUUGAAUAAAGUCUGAGUGGGCGGC |
| 131 | 3′ UTR with C7 | UAAGCCCCUCCGGGGUCCAUAAAGUAGGAAACACUACAGC |
| | stop (underlined) | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |
| 132 | 3′ UTR with C8 | UAAAGCUCCCCGGGGUCCAUAAAGUAGGAAACACUACAGC |
| | stop (underlined) | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |

Methods of Making mRNAs

The present disclosure also provides methods for making a mRNA of the invention or a complement thereof.

In some aspects, a mRNA disclosed herein can be constructed using in vitro transcription (IVT). In other aspects, a mRNA disclosed herein can be constructed by chemical synthesis using an oligonucleotide synthesizer.

In other aspects, a mRNA disclosed herein is made by using a host cell. In certain aspects, a mRNA disclosed herein is made by one or more combination of the in vitro transcription (IVT), chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized mRNA. The resultant mRNAs can then be examined for their ability to produce protein and/or produce a therapeutic outcome.

### Lipid Nanoparticle Formulations

In some embodiments, nucleic acids of the invention are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2019/052009, PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG- lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Amino Lipids

In some aspects, the amino lipids of the present disclosure may be one or more of compounds of Formula (L-VI):

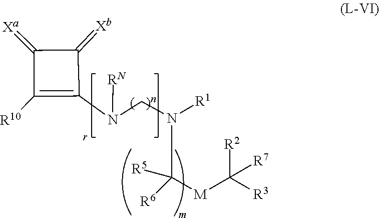

### or its N-oxide,

or a salt or isomer thereof, wherein
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
each R is independently selected from the group consisting of H, C₁₋₃ alkyl, and C₂₋₃ alkenyl;
R^{N} is H, or C₁₋₃ alkyl;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I;
X^{a} and X^{b} are each independently O or S;
   R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, —N(R)-carbocycle, —N(R)-heterocycle, —N(R)-aryl, —N(R)-heteroaryl, —N(R)(CH₂)ₜ₁-carbocycle, —N(R)(CH₂)ₜ₁-heterocycle, —N(R)(CH₂)ₜ₁-aryl, —N(R)(CH₂)ₜ₁-heteroaryl, a carbocycle, a heterocycle, aryl and heteroaryl;
   m is selected from 5, 6, 7, 8, 9, 10, 11, 12, and 13;
   n is selected from 1, 2, 3, 4, 5, 6, 7, 8, 9, and 10;
   r is 0 or 1;
   t¹ is selected from 1, 2, 3, 4, and 5;
   p¹ is selected from 1, 2, 3, 4, and 5;
   q¹ is selected from 1, 2, 3, 4, and 5; and
   s¹ is selected from 1, 2, 3, 4, and 5.

In some embodiments, a subset of compounds of Formula (L-VI) includes those of Formula (L-VI-a):

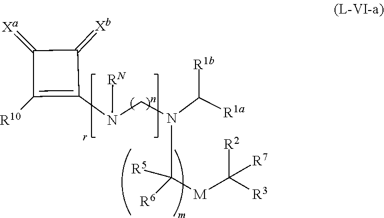

### or its N-oxide,

or a salt or isomer thereof, wherein
R^{1a} and R^{1b} are independently selected from the group consisting of C₁₋₁₄ alkyl and C₂-i4 alkenyl; and
R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In other embodiments, a subset of compounds of Formula (L-VI) includes those of Formula (L-VII):

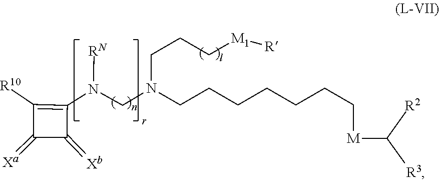

or its N-oxide, or a salt or isomer thereof, wherein
1 is selected from 1, 2, 3, 4, and 5;
M₁ is a bond or M′; and
R² and R³ are independently selected from the group consisting of H, C₁₋₁₄ alkyl, and C₂₋₁₄ alkenyl.

In other embodiments, a subset of compounds of Formula (L-VI) includes those of Formula (L-VIII):

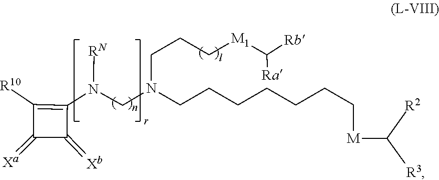

or its N-oxide, or a salt or isomer thereof, wherein
1 is selected from 1, 2, 3, 4, and 5;
M₁ is a bond or M′; and
R^{a′} and R^{b′} are independently selected from the group consisting of C₁₋₁₄ alkyl and C₂₋₁₄ alkenyl; and
R² and R³ are independently selected from the group consisting of C₁₋₁₄ alkyl, and C₂₋₁₄ alkenyl.

The compounds of any one of the Formulae herein include one or more of the following features when applicable.

In some embodiments, M₁ is M′.

In some embodiments, M and M′ are independently —C(O)O— or —OC(O)—.

In some embodiments, at least one of M and M′ is —C(O)O— or —OC(O)—.

In certain embodiments, at least one of M and M′ is —OC(O)—.

In certain embodiments, M is —OC(O)— and M′ is —C(O)O—. In some embodiments, M is —C(O)O— and M′ is —OC(O)—. In certain embodiments, M and M′ are each —OC(O)—. In some embodiments, M and M′ are each —C(O)O—.

In certain embodiments, at least one of M and M′ is —OC(O)-M″-C(O)O—.

In some embodiments, M and M′ are independently —S—S—.

In some embodiments, at least one of M and M′ is —S—S.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″-C(O)O—, in which M″ is a bond, C₁₋₁₃ alkyl or C₂₋₁₃ alkenyl. In other embodiments, M″ is C₁₋₆ alkyl or C₂₋₆ alkenyl. In certain embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, 1 is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁶ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R^{x} is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)ᵥN(R)₂, v is 1, 2 or 3 and each R is H or methyl. For example, R^{x} is methanamino, methylmethanamino, or dimethylmethanamino. For example, R^{x} is aminomethanyl, methylaminomethanyl, or dimethylaminomethanyl. For example, R^{x} is aminoethanyl, methylaminoethanyl, or dimethylaminoethanyl. For example, R^{x} is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R¹ is different from —(CHR⁵R⁶)ₘ-M-CR²R³R⁷.

In some embodiments, —CHR^{1a}R^{1b} is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, R^{b′} is C₁₋₁₄ alkyl. In some embodiments, R^{b′} is C₂₋₁₄ alkyl. In some embodiments, R^{b′} is C₃₋₁₄ alkyl. In some embodiments, R^{b′} is C₁₋₈ alkyl. In some embodiments, R^{b′} is C₁₋₈ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In other embodiments, a subset of compounds of Formula (L-VI) includes those of Formula (L-VIIa):

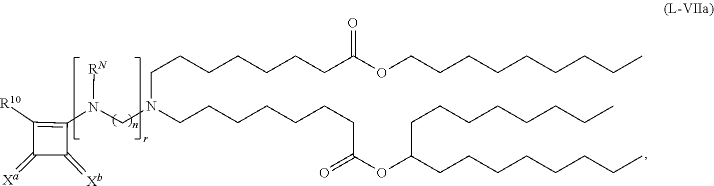

or its N-oxide, or a salt or isomer thereof.

In other embodiments, a subset of compounds of Formula (L-VI) includes those of Formula (L-VIIIa):

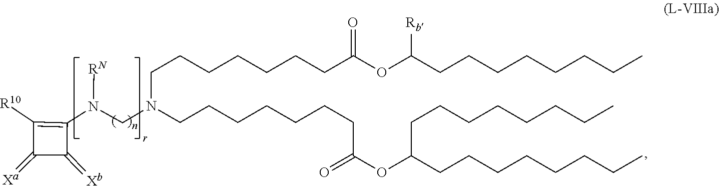

or its N-oxide, or a salt or isomer thereof.

In other embodiments, a subset of compounds of Formula (L-VI) includes those of Formula (L-VIIIb):

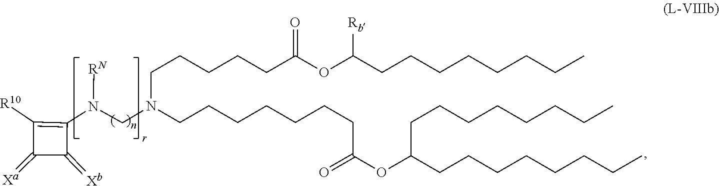

or its N-oxide, or a salt or isomer thereof.

In other embodiments, a subset of compounds of Formula (L-VI) includes those of Formula (L-VIIb-1):

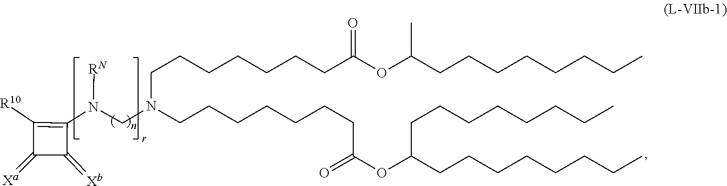

or its N-oxide, or a salt or isomer thereof.

In other embodiments, a subset of compounds of Formula (L-VI) includes those of Formula (L-VIIb-2):

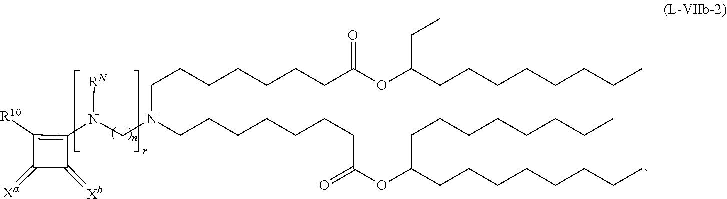

or its N-oxide, or a salt or isomer thereof.

In other embodiments, a subset of compounds of Formula (L-VI) includes those of Formula (L-VIIb-3):

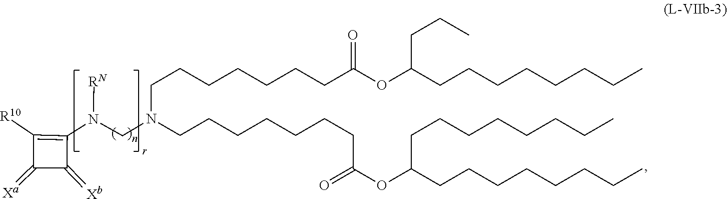

or its N-oxide, or a salt or isomer thereof.

In other embodiments, a subset of compounds of Formula (L-VI) includes those of Formula (L-VIIb-4):

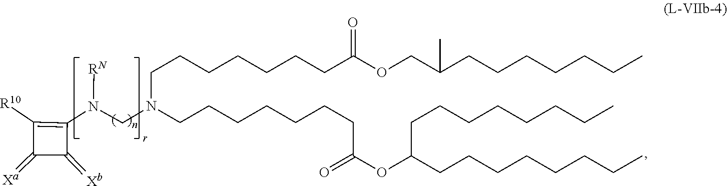

or its N-oxide, or a salt or isomer thereof.

In other embodiments, a subset of compounds of Formula (L-VI) includes those of Formula (L-VIIc):

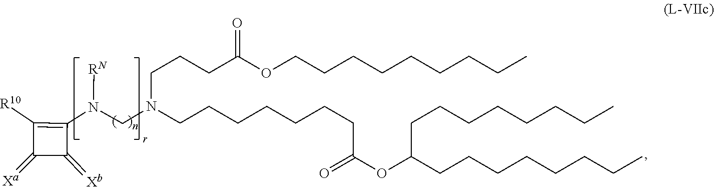

or its N-oxide, or a salt or isomer thereof.

In other embodiments, a subset of compounds of Formula (L-VI) includes those of Formula (L-VIId):

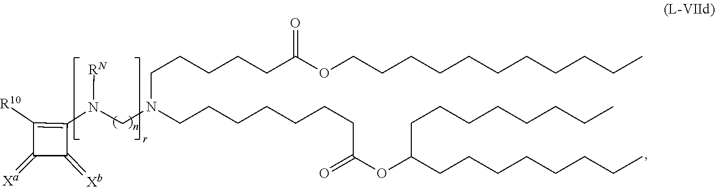

or its N-oxide, or a salt or isomer thereof.

In other embodiments, a subset of compounds of Formula (L-VI) includes those of Formula (L-VIIIc):

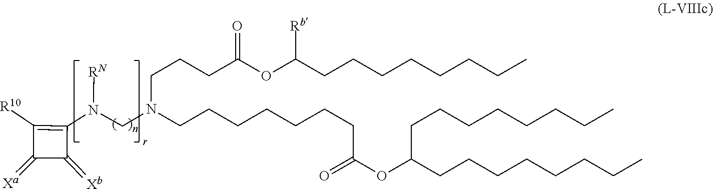

or its N-oxide, or a salt or isomer thereof.

In other embodiments, a subset of compounds of Formula (L-VI) includes those of Formula (L-VIIId):

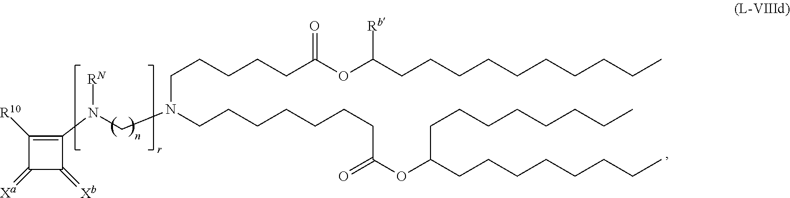

or its N-oxide, or a salt or isomer thereof.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

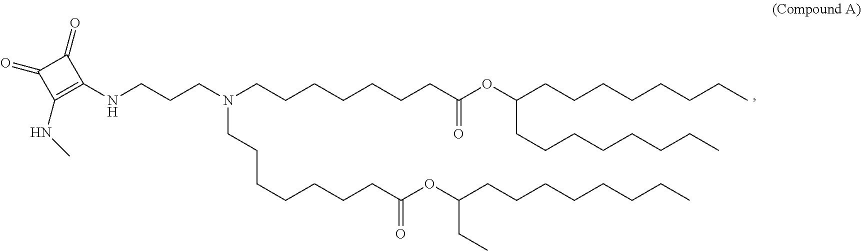 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or its N-oxide, or a salt or isomer thereof.

The central amine moiety of a lipid according to any Formulae herein may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

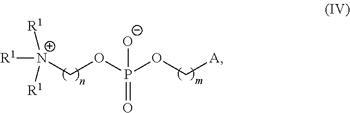

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

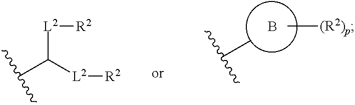

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), —C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(s)N(RN), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

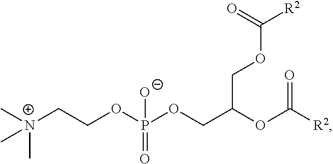

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

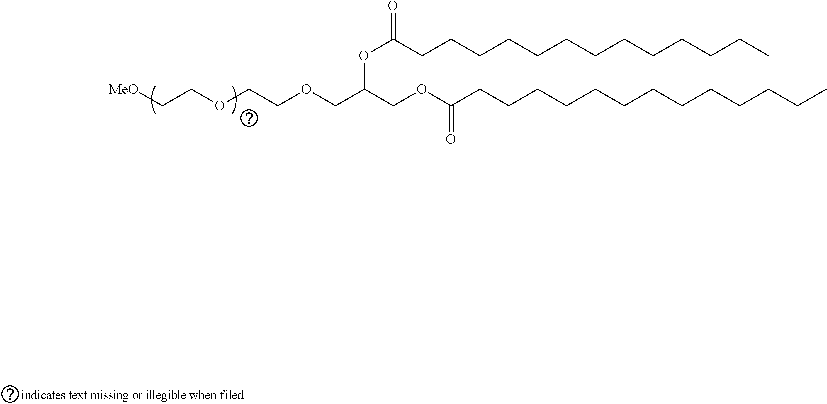

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

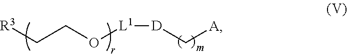

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

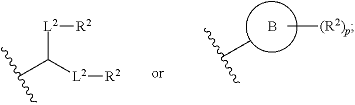

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), —C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Fomula (V) is a PEG-OH lipid (i.e., R³ is —OR^{O}, and R^{O} is hydrogen). In certain embodiments, the compound of Formula (V) is of Formula (V-OH):

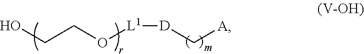

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

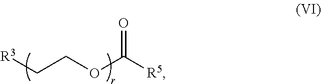

or a salt thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI-OH):

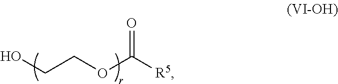

or a salt thereof. In some embodiments, r is 40-50.

In yet other embodiments the compound of Formula (VI) is:

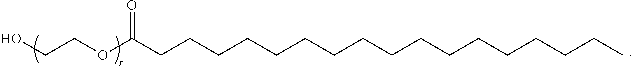

or a salt thereof.

In one embodiment, the compound of Formula (VI) is

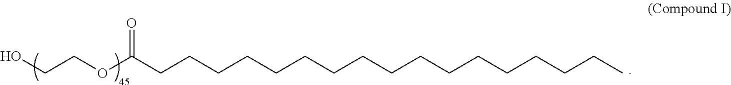

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, a LNP of the invention comprises an amino lipid of any of Formula I, II or III, a phospholipid comprising DSPC, a structural lipid, and a PEG lipid comprising PEG-DMG.

In some embodiments, a LNP of the invention comprises an amino lipid of any of Formula I, II or III, a phospholipid comprising DSPC, a structural lipid, and a PEG lipid comprising a compound having Formula VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid having Formula IV, a structural lipid, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

### Methods of Use

The mRNAs and lipid nanoparticles described herein are used in the preparation, manufacture and therapeutic use of to treat and/or prevent G6PC-related diseases, disorders or conditions, e.g., GSD-Ia. In some embodiments, the mRNAs and lipid nanoparticles described herein are used to treat and/or prevent resulting from defective G6PC or deficient G6PC. In some embodiments, the mRNAs and lipid nanoparticles described herein are used to improve glycemia or improve blood glucose homeostasis resulting from defective G6PC or deficient G6PC. In some embodiments, the mRNAs and lipid nanoparticles described herein are used to treat and/or prevent the excessive accumulation of glycogen resulting from defective or deficient G6PC. In some embodiments, the mRNAs and lipid nanoparticles described herein are used to treat and/or prevent hepatomegaly, nephromegaly, hypercholesterolemia, hypertriglyceridemia, hyperuricemia, and/or lactic academia in a subject with defective or deficient G6PC, e.g., a subject with GSD-Ia. In some embodiments, the mRNAs and lipid nanoparticles described herein are used to treat and/or prevent liver adenoma (e.g., hepatocellular adenoma) in a subject (e.g., human) in need thereof. In some embodiments, the mRNAs and lipid nanoparticles described herein are used to treat and/or prevent liver carcinoma (e.g., hepatocellular carcinoma) in a subject (e.g., human) in need thereof. In some embodiments, the mRNAs and lipid nanoparticles described herein are used to treat a liver tumor in a subject (e.g., human) in need thereof. In some embodiments, the mRNAs and lipid nanoparticles described herein are used to prevent liver tumor formation in a subject (e.g., human) in need thereof. In some embodiments, the mRNAs and lipid nanoparticles described herein are used to treat and/or prevent hepatocellular adenoma and/or hepatocellular carcinoma.

In some embodiments, the mRNAs and lipid nanoparticles described herein are used in methods for reducing the levels of glycogen in a subject in need thereof, e.g., a subject having GSD-Ia that is fasting, e.g., a subject who has not had a meal. In some embodiments, the mRNAs and lipid nanoparticles described herein are used in methods for increasing the levels of glucose (e.g., increasing blood glucose levels) in a subject in need thereof, e.g., a subject having GSD-Ia that is fasting, e.g., a subject who has not had a meal. For instance, one aspect of the invention provides a method of alleviating the symptoms of GSD-Ia in a subject (e.g., a subject with GSD-Ia that is fasting) comprising the administration of a lipid nanoparticle described herein. In some embodiments, the subject with GSD-Ia has been fasting for 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, or 24 or more hours. In some embodiments, the subject with GSD-Ia has been fasting for 1 to 24 hours, 1 to 12 hours, 2 to 24 hours, 2 to 12 hours, 3 to 12 hours, 4 to 10 hours, 6 to 24 hours, 6 to 12 hours, or 5 to 18 hours. In some embodiments, the subject has been fasting for more than one day, e.g., 1, 2, or 3 days.

In some embodiments, the mRNAs and lipid nanoparticles described herein are used to reduce the level of glycogen, the method comprising administering to the subject an effective amount of a polynucleotide encoding a G6PC polypeptide. In some embodiments, the administration of the mRNAs and lipid nanoparticles described herein results in a meaningful reduction in the level of glycogen (e.g., a statistically significant reduction in glycogen relative to the level of glycogen in the subject prior to administration), within a short period of time (e.g., within about 1 hour, within about 2 hours, within about 4 hours, within about 6 hours, within about 8 hours, within about 12 hours, within about 16 hours, within about 20 hours, or within about 24 hours) after administration of the polynucleotide, pharmaceutical composition or formulation of the invention.

In some embodiments, the mRNAs and lipid nanoparticles described herein are used to reduce the level of glucose-6-phosphate (G6P), the method comprising administering to the subject an effective amount of a polynucleotide encoding a G6PC polypeptide. In some embodiments, the G6PC encoded by the polynucleotide hydrolyzes the G6P to glucose and phosphate. In some embodiments, the administration of the mRNAs and lipid nanoparticles described herein results in a meaningful reduction in the level of glucose-6-phosphate (e.g., a statistically significant reduction in glucose-6-phosphate relative to the level of glucose-6-phosphate in the subject prior to administration), within a short period of time (e.g., within about 1 hour, within about 2 hours, within about 4 hours, within about 6 hours, within about 8 hours, within about 12 hours, within about 16 hours, within about 20 hours, or within about 24 hours) after administration of the polynucleotide, pharmaceutical composition or formulation of the invention.

In some embodiments, the mRNAs and lipid nanoparticles described herein are used to increase the level of glucose, the method comprising administering to the subject an effective amount of a polynucleotide encoding a G6PC polypeptide. In some embodiments, the mRNAs and lipid nanoparticles described herein increases or improves blood glucose homeostasis in the subject, e.g., when the subject is fasted or has not eaten. In some embodiments, the administration of the mRNAs and lipid nanoparticles described herein results in an increase in the level of blood glucose to at least 70 mg/dl (e.g., at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, or 150 mg/dl), within a short period of time (e.g., within about 1 hour, within about 2 hours, within about 4 hours, within about 6 hours, within about 8 hours, within about 12 hours, within about 16 hours, within about 20 hours, or within about 24 hours) after administration of the polynucleotide, pharmaceutical composition or formulation of the invention. In some embodiments, the administration of mRNAs and lipid nanoparticles described herein results in an increase in the level of glucose to at least 3.5 mmol/1 (e.g., at least 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.7, 6.8, 6.9, 7.0, 7.1, 7.2, 7.3, 7.4, 7.5, 7.6, 7.7, 7.8, 7.9, 8.0, or 8.5 mmol/1), within a short period of time (e.g., within about 1 hour, within about 2 hours, within about 4 hours, within about 6 hours, within about 8 hours, within about 12 hours, within about 16 hours, within about 20 hours, or within about 24 hours) after administration of the polynucleotide, pharmaceutical composition or formulation of the invention. For instance, one aspect of the invention provides a method of increasing the blood glucose levels in a subject with GSD-Ia that is fasting, comprising the administration of a lipid nanoparticle described herein. In some embodiments, administering the composition or formulation alleviates a symptom of GSD-Ia. In some embodiments, administering the lipid nanoparticle alleviates a symptom of GSD-Ia, e.g., improves glycemia or blood glucose homeostasis in the subject.

In some embodiments, the administration of an effective amount of a lipid nanoparticle described herein reduces the levels of a biomarker of GSD-Ia. In some embodiments, the administration of the lipid nanoparticle results in reduction in the level of one or more biomarkers of GSD-Ia, within a short period of time (e.g., within about 1 hour, within about 2 hours, within about 4 hours, within 6 hours, within about 8 hours, within about 12 hours, within about 16 hours, within about 20 hours, or within about 24 hours) after administration of the polynucleotide, pharmaceutical composition or formulation of the invention.

In some embodiments, the administration of an effective amount of a lipid nanoparticle described herein increases the levels of a biomarker of GSD-Ia. In some embodiments, the administration of the lipid nanoparticle results in an increase in the level of one or more biomarkers of GSD-Ia, within a short period of time (e.g., within about 1 hour, within about 2 hours, within about 4 hours, within 6 hours, within about 8 hours, within about 12 hours, within about 16 hours, within about 20 hours, or within about 24 hours) after administration of the lipid nanoparticle.

In some embodiments, the administration of a lipid nanoparticle described herein to a subject results in a decrease in glycogen to a level at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% lower than the level observed prior to the administration of the lipid nanoparticle. In some embodiments, the administration of a lipid nanoparticle described herein to a subject results in a decrease in glucose-6-phosphate (G6P) to a level at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% lower than the level observed prior to the administration of the lipid nanoparticle. In some embodiments, the decrease in glycogen and/or G6P is in hepatic cells or tissue. In some embodiments, the decrease in glycogen and/or G6P is in blood, plasma or serum.

In some embodiments, the administration of a lipid nanoparticle described herein to a subject results in an increase in glucose to a level at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% lower than the level observed prior to the administration of the lipid nanoparticle. In some embodiments, the increase in glucose is in hepatic cells or tissue. In some embodiments, the increase in glucose is in blood, plasma or serum.

In some embodiments, the administration of a lipid nanoparticle described herein results in expression of G6PC in cells of the subject. In some embodiments, administering the lipid nanoparticle results in an increase of G6PC expression and/or enzymatic activity in the subject. For example, in some embodiments, a lipid nanoparticle described herein are used in methods of administering a composition or formulation comprising an mRNA encoding a G6PC polypeptide to a subject, wherein the method results in an increase of G6PC expression and/or enzymatic activity in at least some cells of a subject.

In some embodiments, the administration of a lipid nanoparticle described herein to a subject results in an increase of G6PC expression and/or enzymatic activity in cells subject to a level at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% or more of the expression and/or activity level expected in a normal subject, e.g., a human not suffering from GSD-Ia. In some embodiments, the administration of a lipid nanoparticle described herein to a subject results in an increase of 1%, 2%, 3%, 4%, 5%, 6%, 7%, 8%, 9%, 10%, 15%, 20%, 25%, 30%, 35%, 40%, 45%, 505%, 55%, 60%, 65%, 70%, 75%, 80%, 85%, 90%, or 95% or more of G6PC expression and/or enzymatic activity in cells of a subject, e.g., a human subject with GSD-Ia.

In some embodiments, the administration of a lipid nanoparticle described herein results in expression of G6PC protein in at least some of the cells of a subject that persists for a period of time sufficient to allow significant glucose production to occur. In some embodiments, the administration of the lipid nanoparticle results in expression of G6PC protein in at least some of the cells of a subject that persists for a period of time sufficient to allow significant amounts of hydrolysis of glucose-6-phosphate (G6P) to glucose and phosphate to occur.

In some embodiments, the administration of a lipid nanoparticle described herein results in treatment and/or prevention of liver adenoma (e.g., hepatocellular adenoma) in a subject (e.g., human) in need thereof. In some embodiments, the administration reduces the size of a liver tumor in the subject by at least 5%, at least 10%, at least 20%, at least 30%, at least 40%, at least 50%, at least 60%, at least 70%, at least 80%, at least 90%, at least 95% or 100% compared to the size of the liver tumor in the subject a period of time (e.g., 1 day, 1 week, 1 month, 2 months, 3 months, 4 months, 5 months, 6, months, or 1 year) prior to administration of the lipid nanoparticle. In some embodiments, the administration reduces the number of liver tumors in the subject by at least 5%, at least 10%, at least 20%, at least 30%, at least 40%, at least 50%, at least 60%, at least 70%, at least 80%, at least 90%, at least 95% or 100% compared to the number of liver tumors in the subject a period of time (e.g., 1 day, 1 week, 1 month, 2 months, 3 months, 4 months, 5 months, 6, months, or 1 year) prior to administration of the lipid nanoparticle. In some embodiments, the subject does not develop a liver adenoma within 1 day, 1 week, 1 month, 2 months, 3 months, 4 months, 5 months, 6, months, or 1 year of being administered a lipid nanoparticle described herein.

In some embodiments, the mRNAs and lipid nanoparticles described herein are used to treat and/or prevent liver carcinoma (e.g., hepatocellular carcinoma) in a subject (e.g., human) in need thereof. In some embodiments, the administration reduces the size of a tumor in the subject by at least 5%, at least 10%, at least 20%, at least 30%, at least 40%, at least 50%, at least 60%, at least 70%, at least 80%, at least 90%, at least 95% or 100% compared to the size of the tumor in the subject a period of time (e.g., 1 day, 1 week, 1 month, 2 months, 3 months, 4 months, 5 months, 6, months, or 1 year) prior to administration of the lipid nanoparticle. In some embodiments, the administration reduces the number of tumors in the subject by at least 5%, at least 10%, at least 20%, at least 30%, at least 40%, at least 50%, at least 60%, at least 70%, at least 80%, at least 90%, at least 95% or 100% compared to the number of tumors in the subject a period of time (e.g., 1 day, 1 week, 1 month, 2 months, 3 months, 4 months, 5 months, 6, months, or 1 year) prior to administration of the lipid nanoparticle. In some embodiments, the subject does not develop a liver carcinoma within 1 day, 1 week, 1 month, 2 months, 3 months, 4 months, 5 months, 6, months, or 1 year of being administered a lipid nanoparticle described herein.

In some embodiments, the mRNAs and lipid nanoparticles described herein are used to treat a liver tumor in a subject (e.g., human) in need thereof. In some embodiments, the administration reduces the size of a liver tumor in the subject by at least 5%, at least 10%, at least 20%, at least 30%, at least 40%, at least 50%, at least 60%, at least 70%, at least 80%, at least 90%, at least 95% or 100% compared to the size of the liver tumor in the subject a period of time (e.g., 1 day, 1 week, 1 month, 2 months, 3 months, 4 months, 5 months, 6, months, or 1 year) prior to administration of the lipid nanoparticle. In some embodiments, the administration reduces the number of liver tumors in the subject by at least 5%, at least 10%, at least 20%, at least 30%, at least 40%, at least 50%, at least 60%, at least 70%, at least 80%, at least 90%, at least 95% or 100% compared to the number of liver tumors in the subject a period of time (e.g., 1 day, 1 week, 1 month, 2 months, 3 months, 4 months, 5 months, 6, months, or 1 year) prior to administration of the lipid nanoparticle. In some embodiments, the subject does not develop a liver tumor within 1 day, 1 week, 1 month, 2 months, 3 months, 4 months, 5 months, 6, months, or 1 year of being administered a lipid nanoparticle described herein.

In some embodiments, the mRNAs and lipid nanoparticles described herein are used to prevent liver tumor formation in a subject (e.g., human) in need thereof. In some embodiments, the subject does not develop a liver tumor within 1 day, 1 week, 1 month, 2 months, 3 months, 4 months, 5 months, 6, months, or 1 year of being administered a lipid nanoparticle described herein.

### G6PC Protein Expression Levels

G6PC protein expression levels can be measured or determined by any art-recognized method for determining protein levels in biological samples, e.g., from blood or tissue samples or a needle biopsy. The term “level” or “level of a protein” as used herein, preferably means the weight, mass or concentration of the protein within a sample or a subject. It will be understood by the skilled artisan that in certain embodiments the sample may be subjected, e.g., to any of the following: purification, precipitation, separation, e.g. centrifugation and/or HPLC, and subsequently subjected to determining the level of the protein, e.g., using mass and/or spectrometric analysis. In exemplary embodiments, enzyme-linked immunosorbent assay (ELISA) can be used to determine protein expression levels. In other exemplary embodiments, protein purification, separation and LC-MS can be used as a means for determining the level of a protein according to the invention.

### G6PC Protein Activity

In subjects with GSD-Ia, G6PC enzymatic activity is reduced compared to a normal physiological activity level. Further aspects disclosed herein feature measurement, determination and/or monitoring of the activity level(s) (i.e., enzymatic activity level(s)) of G6PC protein in a subject, for example, in an animal (e.g., rodent, primate, and the like) or in a human subject. Activity levels can be measured or determined by any art-recognized method for determining enzymatic activity levels in biological samples. The term “activity level” or “enzymatic activity level” as used herein, preferably means the activity of the enzyme per volume, mass or weight of sample or total protein within a sample. In exemplary embodiments, the “activity level” or “enzymatic activity level” is described in terms of units per milliliter of fluid (e.g., bodily fluid, e.g., serum, plasma, urine and the like) or is described in terms of units per weight of tissue or per weight of protein (e.g., total protein) within a sample. Units (“U”) of enzyme activity can be described in terms of weight or mass of substrate hydrolyzed per unit time. In certain embodiments of the invention feature G6PC activity described in terms of U/ml plasma or U/mg protein (tissue), where units (“U”) are described in terms of nmol substrate hydrolyzed per hour (or nmol/hr).

In certain embodiments, an mRNA therapy of the invention features a lipid nanoparticle comprising a dose of mRNA effective to result in at least 5 U/mg, at least 10 U/mg, at least 20 U/mg, at least 30 U/mg, at least 40 U/mg, at least 50 U/mg, at least 60 U/mg, at least 70 U/mg, at least 80 U/mg, at least 90 U/mg, at least 100 U/mg, or at least 150 U/mg of G6PC activity in tissue (e.g., liver) between 2 and 6 hours, between 6 and 12 hours, or between 12 and 24, between 24 and 48, or between 48 and 72 hours post administration (e.g., at 48 or at 72 hours post administration).

In exemplary embodiments, an mRNA therapy described herein features a pharmaceutical composition comprising a single intravenous dose of mRNA that results in the above-described levels of activity. In another embodiment, an mRNA therapy of the invention features a pharmaceutical composition which can be administered in multiple single unit intravenous doses of mRNA that maintain the above-described levels of activity.

### GSD-Ia Biomarkers

Further aspects of the invention feature determining the level (or levels) of a biomarker determined in a sample as compared to a level (e.g., a reference level) of the same or another biomarker in another sample, e.g., from the same subject, from another subject, from a control and/or from the same or different time points, and/or a physiologic level, and/or an elevated level, and/or a supraphysiologic level, and/or a level of a control. The skilled artisan will be familiar with physiologic levels of biomarkers, for example, levels in normal or wildtype animals, normal or healthy subjects, and the like, in particular, the level or levels characteristic of subjects who are healthy and/or normal functioning. As used herein, the phrase “elevated level” means amounts greater than normally found in a normal or wildtype preclinical animal or in a normal or healthy subject, e.g. a human subject. As used herein, the term “supraphysiologic” means amounts greater than normally found in a normal or wildtype preclinical animal or in a normal or healthy subject, e.g. a human subject, optionally producing a significantly enhanced physiologic response. As used herein, the term “comparing” or “compared to” preferably means the mathematical comparison of the two or more values, e.g., of the levels of the biomarker(s). It will thus be readily-apparent to the skilled artisan whether one of the values is higher, lower or identical to another value or group of values if at least two of such values are compared with each other. Comparing or comparison to can be in the context, for example, of comparing to a control value, e.g., as compared to a reference blood, serum, plasma, and/or tissue (e.g., liver) biomarker level, in said subject prior to administration (e.g., in a person suffering from GSD-Ia) or in a normal or healthy subject. Comparing or comparison to can also be in the context, for example, of comparing to a control value, e.g., as compared to a reference blood, serum, plasma and/or tissue (e.g., liver) biomarker level in said subject prior to administration (e.g., in a person suffering from GSD-Ia) or in a normal or healthy subject. As used herein, a “control” is preferably a sample from a subject wherein the GSD-Ia status of said subject is known. In one embodiment, a control is a sample of a healthy patient. In another embodiment, the control is a sample from at least one subject having a known GSD-Ia status, for example, a severe, mild, or healthy GSD-Ia status, e.g. a control patient. In another embodiment, the control is a sample from a subject not being treated for GSD-Ia. In a still further embodiment, the control is a sample from a single subject or a pool of samples from different subjects and/or samples taken from the subject(s) at different time points.

The term “level” or “level of a biomarker” as used herein, preferably means the mass, weight or concentration of a biomarker of the invention within a sample or a subject. It will be understood by the skilled artisan that in certain embodiments the sample may be subjected to, e.g., one or more of the following: substance purification, precipitation, separation, e.g. centrifugation and/or HPLC and subsequently subjected to determining the level of the biomarker, e.g. using mass spectrometric analysis. In certain embodiments, LC-MS can be used as a means for determining the level of a biomarker according to the invention.

The term “determining the level” of a biomarker as used herein can mean methods which include quantifying an amount of at least one substance in a sample from a subject, for example, in a bodily fluid from the subject (e.g., serum, plasma, urine, lymph, etc.) or in a tissue of the subject (e.g., liver, etc.).

The term “reference level” as used herein can refer to levels (e.g., of a biomarker) in a subject prior to administration of an mRNA therapy of the invention (e.g., in a person suffering from GSD-Ia) or in a normal or healthy subject.

As used herein, the term “normal subject” or “healthy subject” refers to a subject not suffering from symptoms associated with GSD-Ia. Moreover, a subject will be considered to be normal (or healthy) if it has no mutation of the functional portions or domains of the G6PC gene and/or no mutation of the G6PC gene resulting in a reduction of or deficiency of the enzyme G6PC or the activity thereof, resulting in symptoms associated with GSD-Ia. Said mutations will be detected if a sample from the subject is subjected to a genetic testing for such G6PC mutations. In certain embodiments of the present invention, a sample from a healthy subject is used as a control sample, or the known or standardized value for the level of biomarker from samples of healthy or normal subjects is used as a control.

Since G6PC hydrolyzes G6P into glucose and phosphate during gluconeogenesis and glycogenolysis, low levels of glucose and/or phosphate, and high levels of glycogen and/or G6P, during fasting conditions is indicative that a subject has GSD-Ia or should be treated for GSD-Ia. Thus, glucose, phosphate, glycogen, and G6P can be used as biomarkers for GSD-Ia.

In some embodiments, comparing the level of the biomarker in a sample from a subject in need of treatment for GSD-Ia or in a subject being treated for GSD-Ia to a control level of the biomarker comprises comparing the level of the biomarker in the sample from the subject (in need of treatment or being treated for GSD-Ia) to a baseline or reference level, wherein if a level of the biomarker in the sample from the subject (in need of treatment or being treated for GSD-Ia) is elevated, increased or higher compared to the baseline or reference level, this is indicative that the subject is suffering from GSD-Ia and/or is in need of treatment; and/or wherein if a level of the biomarker in the sample from the subject (in need of treatment or being treated for GSD-Ia) is decreased or lower or the same compared to the baseline level this is indicative that the subject is not suffering from, is successfully being treated for GSD-Ia, or is not in need of treatment for GSD-Ia.

In some embodiments, a subject with GSD-Ia, at risk of developing GSD-Ia, or in need of treatment for GSD-Ia, has a larger liver compared to the size of a liver in a normal or healthy subject, i.e., a subject without GSD-Ia. In some embodiments, a larger liver in a subject indicates that the subject has GSD-Ia, is at risk of having GSD-Ia, or should be treated for GSD-Ia. In some embodiments, a subject having a liver that is 5%, 10%, 15%, 20%, 25%, 30%, 35%, 40%, or 50% larger than the liver in a normal or healthy subject, or to a reference weight for a normal liver, indicates that the subject has GSD-Ia or should be treated for GSD-Ia.

In some embodiments, the biomarker or biomarkers are measured in a sample taken from a subject in need of treatment for GSD-Ia or in a subject being treated for GSD-Ia that has been fasting (e.g., a subject that has not eaten for some time, e.g., for 1, 2, 3, 4, 5, 6, 7, 8, 9, 10, 11, or 12 or more hours). In some embodiments, the control sample is taken from a healthy or normal subject that has also been fasting, or the reference level of the biomarker or biomarkers are the levels in a healthy or normal subject that was fasting.

### Forms of Administration

The polynucleotides, pharmaceutical compositions and formulations of the invention described above can be administered by any route that results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal ax is), intrathoracic (within the thorax), intratubular (within the tubules of an organ), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration that is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions can be administered in a way that allows them cross the blood-brain barrier, vascular barrier, or other epithelial barrier. In some embodiments, a formulation for a route of administration can include at least one inactive ingredient.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

CONSTRUCT SEQUENCES
By “G5” is meant that all uracils (U) in the mRNAare replaced by N1-methylpseudouracils.

| | | ORF Sequence | ORF Sequence | 5′UTR | 3′UTR | Construct |
|---|---|---|---|---|---|---|
| | mRNA Name | (Amino Acid) | (Nucleotide) | Sequence | Sequence | Sequence |
| SEQ | | 1 | 2 | 55 | 114 | 5 |
| ID NO: | | | | | | |
| | G6PCS298C_G | MEEGMNVLHDFGIQSTH | AUGGAGGAGGGCAU | GGAAAU | UGAUAA | SEQ ID |
| | start_3XmiR142 | YLQVNYQDSQDWFILVS | GAACGUGCUUCACG | AAGAGA | UAGUCC | NO: 5 |
| | G5 | VIADLRNAFYVLFPIWF | ACUUCGGCAUCCAG | GAAAAG | AUAAAG | consists |
| | Cap: C1 | HLQEAVGIKLLWVAVIG | UCAACCCAUUACCU | AAGAGU | UAGGAA | from 5′ to |
| | Poly Atail: 100 nt | DWLNLVFKWILFGQRPY | ACAGGUGAACUAUC | AAGAAG | ACACUA | 3′ end: 5′ |
| | | WWVLDTDYYSNTSVPLI | AGGACUCCCAGGAU | AAAUAU | CAGCUG | UTR of |
| | | KQFPVTCETGPGSPSGH | UGGUUCAUCCUCGU | AAGAGC | GAGCCU | SEQ ID |
| | | AMGTAGVYYVMVTSTLS | GAGCGUGAUCGCCG | CACC | CGGUGG | NO: 55, |
| | | IFQGKIKPTYRFRCLNV | AUCUAAGAAACGCA | | CCUAGC | ORF |
| | | ILWLGFWAVQLNVCLSR | UUCUACGUGUUGUU | | UUCUUG | Sequence |
| | | IYLAAHFPHQVVAGVLS | CCCAAUCUGGUUCC | | CCCCUU | of SEQ ID |
| | | GIAVAETFSHIHSIYNA | ACCUGCAGGAAGCU | | GGGCCU | NO: 2, and |
| | | SLKKYFLITFFLFSFAI | GUGGGCAUCAAGCU | | CCAUAA | 3′UTR of |
| | | GFYLLLKGLGVDLLWTL | CCUUUGGGUGGCCG | | AGUAGG | SEQ ID |
| | | EKAQRWCEQPEWVHIDT | UGAUUGGCGAUUGG | | AAACAC | NO: 114 |
| | | TPFASLLKNLGTLFGLG | CUGAACCUGGUGUU | | UACAUC | |
| | | LALNSSMYRESCKGKLS | CAAGUGGAUUCUCU | | CCCCCA | |
| | | KWLPFRLSCIVASLVLL | UCGGUCAGCGGCCU | | GCCCCU | |
| | | HVFDSLKPPSQVELVFY | UACUGGUGGGUGUU | | CCUCCC | |
| | | VLSFCKSAVVPLASVSV | GGAUACCGACUAUU | | CUUCCU | |
| | | IPYCLAQVLGQPHKKSL | ACAGUAACACCUCU | | GCACCC | |
| | | | GUUCCGCUGAUCAA | | GUACCC | |
| | | | GCAGUUCCCUGUCA | | CCUCCA | |
| | | | CCUGCGAAACCGGA | | UAAAGU | |
| | | | CCUGGCUCCCCUUC | | AGGAAA | |
| | | | CGGCCACGCCAUGG | | CACUAC | |
| | | | GCACCGCCGGCGUG | | AGUGGU | |
| | | | UACUACGUGAUGGU | | CUUUGA | |
| | | | GACAUCCACCCUGA | | AUAAAG | |
| | | | GCAUCUUCCAGGGC | | UCUGAG | |
| | | | AAGAUCAAGCCUAC | | UGGGCG | |
| | | | CUACAGAUUCAGGU | | GC | |
| | | | GCCUGAACGUUAUC | | | |
| | | | UUGUGGCUGGGAUU | | | |
| | | | CUGGGCAGUGCAGC | | | |
| | | | UCAACGUGUGCCUG | | | |
| | | | AGCAGGAUCUAUCU | | | |
| | | | CGCAGCCCACUUCC | | | |
| | | | CACAUCAGGUCGUG | | | |
| | | | GCCGGCGUACUGAG | | | |
| | | | CGGAAUCGCCGUGG | | | |
| | | | CUGAAACCUUCAGC | | | |
| | | | CAUAUCCACAGCAU | | | |
| | | | CUACAAUGCCUCCC | | | |
| | | | UGAAGAAGUACUUC | | | |
| | | | CUUAUUACAUUCUU | | | |
| | | | CCUGUUCUCUUUCG | | | |
| | | | CCAUCGGCUUCUAU | | | |
| | | | CUGCUGCUAAAGGG | | | |
| | | | CCUGGGCGUCGACC | | | |
| | | | UCCUGUGGACCCUG | | | |
| | | | GAGAAGGCCCAGAG | | | |
| | | | AUGGUGCGAACAAC | | | |
| | | | CUGAGUGGGUGCAC | | | |
| | | | AUCGACACCACUCC | | | |
| | | | GUUCGCUAGCCUGC | | | |
| | | | UCAAGAACCUGGGC | | | |
| | | | ACUCUUUUCGGCCU | | | |
| | | | GGGCCUGGCCCUCA | | | |
| | | | AUAGCAGCAUGUAC | | | |
| | | | CGGGAGAGCUGUAA | | | |
| | | | GGGAAAGCUGUCCA | | | |
| | | | AGUGGCUCCCUUUC | | | |
| | | | AGACUUAGCUGCAU | | | |
| | | | CGUGGCGUCUCUCG | | | |
| | | | UGCUGCUGCACGUG | | | |
| | | | UUCGACAGCCUGAA | | | |
| | | | GCCUCCUUCGCAGG | | | |
| | | | UGGAGCUGGUGUUC | | | |
| | | | UACGUGCUGAGCUU | | | |
| | | | CUGCAAGAGCGCGG | | | |
| | | | UGGUGCCACUGGCA | | | |
| | | | AGUGUCAGCGUGAU | | | |
| | | | CCCUUAUUGCCUGG | | | |
| | | | CCCAGGUGCUGGGC | | | |
| | | | CAGCCUCAUAAGAA | | | |
| | | | GUCCCUG | | | |

### EXAMPLES

### Example 1: Chimeric Polynucleotide Synthesis

A. Triphosphate Route

Two regions or parts of a chimeric polynucleotide can be joined or ligated using triphosphate chemistry. According to this method, a first region or part of 100 nucleotides or less can be chemically synthesized with a 5′ monophosphate and terminal 3′desOH or blocked OH. If the region is longer than 80 nucleotides, it can be synthesized as two strands for ligation.

If the first region or part is synthesized as a non-positionally modified region or part using in vitro transcription (IVT), conversion the 5′monophosphate with subsequent capping of the 3′ terminus can follow. Monophosphate protecting groups can be selected from any of those known in the art.

The second region or part of the chimeric polynucleotide can be synthesized using either chemical synthesis or IVT methods. IVT methods can include an RNA polymerase that can utilize a primer with a modified cap. Alternatively, a cap of up to 80 nucleotides can be chemically synthesized and coupled to the IVT region or part.

It is noted that for ligation methods, ligation with DNA T₄ ligase, followed by treatment with DNAse should readily avoid concatenation.

The entire chimeric polynucleotide need not be manufactured with a phosphate-sugar backbone. If one of the regions or parts encodes a polypeptide, then such region or part can comprise a phosphate-sugar backbone.

Ligation can then be performed using any known click chemistry, orthoclick chemistry, solulink, or other bioconjugate chemistries known to those in the art.

B. Synthetic Route

The chimeric polynucleotide can be made using a series of starting segments. Such segments include:

(a) Capped and protected 5′ segment comprising a normal 3′OH (SEG. 1)

(b) 5′ triphosphate segment which can include the coding region of a polypeptide and comprising a normal 3′OH (SEG. 2)

(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) can be treated with cordycepin and then with pyrophosphatase to create the 5′monophosphate.

Segment 2 (SEG. 2) can then be ligated to SEG. 3 using RNA ligase. The ligated polynucleotide can then be purified and treated with pyrophosphatase to cleave the diphosphate. The treated SEG.2-SEG. 3 construct is then purified and SEG. 1 is ligated to the 5′ terminus. A further purification step of the chimeric polynucleotide can be performed.

Where the chimeric polynucleotide encodes a polypeptide, the ligated or joined segments can be represented as: 5′ UTR (SEG. 1), open reading frame or ORF (SEG. 2) and 3′ UTR+PolyA (SEG. 3).

The yields of each step can be as much as 90-95%.

### Example 2: PCR for cDNA Production

PCR procedures for the preparation of cDNA can be performed using 2×KAPA HIFI™ HotStart ReadyMix by Kapa Biosystems (Woburn, Mass.). This system includes 2×KAPA ReadyMix12.5 μl; Forward Primer (10 μM) 0.75 μl; Reverse Primer (10 μM) 0.75 μl; Template cDNA-100 ng; and dH₂0 diluted to 25.0 μl. The PCR reaction conditions can be: at 95° C. for 5 min. and 25 cycles of 98° C. for 20 sec, then 58° C. for 15 sec, then 72° C. for 45 sec, then 72° C. for 5 min. then 4° C. to termination.

The reverse primer of the instant invention can incorporate a poly-T₁₂₀ for a poly-A₁₂₀ in the mRNA. Other reverse primers with longer or shorter poly(T) tracts can be used to adjust the length of the poly(A) tail in the polynucleotide mRNA.

The reaction can be cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA can be quantified using the NANODROP™ and analyzed by agarose gel electrophoresis to confirm the cDNA is the expected size. The cDNA can then be submitted for sequencing analysis before proceeding to the in vitro transcription reaction.

### Example 3: In vitro Transcription (IVT)

The in vitro transcription reactions can generate polynucleotides containing uniformly modified polynucleotides. Such uniformly modified polynucleotides can comprise a region or part of the polynucleotides of the invention. The input nucleotide triphosphate (NTP) mix can be made using natural and un-natural NTPs.

A typical in vitro transcription reaction can include the following:
Template cDNA—1.0
10× transcription buffer (400 mM Tris-HCl pH 8.0, 190 mM MgCl₂, 50 mM DTT, 10 mM Spermidine)—2.0 μl
Custom NTPs (25 mM each)—7.2 μl
RNase Inhibitor—20 U
T7 RNA polymerase—3000 U
dH₂0—Up to 20.0 μl. and
Incubation at 37° C. for 3 hr-5 hours.

The crude IVT mix can be stored at 4° C. overnight for cleanup the next day. 1 U of RNase-free DNase can then be used to digest the original template. After 15 minutes of incubation at 37° C., the mRNA can be purified using Ambion's MEGACLEAR™ Kit (Austin, Tex.) following the manufacturer's instructions. This kit can purify up to 500 of RNA. Following the cleanup, the RNA can be quantified using the NanoDrop and analyzed by agarose gel electrophoresis to confirm the RNA is the proper size and that no degradation of the RNA has occurred.

### Example 4: Enzymatic Capping

Capping of a polynucleotide can be performed with a mixture includes: IVT RNA 60 μg-180 μg and dH₂0 up to 72 μl. The mixture can be incubated at 65° C. for 5 minutes to denature RNA, and then can be transferred immediately to ice.

The protocol can then involve the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂0 (Up to 28 μl); and incubation at 37° C. for 30 minutes for 60 μg RNA or up to 2 hours for 180 μg of RNA.

The polynucleotide can then be purified using Ambion's MEGACLEAR™ Kit (Austin, Tex.) following the manufacturer's instructions. Following the cleanup, the RNA can be quantified using the NANODROP™ (ThermoFisher, Waltham, Mass.) and analyzed by agarose gel electrophoresis to confirm the RNA is the proper size and that no degradation of the RNA has occurred. The RNA product can also be sequenced by running a reverse-transcription-PCR to generate the cDNA for sequencing.

### Example 5: PolyA Tailing Reaction

Without a poly-T in the cDNA, a poly-A tailing reaction must be performed before cleaning the final product. This can be done by mixing Capped IVT RNA (100 μl); RNase Inhibitor (20 U); 10× Tailing Buffer (0.5 M Tris-HCl (pH 8.0), 2.5 M NaCl, 100 mM MgCl₂) (12.0 μl); 20 mM ATP (6.0 μl); Poly-A Polymerase (20 U); dH₂0 up to 123.5 μl and incubating at 37° C. for 30 min. If the poly-A tail is already in the transcript, then the tailing reaction can be skipped and proceed directly to cleanup with Ambion's MEGACLEAR™ kit (Austin, Tex.) (up to 500 μg). Poly-A Polymerase is, in some cases, a recombinant enzyme expressed in yeast.

It should be understood that the processivity or integrity of the polyA tailing reaction does not always result in an exact size polyA tail. Hence polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 6: Natural 5′ Caps and 5′ Cap Analogues

5′-capping of polynucleotides can be completed concomitantly during the in vitro-transcription reaction using the following chemical RNA cap analogs to generate the 5′-guanosine cap structure according to manufacturer protocols: 3′-O-Me-m7G(5)ppp(5′) G [the ARCA cap]; G(5)ppp(5′)A; G(5′)ppp(5′)G; m7G(5′)ppp(5′)A; m7G(5′)ppp(5′)G (New England BioLabs, Ipswich, Mass.). 5′-capping of modified RNA can be completed post-transcriptionally using a Vaccinia Virus Capping Enzyme to generate the “Cap 0” structure: m7G(5′)ppp(5′)G (New England BioLabs, Ipswich, Mass.). Cap 1 structure can be generated using both Vaccinia Virus Capping Enzyme and a 2′-O methyl-transferase to generate: m7G(5′)ppp(5′)G-2′-O-methyl. Cap 2 structure can be generated from the Cap 1 structure followed by the 2′-O-methylation of the 5′-antepenultimate nucleotide using a 2′-O methyl-transferase. Cap 3 structure can be generated from the Cap 2 structure followed by the 2′-O-methylation of the 5′-preantepenultimate nucleotide using a 2′-O methyl-transferase. Enzymes can be derived from a recombinant source.

When transfected into mammalian cells, the modified mRNAs can have a stability of between 12-18 hours or more than 18 hours, e.g., 24, 36, 48, 60, 72 or greater than 72 hours.

### Example 7: Capping Assays

A. Protein Expression Assay

Polynucleotides encoding a polypeptide, containing any of the caps taught herein, can be transfected into cells at equal concentrations. After 6, 12, 24 and 36 hours post-transfection, the amount of protein secreted into the culture medium can be assayed by ELISA. Synthetic polynucleotides that secrete higher levels of protein into the medium would correspond to a synthetic polynucleotide with a higher translationally-competent Cap structure.

B. Purity Analysis Synthesis

Polynucleotides encoding a polypeptide, containing any of the caps taught herein, can be compared for purity using denaturing Agarose-Urea gel electrophoresis or HPLC analysis. Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

C. Cytokine Analysis

Polynucleotides encoding a polypeptide, containing any of the caps taught herein, can be transfected into cells at multiple concentrations. After 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to polynucleotides containing an immune-activating cap structure.

D. Capping Reaction Efficiency

Polynucleotides encoding a polypeptide, containing any of the caps taught herein, can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 8: Agarose Gel Electrophoresis of Modified RNA or RT PCR Products

Individual polynucleotides (200-400 ng in a 20 μl volume) or reverse transcribed PCR products (200-400 ng) can be loaded into a well on a non-denaturing 1.2% Agarose E-Gel (Invitrogen, Carlsbad, Calif.) and run for 12-15 minutes according to the manufacturer protocol.

### Example 9: Nanodrop Modified RNA Quantification and UV Spectral Data

Modified polynucleotides in TE buffer (1 μl) can be used for Nanodrop UV absorbance readings to quantitate the yield of each polynucleotide from a chemical synthesis or in vitro transcription reaction.

### Example 10: Method of Screening for Protein Expression

A. Electrospray Ionization

A biological sample that can contain proteins encoded by a polynucleotide administered to the subject can be prepared and analyzed according to the manufacturer protocol for electrospray ionization (ESI) using 1, 2, 3 or 4 mass analyzers. A biologic sample can also be analyzed using a tandem ESI mass spectrometry system.

Patterns of protein fragments, or whole proteins, can be compared to known controls for a given protein and identity can be determined by comparison.

B. Matrix-Assisted Laser Desorption/Ionization

A biological sample that can contain proteins encoded by one or more polynucleotides administered to the subject can be prepared and analyzed according to the manufacturer protocol for matrix-assisted laser desorption/ionization (MALDI).

Patterns of protein fragments, or whole proteins, can be compared to known controls for a given protein and identity can be determined by comparison.

C. Liquid Chromatography-Mass spectrometry-Mass spectrometry

A biological sample, which can contain proteins encoded by one or more polynucleotides, can be treated with a trypsin enzyme to digest the proteins contained within. The resulting peptides can be analyzed by liquid chromatography-mass spectrometry-mass spectrometry (LC/MS/MS). The peptides can be fragmented in the mass spectrometer to yield diagnostic patterns that can be matched to protein sequence databases via computer algorithms. The digested sample can be diluted to achieve 1 ng or less starting material for a given protein. Biological samples containing a simple buffer background (e.g., water or volatile salts) are amenable to direct in-solution digest; more complex backgrounds (e.g., detergent, non-volatile salts, glycerol) require an additional clean-up step to facilitate the sample analysis.

Patterns of protein fragments, or whole proteins, can be compared to known controls for a given protein and identity can be determined by comparison.

### Example 11: Synthesis of mRNA Encoding G6PC

Sequence optimized polynucleotides encoding G6PC polypeptides are synthesized and characterized as described in Examples 1 to 10.

An mRNA encoding human G6PC S298C (SEQ ID NO:1) can be constructed, e.g., by using the ORF sequence provided in SEQ ID NO:2. The mRNA sequence includes both 5′ and 3′ UTR regions flanking the ORF sequence. In an exemplary construct (SEQ ID NO:5), the 5′ UTR and 3′ UTR sequences are SEQ ID NO:55 and SEQ ID NO:114, respectively.

5′UTR:
(SEQ ID NO: 55)
GGAAAUAAGAGAGAAAAGAAGAGUAAGAAGAAAUAUAAGAGCCACC
3′UTR:
UGAUAAUAGUCCAUAAAGUAGGAAACACUACAGCUGGAGCCUCGGU
GGCCUAGCUUCUUGCCCCUUGGGCCUCCAUAAAGUAGGAAACACUA
CAUCCCCCCAGCCCCUCCUCCCCUUCCUGCACCCGUACCCCCUCCA
UAAAGUAGGAAACACUACAGUGGUCUUUGAAUAAAGUCUGAGUGGG
CGGC: 114)

The G6PC mRNA sequence is prepared as modified mRNA. Specifically, during in vitro transcription, modified mRNA can be generated using N₁-methylpseudouridine-5′-triphosphate to ensure that the mRNAs contain 100% N₁-methylpseudouridine instead of uridine. Further, G6PC-mRNA can be synthesized with a primer that introduces a polyA-tail, and a Cap 1 structure is generated on both mRNAs using Vaccinia Virus Capping Enzyme and a 2′-O methyl-transferase to generate: m7G(5)ppp(5′)G-2′-O-methyl.

### Example 12: Production of Nanoparticle Compositions

A. Production of Nanoparticle Compositions

Nanoparticles can be made with mixing processes such as microfluidics and T-junction mixing of two fluid streams, one of which contains the polynucleotide and the other has the lipid components.

Lipid compositions are prepared by combining an ionizable amino lipid disclosed herein, e.g., Compound A, a phospholipid (such as DOPE or DSPC, obtainable from Avanti Polar Lipids, Alabaster, Ala.), a PEG lipid (such as Compound I), and a structural lipid (such as cholesterol, obtainable from Sigma-Aldrich, Taufkirchen, Germany, or a corticosteroid (such as prednisolone, dexamethasone, prednisone, and hydrocortisone), or a combination thereof) at concentrations of about 50 mM in ethanol. Solutions should be refrigerated for storage at, for example, −20° C. Lipids are combined to yield desired molar ratios and diluted with water and ethanol to a final lipid concentration of between about 5.5 mM and about 25 mM.

Nanoparticle compositions including a polynucleotide and a lipid composition are prepared by combining the lipid solution with a solution including the a polynucleotide at lipid composition to polynucleotide wt:wt ratios between about 5:1 and about 50:1. The lipid solution is rapidly injected using a NanoAssemblr microfluidic based system at flow rates between about 10 ml/min and about 18 ml/min into the polynucleotide solution to produce a suspension with a water to ethanol ratio between about 1:1 and about 4:1.

For nanoparticle compositions including an RNA, solutions of the RNA at concentrations of 0.1 mg/ml in deionized water are diluted in 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kD. The first dialysis is carried out at room temperature for 3 hours. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, can be used to achieve the same nano-precipitation.

B. Characterization of nanoparticle compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of polynucleotide in the nanoparticle composition can be calculated based on the extinction coefficient of the polynucleotide used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

Exemplary formulations of the nanoparticle compositions are presented in the Table 4 below. The term “Compound” refers to an ionizable lipid (e.g., Compound A). “Phospholipid” can be, e.g., DSPC or DOPE. “PEG-lipid” can be, e.g., PEG-DMG or Compound I.

TABLE 4
Exemplary Formulations of Nanoparticles

| Composition (mol %) | Components |
|---|---|
| 40:20:38.5:1.5 | Compound:Phospholipid:Chol:PEG-lipid |
| 45:15:38.5:1.5 | Compound:Phospholipid:Chol:PEG-lipid |
| 50:10:38.5:1.5 | Compound:Phospholipid:Chol:PEG-lipid |
| 55:5:38.5:1.5 | Compound:Phospholipid:Chol:PEG-lipid |
| 60:5:33.5:1.5 | Compound:Phospholipid:Chol:PEG-lipid |
| 45:20:33.5:1.5 | Compound:Phospholipid:Chol:PEG-lipid |
| 50:20:28.5:1.5 | Compound:Phospholipid:Chol:PEG-lipid |
| 55:20:23.5:1.5 | Compound:Phospholipid:Chol:PEG-lipid |
| 60:20:18.5:1.5 | Compound:Phospholipid:Chol:PEG-lipid |
| 40:15:43.5:1.5 | Compound:Phospholipid:Chol:PEG-lipid |
| 50:15:33.5:1.5 | Compound:Phospholipid:Chol:PEG-lipid |
| 55:15:28.5:1.5 | Compound:Phospholipid:Chol:PEG-lipid |
| 60:15:23.5:1.5 | Compound:Phospholipid:Chol:PEG-lipid |
| 40:10:48.5:1.5 | Compound:Phospholipid:Chol:PEG-lipid |
| 45:10:43.5:1.5 | Compound:Phospholipid:Chol:PEG-lipid |
| 55:10:33.5:1.5 | Compound:Phospholipid:Chol:PEG-lipid |
| 60:10:28.5:1.5 | Compound:Phospholipid:Chol:PEG-lipid |
| 40:5:53.5:1.5 | Compound:Phospholipid:Chol:PEG-lipid |
| 45:5:48.5:1.5 | Compound:Phospholipid:Chol:PEG-lipid |
| 50:5:43.5:1.5 | Compound:Phospholipid:Chol:PEG-lipid |
| 40:20:40:0 | Compound:Phospholipid:Chol:PEG-lipid |
| 45:20:35:0 | Compound:Phospholipid:Chol:PEG-lipid |
| 50:20:30:0 | Compound:Phospholipid:Chol:PEG-lipid |
| 55:20:25:0 | Compound:Phospholipid:Chol:PEG-lipid |
| 60:20:20:0 | Compound:Phospholipid:Chol:PEG-lipid |
| 40:15:45:0 | Compound:Phospholipid:Chol:PEG-lipid |
| 45:15:40:0 | Compound:Phospholipid:Chol:PEG-lipid |
| 50:15:35:0 | Compound:Phospholipid:Chol:PEG-lipid |
| 55:15:30:0 | Compound:Phospholipid:Chol:PEG-lipid |
| 60:15:25:0 | Compound:Phospholipid:Chol:PEG-lipid |
| 40:10:50:0 | Compound:Phospholipid:Chol:PEG-lipid |
| 45:10:45:0 | Compound:Phospholipid:Chol:PEG-lipid |
| 50:10:40:0 | Compound:Phospholipid:Chol:PEG-lipid |
| 55:10:35:0 | Compound:Phospholipid:Chol:PEG-lipid |
| 60:10:30:0 | Compound:Phospholipid:Chol:PEG-lipid |

### Example 13: Single Dosing of mRNA-Encoded G6PC in a Liver-Specific Knockout Mouse Model

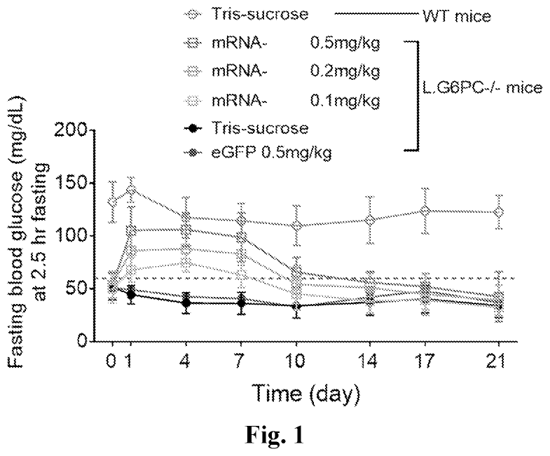

The liver-specific G6PC knock-out mouse (L-G6PC(−/−)) is a genetic mouse model of GSD-Ia (Mutel et al., J. Hepatol., 2011, 54(3):529-37, herein incorporated by reference in its entirety). To test the efficacy and duration of action of a 1-methyl-pseudouridine modified mRNA encoding human G6PC S298C (SEQ ID NO:1) in vivo, a single 0.1 mg/kg, 0.2 mg/kg, or 0.5 mg/kg dose of mRNA (construct of SEQ ID NO:5) was intravenously administered to L-G6PC(−/−) mice via tail vein injection. The mRNA was formulated in lipid nanoparticles (containing Compound A and Compound I) for delivery into the mice. As controls, Tris-sucrose or an mRNA encoding eGFP (at 0.5 mg/kg) were injected into L-G6PC(−/−) mice and a Tris-sucrose control was injected into wild-type mice. Blood glucose levels were monitored at 2.5 hours fasting one day prior to mRNA injection (day 0), the day of injection (day 1), and each of days 4, 7, 10, 14, 17, and 21. Mice administered the mRNA formulated in lipid nanoparticles exhibited blood glucose levels significantly higher than those of mice injected with the eGFP or Tris-sucrose controls for up to 10 days (FIG. 1).

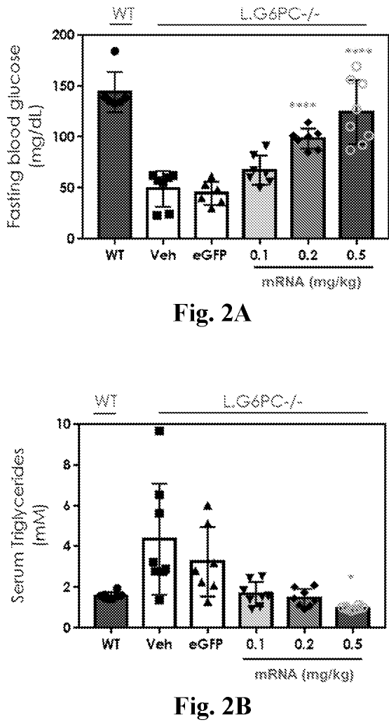

Four weeks after the first administration, the same cohort of L-G6PC(−/−) mice were injected with an additional dose of mRNA (construct of SEQ ID NO:5) formulated in lipid nanoparticles (containing Compound A and Compound I) at 0.1 mg/kg, 0.2 mg/kg, or 0.5 mg/kg intravenously via tail vein injection. As controls, Tris-sucrose or an mRNA encoding eGFP were injected into L-G6PC(−/−) mice and a Tris-sucrose control was injected into wild-type mice. Fasting was immediately initiated after administration of the mRNA. Blood glucose levels and serum triglyceride levels were monitored after a 6 hour fast (blood glucose) or 24 hour fast (serum triglyceride) immediately following injection of the lipid nanoparticles. Mice administered the mRNA formulated in lipid nanoparticles exhibited fasting blood glucose levels significantly higher than those of mice injected with the controls at 6 hours after injection (FIG. 2A). Mice administered the mRNA formulated in lipid nanoparticles exhibited serum triglyceride levels significantly lower than those of mice injected with the controls at 24 hours after injection (FIG. 2B).

A single 0.1 mg/kg, 0.2 mg/kg, or 0.5 mg/kg dose of mRNA (construct of SEQ ID NO:5) formulated in lipid nanoparticles (containing Compound A and Compound I) was intravenously administered to L-G6PC(−/−) mice via tail vein injection. As controls, Tris-sucrose or mRNA encoding eGFP were injected into L-G6PC(−/−) mice and a Tris-sucrose control was injected into wild-type mice. At 24 hours post-injection, liver samples were collected and liver weight, G6P protein levels, glycogen levels, triglyceride levels, G6Pase activity, and human G6Pase protein expression levels were measured in the liver samples.

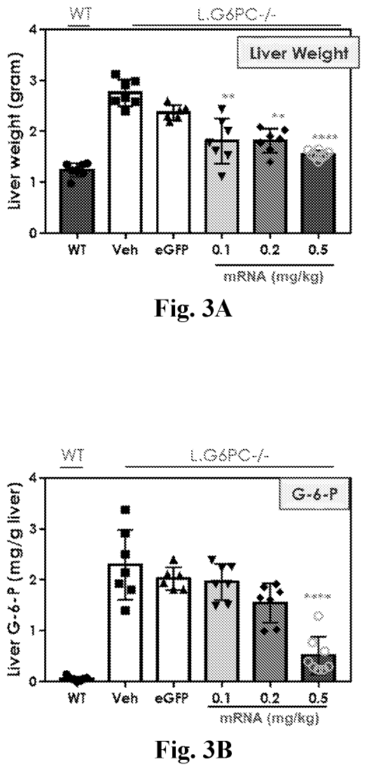

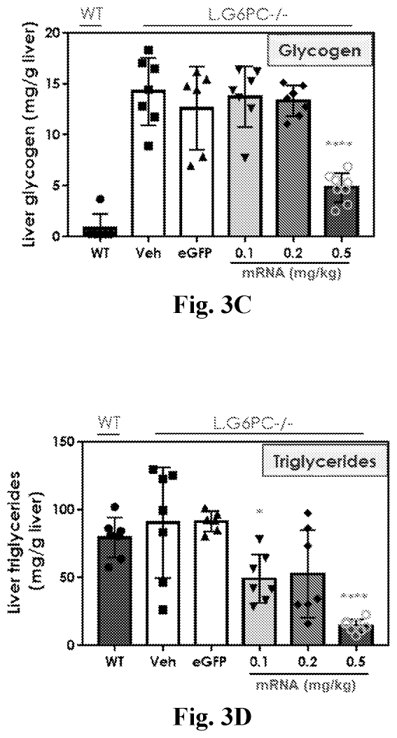

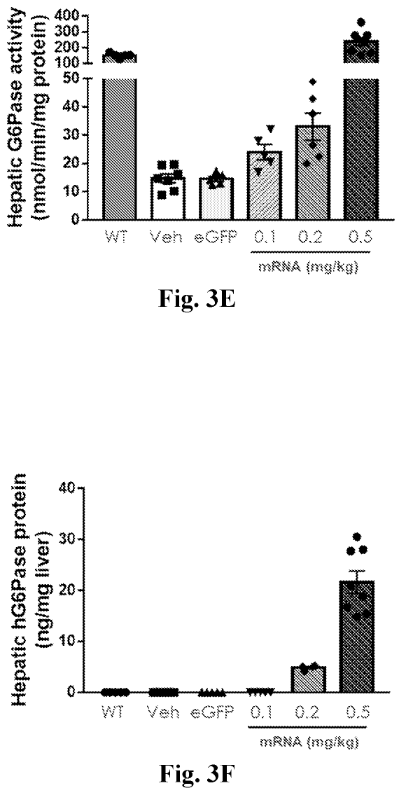

As expected, control L-G6PC(−/−) mice injected with Tris-sucrose or mRNA encoding eGFP had substantially larger livers compared to wild-type control mice (FIG. 3A). Injection of the mRNA formulated in lipid nanoparticles resulted in a significant decrease in liver weight (FIG. 3A).

As expected, the hepatic G6P content from control L-G6PC(−/−) mice injected with Tris-sucrose or mRNA encoding eGFP was significantly elevated in comparison with wild-type mice (FIG. 3B). Injection of the mRNA formulated in lipid nanoparticles led to a significant decrease in hepatic G6P content (FIG. 3B).

As expected, the hepatic glycogen content from control L-G6PC(−/−) mice injected with Tris-sucrose or mRNA encoding eGFP was significantly elevated in comparison with wild-type mice (FIG. 3C). Injection of the mRNA formulated in lipid nanoparticles led to a significant decrease in hepatic glycogen content (FIG. 3C).

As expected, the hepatic triglyceride level from control L-G6PC(−/−) mice injected with Tris-sucrose or mRNA encoding eGFP was significantly elevated in comparison with wild-type mice (FIG. 3D). Injection of the mRNA formulated in lipid nanoparticles led to a significant decrease in hepatic serum triglyceride levels (FIG. 3D).

As expected, the hepatic G6Pase activity level in control L-G6PC(−/−) mice injected with Tris-sucrose or mRNA encoding eGFP was significantly reduced in comparison with wild-type mice (FIG. 3E). Injection of the mRNA formulated in lipid nanoparticles led to a dose-dependent increase in hepatic G6Pase activity (FIG. 3E).

As expected, the human G6Pase protein was not expressed in livers of wild-type mice or control L-G6PC(−/−) mice injected with Tris-sucrose or mRNA encoding eGFP (FIG. 3F). Injection of the mRNA formulated in lipid nanoparticles at 0.2 mg/kg and 0.5 mg/kg led to a significant increase in hepatic human G6Pase protein levels (FIG. 3F).

### Example 14: Repeat Dosing of mRNA-Encoded G6PC in a Liver-Specific Knockout Mouse Model

L-G6PC(−/−) mice received three 0.2 mg/kg intravenous tail vein injections, at 14 day intervals, of a 1-methyl-pseudouridine modified mRNA (construct of SEQ ID NO:5, encoding the human G6PC S298C protein of SEQ ID NO:1) formulated in lipid nanoparticles (containing Compound A and Compound I). As controls, Tris-sucrose or mRNA encoding eGFP (at 0.2 mg/kg) were injected into L-G6PC(−/−) mice and a Tris-sucrose control was injected into wild-type mice.

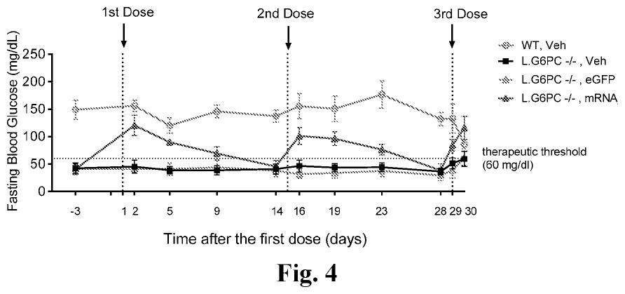

L-G6PC(−/−) mice that were administered multiple doses of 0.2 mg/kg of the mRNA formulated in lipid nanoparticles exhibited higher blood glucose levels than L-G6PC(−/−) mice injected with the controls after being fasted for 2.5 hours (FIG. 4). Injection of each of the three doses the mRNA formulated in lipid nanoparticles resulted in blood glucose levels in L-G6PC(−/−) mice that were above the therapeutic threshold of 60 mg/dL (indicated by a solid bar in FIG. 4) for at least 9 days after each injection. As expected, wild-type mice had high blood glucose levels when fasted.

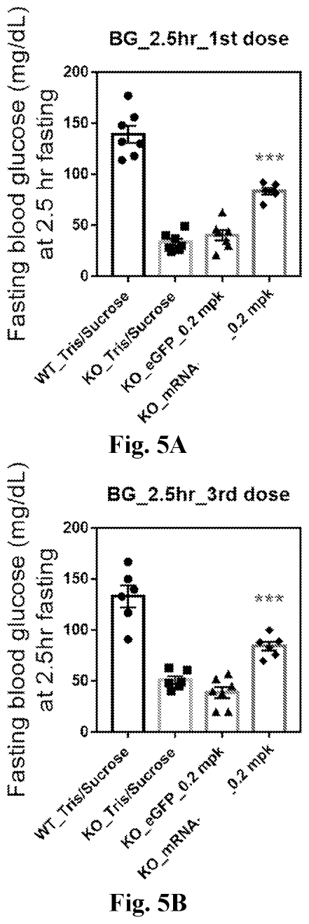

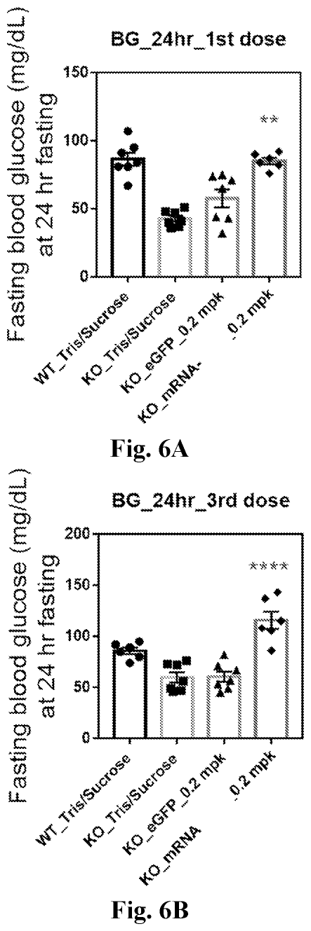

Blood glucose levels were monitored at 2.5 hours after administration of each of the first and third doses, with fasting initiated immediately after each of the administrations. L-G6PC(−/−) mice administered 0.2 mg/kg of the mRNA formulated in lipid nanoparticles exhibited after the first dose (FIG. 5A) and the third dose (FIG. 5B) blood glucose levels significantly higher than those of control L-G6PC(−/−) mice injected with Tris-sucrose or mRNA encoding eGFP. The level of improvement in fasting blood glucose was similar after the first and third dose (FIGS. 5A and 5B).

Blood glucose levels were monitored at 24 hours after administration of each of the first and third doses, with fasting initiated immediately after each of the administrations. L-G6PC(−/−) mice administered 0.2 mg/kg of the mRNA formulated in lipid nanoparticles exhibited after the first dose (FIG. 6A) and the third dose (FIG. 6B) blood glucose levels significantly higher than those of control L-G6PC(−/−) mice injected with Tris-sucrose or mRNA encoding eGFP. The level of improvement in fasting blood glucose was similar after the first and third dose (FIGS. 6A and 6B).

### Example 15: Therapeutic Impact of hG6PC mRNA-Lipid Nanoparticle in Long-Term GSD1a Pathology

To evaluate the effect of N₁-methylpseudouridine-modified mRNA encoding G6PC S298C (SEQ ID NO:1) on prevention of hepatocellular adenomas (HCA)/hepatocellular carcinomas (HCC), HCC was induced in L.G6pc−/− mice (Mutel et al. J Hepatol 54, 529-537 (2011)) by feeding them a high fat/high sucrose (HD/HS) diet using the protocol described in Gjorgjieva et al. J Hepatol 69, 1074-1087 (2018), herein incorporated by reference in its entirety. These L.G6pc−/− mice were then administered a 0.25 mg/kg dose of mRNA (construct SEQ ID NO:6) or control eGFP mRNA once every two weeks for a total of 10 doses. mRNA was formulated in lipid nanoparticles (containing PEG-DMG and a compound having the following structure:

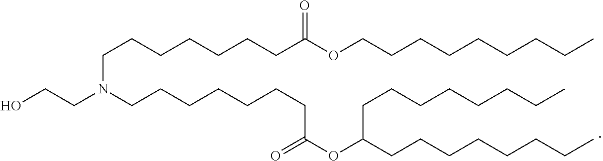 [SMILES: CCCCCCCCCOC(=O)CCCCCCCN(CCO)CCCCCCCC(=O)OC(CCCCCCCC)CCCCCCCC]

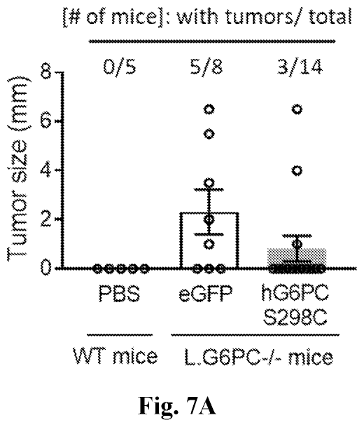

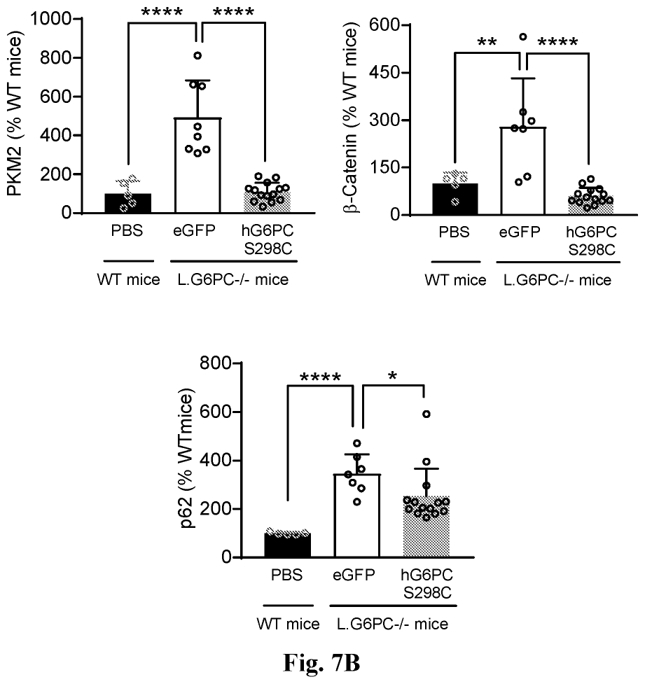

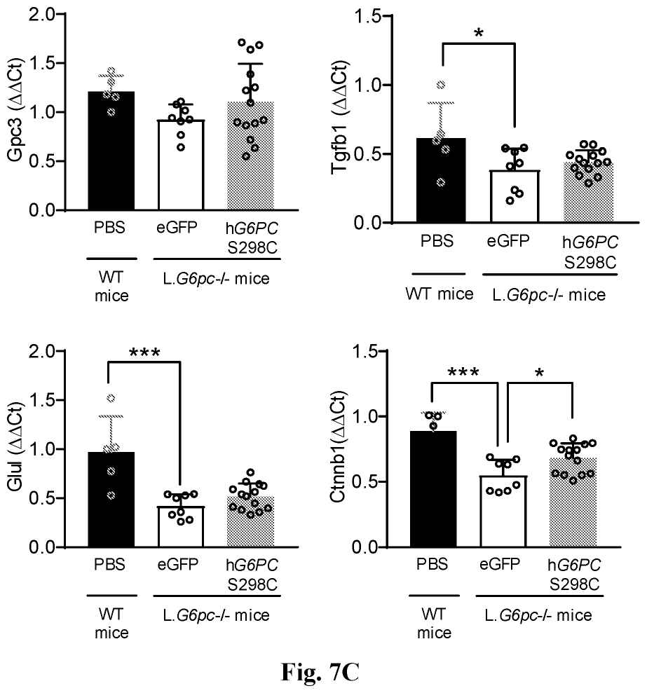

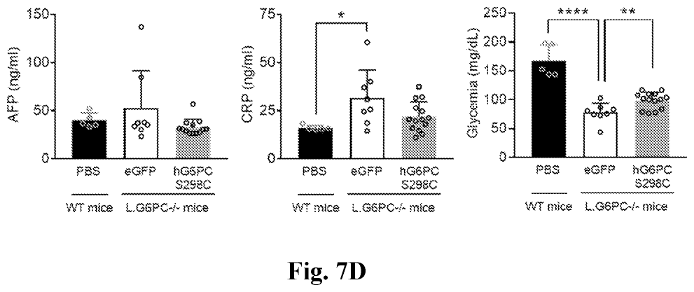

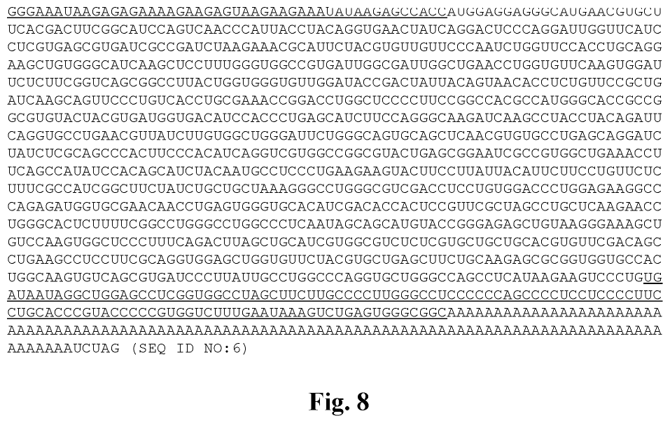

mRNA administration was performed via tail vein injection. Mice were euthanized 8 days after the last mRNA administration and livers were harvested, weighed, and photographed. Liver fragments were snap-frozen in liquid nitrogen and kept at −80° C. for further use. While no tumors were observed in WT mice livers, multiple tumors (as large as ˜7 mm) were observed in the livers of L.G6pc−/− mice treated with control, eGFP mRNA (FIG. 7A). In contrast, while not statistically significant, fewer tumors were observed in the livers of mice treated with hG6PC S298C mRNA (SEQ ID NO:6) (FIG. 7A). Indeed, most of these treated mice (11 out of 14) did not develop macroscopic tumors. These observations were further confirmed by histological analysis of the representative liver sections from each cohort. Moreover, HCA/HCC-related biomarkers (PKM2, β-catenin, and p62) (FIG. 7B) and genes associated with cellular proliferation (Gpc3, Tgfb1, Glul, and Ctnnb1) were partially reversed with hG6PC S298C mRNA treatment (FIG. 7C). In addition, serum biomarkers associated with GSD1a (glycemia) and HCA/HCC (AFP and CRP) trended towards normal levels upon treatment with hG6PC S298C mRNA (FIG. 7D).

### Example 16: Repeat Dosing of mRNA-Encoded G6PC in a Liver-Specific Knockout Mouse Model of HCC/HCA

To evaluate the efficacy of a 1-methyl-pseudouridine modified mRNA encoding human G6PC S298C (SEQ ID NO:1) in the treatment and prevention of HCC/HCCA in vivo, HCC is induced in L-G6PC(−/−) mice by feeding them a HF/HS diet using the protocol described in Gjorgjieva et al. J Hepatol 69, 1074-1087 (2018). The mice are then administered a 0.25 mg/kg dose of mRNA (construct SEQ ID NO:5) once every two weeks for a total of 10 doses. Administrations of mRNA are performed via tail vein injection. The mRNA is formulated in lipid nanoparticles (containing Compound A and Compound I) for delivery into the mice. As controls, an mRNA encoding eGFP (at 0.25 mg/kg) is injected into the HF/HS L-G6PC(−/−) mice and a phosphate buffered saline is injected into wild-type mice. Eight days after the tenth of ten doses, mice are euthanized, livers are harvested, weighed, and photographed. Liver fragments are snap-frozen in liquid nitrogen and kept at −80° C. for further use.

Livers are examined for tumors and the total number of mice bearing liver tumors is determined. Histological analysis of liver sections is also performed. Livers are also examined for HCA/HCC-related biomarkers (PKM2, β-catenin, and p62), genes associated with cellular proliferation (Gpc3, Tgfb1, Glul, and Ctnnb1), and serum biomarkers associated with GSD1a (glycemia) and HCA/HCC (AFP and CRP).

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All publications, patent applications, patents, and other references mentioned herein are incorporated by reference in their entirety. In case of conflict, the present specification, including definitions, will control.

What is claimed is:

1. A lipid nanoparticle comprising: 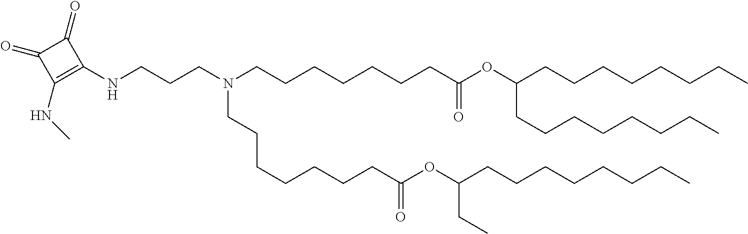 [SMILES: CCCCCCCCC(CC)OC(=O)CCCCCCCN(CCCCCCCC(=O)OC(CCCCCCCC)CCCCCCCC)CCCNc1c(NC)c(=O)c1=O]
(Compound A) or its N-oxide, or a salt or isomer thereof; and
a messenger RNA (mRNA) comprising an open reading frame (ORF) encoding the polypeptide of SEQ ID NO:1, wherein the ORF comprises the nucleic acid sequence of SEQ ID NO:2.

2. The lipid nanoparticle of claim 1, wherein the mRNA comprises a 5′ UTR comprising the nucleic acid sequence of SEQ ID NO:55.

3. The lipid nanoparticle of claim 1, wherein the mRNA comprises a 3′ UTR comprising the nucleic acid sequence of SEQ ID NO:114.

4. The lipid nanoparticle of claim 1, wherein the mRNA comprises the nucleic acid sequence of SEQ ID NO:5.

5. The lipid nanoparticle of any one of claims 1 to 4, wherein the mRNA comprises a 5′ terminal cap.

6. The lipid nanoparticle of claim 5, wherein the 5′ terminal cap comprises a guanine cap nucleotide containing an N₇ methylation and the 5′-terminal nucleotide of the mRNA contains a 2′-O-methyl.

7. The lipid nanoparticle of any one of claims 1 to 6, wherein the mRNA comprises a poly-A region.

8. The lipid nanoparticle of claim 7, wherein the poly-A region is at least about 100 nucleotides in length.

9. The lipid nanoparticle of any one of claims 1 to 8, wherein all of the uracils of the mRNA are N₁-methylpseudouracils.

10. The lipid nanoparticle of claim 1, wherein the mRNA comprises a 5′ terminal cap comprising a guanine cap nucleotide containing an N₇ methylation and the 5′-terminal nucleotide of the mRNA contains a 2′-O-methyl, wherein the mRNA comprises the nucleotide sequence of SEQ ID NO:5, wherein the mRNA comprises a poly-A region at least about 100 nucleotides in length, and wherein all of the uracils of the mRNA are N₁-methylpseudouracils.

11. The lipid nanoparticle of any one of claims 1 to 10, wherein the lipid nanoparticle comprises Compound I.

12. The lipid nanoparticle of any one of claims 1 to 11, wherein the lipid nanoparticle comprises a phospholipid and a structural lipid.

13. The lipid nanoparticle of any one of claims 1 to 10, wherein the lipid nanoparticle comprises: (a) cholesterol and Compound I; (b) cholesterol and PEG-DMG; (c) DSPC, cholesterol, and Compound I; (d) DOPE, cholesterol, and Compound I; (e) DSPC, cholesterol, and PEG-DMG; or (f) DOPE, cholesterol, and PEG-DMG.

14. A method of expressing a glucose-6-phosphatase (G6PC) polypeptide in a human subject in need thereof, comprising administering to the human subject an effective amount of the lipid nanoparticle of any one of claims 1 to 13.

15. A method of treating, preventing, or delaying the onset and/or progression of glycogen storage disease type 1a (GSD-Ia) in a human subject in need thereof, comprising administering to the human subject an effective amount of the lipid nanoparticle of any one of claims 1 to 13.

16. A method of increasing blood, plasma, and/or serum glucose levels in a human subject in need thereof, comprising administering to the human subject an effective amount of the lipid nanoparticle of any one of claims 1 to 13.

17. A method of reducing liver glycogen levels in a human subject in need thereof, comprising administering to the human subject an effective amount of the lipid nanoparticle of any one of claims 1 to 13.

18. A method of reducing liver glucose-6-phosphate (G6P) levels in a human subject in need thereof, comprising administering to the human subject an effective amount of the lipid nanoparticle of any one of claims 1 to 13.

19. A method of reducing serum and/or liver triglyceride levels in a human subject in need thereof, comprising administering to the human subject an effective amount of the lipid nanoparticle of any one of claims 1 to 13.

20. A method of increasing G6PC activity in a human subject in need thereof, comprising administering to the human subject an effective amount of the lipid nanoparticle of any one of claims 1 to 13.

21. The method of claim 20, wherein the G6PC activity is increased in the liver and/or blood of the human subject.

22. A method of treating or preventing liver adenoma in a human subject having glycogen storage disease type 1a (GSD-Ia), comprising administering to the human subject an effective amount of the lipid nanoparticle of any one of claims 1 to 13.

23. A method of treating or preventing liver carcinoma in a human subject having glycogen storage disease type 1a (GSD-Ia), comprising administering to the human subject an effective amount of the lipid nanoparticle of any one of claims 1 to 13.

24. The method of any one of claims 14 to 23, wherein the human subject has been fasting.

25. The method of claim 24, wherein the human subject has been fasting for at least 2 hours.

26. The method of any one of claims 14 to 25, comprising multiple administrations of the lipid nanoparticle to the human subject.

27. The method of claim 26, wherein the multiple administrations to the human subject are about once a week, about once every two weeks, or about once a month.

28. The method of any one of claims 14 to 27, wherein the mRNA is administered at a dose of 0.1 mg mRNA/kg of body weight of the human subject.

29. The method of any one of claims 14 to 27, wherein the mRNA is administered at a dose of 0.2 mg mRNA/kg of body weight of the human subject.

30. The method of any one of claims 14 to 27, wherein the mRNA is administered at a dose of 0.5 mg mRNA/kg of body weight of the human subject.

31. The method of any one of claims 14 to 30, wherein the lipid nanoparticle is administered intravenously.


SEQUENCE LISTING (214 sequences)

SEQ ID NO: 1 - protein, 357 aa, Artificial Sequence
  source: /note=“Description of Artificial Sequence: Synthetic polypeptide”
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLSCIVASLVLLHVFDSLKPPSQVELVFYVLSFCKSAVVPLASVSVIPYCLAQVLGQPHKKSL

SEQ ID NO: 2 - RNA, 1071 nt, Artificial Sequence
  source: /note=“Description of Artificial Sequence: Synthetic polynucleotide”
auggaggagggcaugaacgugcuucacgacuucggcauccagucaacccauuaccuacaggugaacuaucaggacucccaggauugguucauccucgugagcgugaucgccgaucuaagaaacgcauucuacguguuguucccaaucugguuccaccugcaggaagcugugggcaucaagcuccuuuggguggccgugauuggcgauuggcugaaccugguguucaaguggauucucuucggucagcggccuuacugguggguguuggauaccgacuauuacaguaacaccucuguuccgcugaucaagcaguucccugucaccugcgaaaccggaccuggcuccccuuccggccacgccaugggcaccgccggcguguacuacgugauggugacauccacccugagcaucuuccagggcaagaucaagccuaccuacagauucaggugccugaacguuaucuuguggcugggauucugggcagugcagcucaacgugugccugagcaggaucuaucucgcagcccacuucccacaucaggucguggccggcguacugagcggaaucgccguggcugaaaccuucagccauauccacagcaucuacaaugccucccugaagaaguacuuccuuauuacauucuuccuguucucuuucgccaucggcuucuaucugcugcuaaagggccugggcgucgaccuccuguggacccuggagaaggcccagagauggugcgaacaaccugagugggugcacaucgacaccacuccguucgcuagccugcucaagaaccugggcacucuuuucggccugggccuggcccucaauagcagcauguaccgggagagcuguaagggaaagcuguccaaguggcucccuuucagacuuagcugcaucguggcgucucucgugcugcugcacguguucgacagccugaagccuccuucgcagguggagcugguguucuacgugcugagcuucugcaagagcgcgguggugccacuggcaagugucagcgugaucccuuauugccuggcccaggugcugggccagccucauaagaagucccug

SEQ ID NO: 3-4 - skipped (2 entries)

SEQ ID NO: 5 - RNA, 1305 nt, Artificial Sequence
  source: /note=“Description of Artificial Sequence: Synthetic polynucleotide”
  modified_base (6)..(6): N1-methylpseudouracil
  modified_base (24)..(24): N1-methylpseudouracil
  modified_base (34)..(34): N1-methylpseudouracil
  modified_base (36)..(36): N1-methylpseudouracil
  modified_base (48)..(48): N1-methylpseudouracil
  modified_base (60)..(60): N1-methylpseudouracil
  modified_base (66)..(66): N1-methylpseudouracil
  modified_base (69)..(70): N1-methylpseudouracil
  modified_base (77)..(78): N1-methylpseudouracil
  modified_base (84)..(84): N1-methylpseudouracil
  modified_base (89)..(89): N1-methylpseudouracil
  modified_base (97)..(98): N1-methylpseudouracil
  modified_base (102)..(102): N1-methylpseudouracil
  modified_base (108)..(108): N1-methylpseudouracil
  modified_base (113)..(113): N1-methylpseudouracil
  modified_base (115)..(115): N1-methylpseudouracil
  modified_base (122)..(122): N1-methylpseudouracil
  modified_base (130)..(131): N1-methylpseudouracil
  modified_base (134)..(135): N1-methylpseudouracil
  modified_base (138)..(138): N1-methylpseudouracil
  modified_base (141)..(141): N1-methylpseudouracil
  modified_base (144)..(144): N1-methylpseudouracil
  modified_base (150)..(150): N1-methylpseudouracil
  modified_base (153)..(153): N1-methylpseudouracil
  modified_base (160)..(160): N1-methylpseudouracil
  modified_base (162)..(162): N1-methylpseudouracil
  modified_base (173)..(174): N1-methylpseudouracil
  modified_base (176)..(176): N1-methylpseudouracil
  modified_base (180)..(180): N1-methylpseudouracil
  modified_base (182)..(183): N1-methylpseudouracil
  modified_base (185)..(186): N1-methylpseudouracil
  modified_base (192)..(192): N1-methylpseudouracil
  modified_base (194)..(194): N1-methylpseudouracil
  modified_base (197)..(198): N1-methylpseudouracil
  modified_base (204)..(204): N1-methylpseudouracil
  modified_base (214)..(214): N1-methylpseudouracil
  modified_base (216)..(216): N1-methylpseudouracil
  modified_base (222)..(222): N1-methylpseudouracil
  modified_base (228)..(228): N1-methylpseudouracil
  modified_base (231)..(233): N1-methylpseudouracil
  modified_base (237)..(237): N1-methylpseudouracil
  modified_base (243)..(243): N1-methylpseudouracil
  modified_base (246)..(247): N1-methylpseudouracil
  modified_base (253)..(254): N1-methylpseudouracil
  modified_base (258)..(258): N1-methylpseudouracil
  modified_base (264)..(264): N1-methylpseudouracil
  modified_base (267)..(267): N1-methylpseudouracil
  modified_base (269)..(270): N1-methylpseudouracil
  modified_base (275)..(275): N1-methylpseudouracil
  modified_base (279)..(280): N1-methylpseudouracil
  modified_base (282)..(282): N1-methylpseudouracil
  modified_base (284)..(285): N1-methylpseudouracil
  modified_base (289)..(289): N1-methylpseudouracil
  modified_base (298)..(299): N1-methylpseudouracil
  modified_base (302)..(302): N1-methylpseudouracil
  modified_base (305)..(305): N1-methylpseudouracil
  modified_base (309)..(309): N1-methylpseudouracil
  modified_base (311)..(312): N1-methylpseudouracil
  modified_base (316)..(316): N1-methylpseudouracil
  modified_base (323)..(323): N1-methylpseudouracil
  modified_base (325)..(326): N1-methylpseudouracil
  modified_base (331)..(331): N1-methylpseudouracil
  modified_base (338)..(338): N1-methylpseudouracil
  modified_base (340)..(340): N1-methylpseudouracil
  modified_base (342)..(343): N1-methylpseudouracil
  modified_base (348)..(348): N1-methylpseudouracil
  modified_base (351)..(351): N1-methylpseudouracil
  modified_base (359)..(360): N1-methylpseudouracil
  modified_base (364)..(364): N1-methylpseudouracil
  modified_base (366)..(366): N1-methylpseudouracil
  modified_base (371)..(371): N1-methylpseudouracil
  modified_base (385)..(385): N1-methylpseudouracil
  modified_base (389)..(389): N1-methylpseudouracil
  modified_base (394)..(395): N1-methylpseudouracil
  modified_base (408)..(408): N1-methylpseudouracil
  modified_base (423)..(423): N1-methylpseudouracil
  modified_base (425)..(425): N1-methylpseudouracil
  modified_base (428)..(428): N1-methylpseudouracil
  modified_base (432)..(432): N1-methylpseudouracil
  modified_base (435)..(435): N1-methylpseudouracil
  modified_base (438)..(438): N1-methylpseudouracil
  modified_base (443)..(443): N1-methylpseudouracil
  modified_base (450)..(450): N1-methylpseudouracil
  modified_base (456)..(456): N1-methylpseudouracil
  modified_base (458)..(459): N1-methylpseudouracil
  modified_base (471)..(471): N1-methylpseudouracil
  modified_base (478)..(478): N1-methylpseudouracil
  modified_base (482)..(482): N1-methylpseudouracil
  modified_base (488)..(489): N1-methylpseudouracil
  modified_base (494)..(494): N1-methylpseudouracil
  modified_base (498)..(498): N1-methylpseudouracil
  modified_base (504)..(505): N1-methylpseudouracil
  modified_base (507)..(507): N1-methylpseudouracil
  modified_base (509)..(510): N1-methylpseudouracil
  modified_base (512)..(512): N1-methylpseudouracil
  modified_base (516)..(516): N1-methylpseudouracil
  modified_base (521)..(522): N1-methylpseudouracil
  modified_base (524)..(524): N1-methylpseudouracil
  modified_base (531)..(531): N1-methylpseudouracil
  modified_base (537)..(537): N1-methylpseudouracil
  modified_base (543)..(543): N1-methylpseudouracil
  modified_base (545)..(545): N1-methylpseudouracil
  modified_base (549)..(549): N1-methylpseudouracil
  modified_base (558)..(558): N1-methylpseudouracil
  modified_base (560)..(560): N1-methylpseudouracil
  modified_base (562)..(562): N1-methylpseudouracil
  modified_base (564)..(564): N1-methylpseudouracil
  modified_base (575)..(576): N1-methylpseudouracil
  modified_base (583)..(583): N1-methylpseudouracil
  modified_base (588)..(588): N1-methylpseudouracil
  modified_base (591)..(591): N1-methylpseudouracil
  modified_base (600)..(600): N1-methylpseudouracil
  modified_base (603)..(603): N1-methylpseudouracil
  modified_base (612)..(612): N1-methylpseudouracil
  modified_base (618)..(618): N1-methylpseudouracil
  modified_base (622)..(622): N1-methylpseudouracil
  modified_base (629)..(630): N1-methylpseudouracil
  modified_base (637)..(637): N1-methylpseudouracil
  modified_base (639)..(639): N1-methylpseudouracil
  modified_base (648)..(648): N1-methylpseudouracil
  modified_base (650)..(650): N1-methylpseudouracil
  modified_base (655)..(655): N1-methylpseudouracil
  modified_base (659)..(659): N1-methylpseudouracil
  modified_base (663)..(663): N1-methylpseudouracil
  modified_base (671)..(671): N1-methylpseudouracil
  modified_base (674)..(675): N1-methylpseudouracil
  modified_base (678)..(679): N1-methylpseudouracil
  modified_base (681)..(682): N1-methylpseudouracil
  modified_base (686)..(687): N1-methylpseudouracil
  modified_base (689)..(690): N1-methylpseudouracil
  modified_base (693)..(693): N1-methylpseudouracil
  modified_base (695)..(696): N1-methylpseudouracil
  modified_base (698)..(698): N1-methylpseudouracil
  modified_base (700)..(702): N1-methylpseudouracil
  modified_base (708)..(708): N1-methylpseudouracil
  modified_base (713)..(714): N1-methylpseudouracil
  modified_base (716)..(716): N1-methylpseudouracil
  modified_base (718)..(718): N1-methylpseudouracil
  modified_base (720)..(720): N1-methylpseudouracil
  modified_base (723)..(723): N1-methylpseudouracil
  modified_base (726)..(726): N1-methylpseudouracil
  modified_base (735)..(735): N1-methylpseudouracil
  modified_base (741)..(741): N1-methylpseudouracil
  modified_base (747)..(747): N1-methylpseudouracil
  modified_base (750)..(750): N1-methylpseudouracil
  modified_base (752)..(752): N1-methylpseudouracil
  modified_base (759)..(759): N1-methylpseudouracil
  modified_base (776)..(776): N1-methylpseudouracil
  modified_base (779)..(779): N1-methylpseudouracil
  modified_base (790)..(790): N1-methylpseudouracil
  modified_base (794)..(794): N1-methylpseudouracil
  modified_base (798)..(798): N1-methylpseudouracil
  modified_base (804)..(804): N1-methylpseudouracil
  modified_base (814)..(814): N1-methylpseudouracil
  modified_base (818)..(819): N1-methylpseudouracil
  modified_base (823)..(823): N1-methylpseudouracil
  modified_base (828)..(828): N1-methylpseudouracil
  modified_base (831)..(831): N1-methylpseudouracil
  modified_base (840)..(840): N1-methylpseudouracil
  modified_base (847)..(847): N1-methylpseudouracil
  modified_base (849)..(852): N1-methylpseudouracil
  modified_base (858)..(858): N1-methylpseudouracil
  modified_base (864)..(864): N1-methylpseudouracil
  modified_base (870)..(870): N1-methylpseudouracil
  modified_base (874)..(874): N1-methylpseudouracil
  modified_base (882)..(882): N1-methylpseudouracil
  modified_base (884)..(884): N1-methylpseudouracil
  modified_base (896)..(896): N1-methylpseudouracil
  modified_base (898)..(898): N1-methylpseudouracil
  modified_base (909)..(909): N1-methylpseudouracil
  modified_base (911)..(911): N1-methylpseudouracil
  modified_base (917)..(917): N1-methylpseudouracil
  modified_base (921)..(921): N1-methylpseudouracil
  modified_base (925)..(927): N1-methylpseudouracil
  modified_base (933)..(934): N1-methylpseudouracil
  modified_base (938)..(938): N1-methylpseudouracil
  modified_base (942)..(942): N1-methylpseudouracil
  modified_base (945)..(945): N1-methylpseudouracil
  modified_base (950)..(950): N1-methylpseudouracil
  modified_base (952)..(952): N1-methylpseudouracil
  modified_base (954)..(954): N1-methylpseudouracil
  modified_base (957)..(957): N1-methylpseudouracil
  modified_base (960)..(960): N1-methylpseudouracil
  modified_base (963)..(963): N1-methylpseudouracil
  modified_base (969)..(969): N1-methylpseudouracil
  modified_base (971)..(972): N1-methylpseudouracil
  modified_base (981)..(981): N1-methylpseudouracil
  modified_base (988)..(988): N1-methylpseudouracil
  modified_base (991)..(992): N1-methylpseudouracil
  modified_base (999)..(999): N1-methylpseudouracil
  modified_base (1005)..(1005): N1-methylpseudouracil
  modified_base (1008)..(1008): N1-methylpseudouracil
  modified_base (1010)..(1011): N1-methylpseudouracil
  modified_base (1013)..(1013): N1-methylpseudouracil
  modified_base (1017)..(1017): N1-methylpseudouracil
  modified_base (1020)..(1020): N1-methylpseudouracil
  modified_base (1025)..(1026): N1-methylpseudouracil
  modified_base (1028)..(1028): N1-methylpseudouracil
  modified_base (1041)..(1041): N1-methylpseudouracil
  modified_base (1044)..(1044): N1-methylpseudouracil
  modified_base (1050)..(1050): N1-methylpseudouracil
  modified_base (1057)..(1057): N1-methylpseudouracil
  modified_base (1059)..(1059): N1-methylpseudouracil
  modified_base (1065)..(1065): N1-methylpseudouracil
  modified_base (1068)..(1068): N1-methylpseudouracil
  modified_base (1072)..(1073): N1-methylpseudouracil
  modified_base (1075)..(1076): N1-methylpseudouracil
  modified_base (1080)..(1080): N1-methylpseudouracil
  modified_base (1089)..(1089): N1-methylpseudouracil
  modified_base (1092)..(1092): N1-methylpseudouracil
  modified_base (1102)..(1102): N1-methylpseudouracil
  modified_base (1105)..(1105): N1-methylpseudouracil
  modified_base (1112)..(1112): N1-methylpseudouracil
  modified_base (1116)..(1116): N1-methylpseudouracil
  modified_base (1118)..(1118): N1-methylpseudouracil
  modified_base (1121)..(1121): N1-methylpseudouracil
  modified_base (1124)..(1124): N1-methylpseudouracil
  modified_base (1127)..(1127): N1-methylpseudouracil
  modified_base (1131)..(1131): N1-methylpseudouracil
  modified_base (1136)..(1136): N1-methylpseudouracil
  modified_base (1146)..(1146): N1-methylpseudouracil
  modified_base (1152)..(1152): N1-methylpseudouracil
  modified_base (1159)..(1159): N1-methylpseudouracil
  modified_base (1163)..(1163): N1-methylpseudouracil
  modified_base (1168)..(1168): N1-methylpseudouracil
  modified_base (1172)..(1173): N1-methylpseudouracil
  modified_base (1175)..(1176): N1-methylpseudouracil
  modified_base (1182)..(1183): N1-methylpseudouracil
  modified_base (1189)..(1189): N1-methylpseudouracil
  modified_base (1193)..(1193): N1-methylpseudouracil
  modified_base (1198)..(1198): N1-methylpseudouracil
  modified_base (1208)..(1208): N1-methylpseudouracil
  modified_base (1212)..(1212): N1-methylpseudouracil
  modified_base (1225)..(1225): N1-methylpseudouracil
  modified_base (1228)..(1228): N1-methylpseudouracil
  modified_base (1233)..(1234): N1-methylpseudouracil
  modified_base (1237)..(1237): N1-methylpseudouracil
  modified_base (1245)..(1245): N1-methylpseudouracil
  modified_base (1252)..(1252): N1-methylpseudouracil
  modified_base (1256)..(1256): N1-methylpseudouracil
  modified_base (1261)..(1261): N1-methylpseudouracil
  modified_base (1271)..(1271): N1-methylpseudouracil
  modified_base (1276)..(1276): N1-methylpseudouracil
  modified_base (1279)..(1279): N1-methylpseudouracil
  modified_base (1281)..(1283): N1-methylpseudouracil
  modified_base (1287)..(1287): N1-methylpseudouracil
  modified_base (1292)..(1292): N1-methylpseudouracil
  modified_base (1294)..(1294): N1-methylpseudouracil
  modified_base (1298)..(1298): N1-methylpseudouracil
ggaaanaagagagaaaagaagagnaagaagaaananaagagccaccanggaggagggcangaacgngcnncacgacnncggcanccagncaacccannaccnacaggngaacnancaggacncccaggannggnncanccncgngagcgngancgccgancnaagaaacgcanncnacgngnngnncccaancnggnnccaccngcaggaagcngngggcancaagcnccnnngggnggccgngannggcgannggcngaaccnggngnncaagngganncncnncggncagcggccnnacnggngggngnngganaccgacnannacagnaacaccncngnnccgcngancaagcagnncccngncaccngcgaaaccggaccnggcnccccnnccggccacgccangggcaccgccggcgngnacnacgnganggngacanccacccngagcancnnccagggcaagancaagccnaccnacaganncaggngccngaacgnnancnngnggcnggganncngggcagngcagcncaacgngngccngagcaggancnancncgcagcccacnncccacancaggncgnggccggcgnacngagcggaancgccgnggcngaaaccnncagccananccacagcancnacaangccncccngaagaagnacnnccnnannacanncnnccngnncncnnncgccancggcnncnancngcngcnaaagggccngggcgncgaccnccngnggacccnggagaaggcccagaganggngcgaacaaccngagngggngcacancgacaccacnccgnncgcnagccngcncaagaaccngggcacncnnnncggccngggccnggcccncaanagcagcangnaccgggagagcngnaagggaaagcngnccaagnggcncccnnncagacnnagcngcancgnggcgncncncgngcngcngcacgngnncgacagccngaagccnccnncgcaggnggagcnggngnncnacgngcngagcnncngcaagagcgcggnggngccacnggcaagngncagcgngancccnnanngccnggcccaggngcngggccagccncanaagaagncccngnganaanagnccanaaagnaggaaacacnacagcnggagccncggnggccnagcnncnngccccnngggccnccanaaagnaggaaacacnacanccccccagccccnccnccccnnccngcacccgnacccccnccanaaagnaggaaacacnacagnggncnnngaanaaagncngagngggcggc

SEQ ID NO: 6 - RNA, 1342 nt, Artificial Sequence
  source: /note=“Description of Artificial Sequence: Synthetic polynucleotide”
gggaaauaagagagaaaagaagaguaagaagaaauauaagagccaccauggaggagggcaugaacgugcuucacgacuucggcauccagucaacccauuaccuacaggugaacuaucaggacucccaggauugguucauccucgugagcgugaucgccgaucuaagaaacgcauucuacguguuguucccaaucugguuccaccugcaggaagcugugggcaucaagcuccuuuggguggccgugauuggcgauuggcugaaccugguguucaaguggauucucuucggucagcggccuuacugguggguguuggauaccgacuauuacaguaacaccucuguuccgcugaucaagcaguucccugucaccugcgaaaccggaccuggcuccccuuccggccacgccaugggcaccgccggcguguacuacgugauggugacauccacccugagcaucuuccagggcaagaucaagccuaccuacagauucaggugccugaacguuaucuuguggcugggauucugggcagugcagcucaacgugugccugagcaggaucuaucucgcagcccacuucccacaucaggucguggccggcguacugagcggaaucgccguggcugaaaccuucagccauauccacagcaucuacaaugccucccugaagaaguacuuccuuauuacauucuuccuguucucuuucgccaucggcuucuaucugcugcuaaagggccugggcgucgaccuccuguggacccuggagaaggcccagagauggugcgaacaaccugagugggugcacaucgacaccacuccguucgcuagccugcucaagaaccugggcacucuuuucggccugggccuggcccucaauagcagcauguaccgggagagcuguaagggaaagcuguccaaguggcucccuuucagacuuagcugcaucguggcgucucucgugcugcugcacguguucgacagccugaagccuccuucgcagguggagcugguguucuacgugcugagcuucugcaagagcgcgguggugccacuggcaagugucagcgugaucccuuauugccuggcccaggugcugggccagccucauaagaagucccugugauaauaggcuggagccucgguggccuagcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 7-49 - skipped (43 entries)

SEQ ID NO: 50 - RNA, 75 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgcc

SEQ ID NO: 51 - RNA, 203 nt, Artificial Sequence
  source: /note=“Description of Artificial Sequence: Synthetic polynucleotide”
ggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 52 - RNA, 76 nt, Artificial Sequence
  source: /note=“Description of Artificial Sequence: Synthetic oligonucleotide”
ggaaauaagagagaaaagaagaguaagaagaaauauaagaucucccugagcuucagggagccccggcgccgccacc

SEQ ID NO: 53 - RNA, 89 nt, Artificial Sequence
  source: /note=“Description of Artificial Sequence: Synthetic oligonucleotide”
ggaaaccccccacccccguaagagagaaaagaagaguaagaagaaauauaagaucucccugagcuucagggagccccggcgccgccacc

SEQ ID NO: 54 - RNA, 59 nt, Artificial Sequence
  source: /note=“Description of Artificial Sequence: Synthetic oligonucleotide”
ggagaacuuccgcuuccguuggcgcaagcgcuuucauuuuuucugcuaccgugacuaag

SEQ ID NO: 55 - RNA, 46 nt, Artificial Sequence
  source: /note=“Description of Artificial Sequence: Synthetic oligonucleotide”
ggaaauaagagagaaaagaagaguaagaagaaauauaagagccacc

SEQ ID NO: 56 - RNA, 56 nt, Artificial Sequence
  source: /note=“Description of Artificial Sequence: Synthetic oligonucleotide”
ggaaauaagagagaaaagaagaguaagaagaaauauaagaccccggcgccgccacc

SEQ ID NO: 57 - RNA, 81 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgccgccgcc

SEQ ID NO: 58 - RNA, 81 nt, Artificial Sequence
  source: /note=“Description of Artificial Sequence: Synthetic oligonucleotide”
ggaaaucgcaaaauuuucuuuucgcguuagauuucuuuuaguuuucuuucaacuagcaagcuuuuuguucucgccgccgcc

SEQ ID NO: 59 - RNA, 83 nt, Artificial Sequence
  source: /note=“Description of Artificial Sequence: Synthetic oligonucleotide”
  misc_feature (14)..(18): /note=“This region may encompass 0-5 nucleotides”
  variation (19)..(19): /replace=“ ”
  variation (22)..(22): /replace=“ ”
  misc_feature (23)..(27): /note=“This region may encompass 0-5 nucleotides”
  variation (81)..(83): /replace=“ ”
  misc_feature (1)..(83): /note=“Variant nucleotides given in the sequence have no preference with respect to those in the annotations for variant positions”
  source: /note=“See specification as filed for detailed description of substitutions and preferred embodiments”
ggaaaucgcaaaauuuuugcucuuuuucgcguuagauuucuuuuaguuuucuykcaacuagcaagcuuuuuguucucgccrcc

SEQ ID NO: 60 - RNA, 79 nt, Artificial Sequence
  source: /note=“Description of Artificial Sequence: Synthetic oligonucleotide”
ggaaaauuuuagccuggaacguuagauaacuguccuguugucuuuauauacuugguccccaaguaguuugucuuccaaa

SEQ ID NO: 61 - RNA, 79 nt, Artificial Sequence
  source: /note=“Description of Artificial Sequence: Synthetic oligonucleotide”
ggaaacuuuauuuaguguuacuuuauuuucuguuuauuuguguuucuucaguggguuuguucuaauuuccuuggccgcc

SEQ ID NO: 62 - RNA, 79 nt, Artificial Sequence
  source: /note=“Description of Artificial Sequence: Synthetic oligonucleotide”
ggaaaaucuguauuagguuggcguguucuuuggucgguuguuaguauuguuguugauucguuuguggucgguugccgcc

SEQ ID NO: 63 - RNA, 79 nt, Artificial Sequence
  source: /note=“Description of Artificial Sequence: Synthetic oligonucleotide”
ggaaaauuauuaacaucuugguauucucgauaaccauucguuggauuuuauuguauucguaguuuggguuccugccgcc

SEQ ID NO: 64 - RNA, 79 nt, Artificial Sequence
  source: /note=“Description of Artificial Sequence: Synthetic oligonucleotide”
ggaaauuauuauuauuucuagcuacaauuuaucauuguauuauuuuagcuauucaucauuauuuacuuggugaucaaca

SEQ ID NO: 65 - RNA, 79 nt, Artificial Sequence
  source: /note=“Description of Artificial Sequence: Synthetic oligonucleotide”
ggaaauagguuguuaaccaaguucaagccuaauaagcuuggauucuggugacuugcuucaccguuggcgggcaccgauc

SEQ ID NO: 66 - RNA, 74 nt, Artificial Sequence
  source: /note=“Description of Artificial Sequence: Synthetic oligonucleotide”
ggaaaucguagagagucguacuuaguacauaucgacuaucgguggacaccaucaagauuauaaaccaggccaga

SEQ ID NO: 67 - RNA, 79 nt, Artificial Sequence
  source: /note=“Description of Artificial Sequence: Synthetic oligonucleotide”
ggaaacccgcccaagcgaccccaacauaucagcaguugcccaaucccaacucccaacacaauccccaagcaacgccgcc

SEQ ID NO: 68 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguuguuucc

SEQ ID NO: 69 - RNA, 79 nt, Artificial Sequence
  source: /note=“Description of Artificial Sequence: Synthetic oligonucleotide”
ggaaacuaaucgaaauaaaagagccccguacucuuuuauuucuauuagguuaggagccuuagcauuuguaucuuaggua

SEQ ID NO: 70 - RNA, 79 nt, Artificial Sequence
  source: /note=“Description of Artificial Sequence: Synthetic oligonucleotide”
ggaaaugugauuuccagcaacuucuuuugaauauauugaauuccuaauucaaagcgaacaaaucuacaagccauauacc

SEQ ID NO: 71 - RNA, 74 nt, Artificial Sequence
  source: /note=“Description of Artificial Sequence: Synthetic oligonucleotide”
ggaaaucguagagagucguacuuacguggucgccauugcauagcgcgcgaaagcaacaggaacaagaacgcgcc

SEQ ID NO: 72 - RNA, 74 nt, Artificial Sequence
  source: /note=“Description of Artificial Sequence: Synthetic oligonucleotide”
ggaaaucguagagagucguacuuagaauaaacagagucgggucgacuugucucugauacuacgacgucacaauc

SEQ ID NO: 73 - RNA, 79 nt, Artificial Sequence
  source: /note=“Description of Artificial Sequence: Synthetic oligonucleotide”
ggaaaauuugccuucggaguugcguauccugaacugcccagccuccugauauacaacuguuccgcuuauucgggccgcc

SEQ ID NO: 74 - RNA, 79 nt, Artificial Sequence
  source: /note=“Description of Artificial Sequence: Synthetic oligonucleotide”
ggaaaucugagcaggaauccuuugugcauugaagacuuuagauuccucucugcgguagacgugcacuuauaaguauuug

SEQ ID NO: 75 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguugccacc

SEQ ID NO: 76 - RNA, 104 nt, Artificial Sequence
  source: /note=“Description of Artificial Sequence: Synthetic polynucleotide”
ggaaauuuuuuuuugauauuauaagaguuuuuuuuugauauuaagaaaauuuuuuuuugauauuagaagaguaagaagaaauauaagaccccggcgccgccacc

SEQ ID NO: 77 - RNA, 57 nt, Artificial Sequence
  source: /note=“Description of Artificial Sequence: Synthetic oligonucleotide”
ggaaauaagagagaaaagaagaguaagaagaaauauaagagccaaaaaaaaaaaacc

SEQ ID NO: 78 - RNA, 76 nt, Artificial Sequence
  source: /note=“Description of Artificial Sequence: Synthetic oligonucleotide”
ggaaaucucccugagcuucagggaguaagagagaaaagaagaguaagaagaaauauaagaccccggcgccgccacc

SEQ ID NO: 79 - RNA, 6 nt, Artificial Sequence
  source: /note=“Description of Artificial Sequence: Synthetic oligonucleotide”
gccrcc

SEQ ID NO: 80-99 - skipped (20 entries)

SEQ ID NO: 100 - RNA, 119 nt, Artificial Sequence
  source: /note=“Description of Artificial Sequence: Synthetic polynucleotide”
uaauaguaagcuggagccuccugagagaccugugugaacuauugagaagaucggaacagcuccuuacucugaggaaguugguacccccguggucuuugaauaaagucugagugggcggc

SEQ ID NO: 101 - RNA, 119 nt, Artificial Sequence
  source: /note=“Description of Artificial Sequence: Synthetic polynucleotide”
uaauaguaagcuggagccucacucuccucuccaucccguauccaggcugugaauuuuucaaggaauauaaagaucgggauguacccccguggucuuugaauaaagucugagugggcggc

SEQ ID NO: 102 - RNA, 119 nt, Artificial Sequence
  source: /note=“Description of Artificial Sequence: Synthetic polynucleotide”
ugauaguaagcuggagccucuagugacggcaacagggcuugguuuuuccuuguugugaaaucgacaucucugaagacaggguacccccguggucuuugaauaaagucugagugggcggc

SEQ ID NO: 103 - RNA, 119 nt, Artificial Sequence
  source: /note=“Description of Artificial Sequence: Synthetic polynucleotide”
ugauaguaagcuggagccuccuuccaucuagucacaaagacuccuucguccccaguugccgucuaggauugggccucccaguacccccguggucuuugaauaaagucugagugggcggc

SEQ ID NO: 104 - RNA, 119 nt, Artificial Sequence
  source: /note=“Description of Artificial Sequence: Synthetic polynucleotide”
ugauaguaagcuggagccucccauaacaugacauaucuggauuuugugcuuagaaccuuaaauuggaagcauucuuaauuguacccccguggucuuugaauaaagucugagugggcggc

SEQ ID NO: 105 - RNA, 119 nt, Artificial Sequence
  source: /note=“Description of Artificial Sequence: Synthetic polynucleotide”
uaauaguaagcuggagccuccggaaaacuaaaauagagauauuucaagauuuuauaauuuucaaagaccuuugaaauauuguacccccguggucuuugaauaaagucugagugggcggc

SEQ ID NO: 106 - RNA, 119 nt, Artificial Sequence
  source: /note=“Description of Artificial Sequence: Synthetic polynucleotide”
uaauaguaagcuggagccucuacacauugcuucuaguuggcagaaauaauugauuaaaagaccagaaacugugauaacugguacccccguggucuuuaaauaaagucuaagugggcggc

SEQ ID NO: 107 - RNA, 119 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 108 - RNA, 119 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 109 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 110 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 111 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 112 - RNA, 141 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 113 - RNA, 164 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 114 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 115 - RNA, 60 nt, Artificial Sequence
  source: /note=“Description of Artificial Sequence: Synthetic oligonucleotide”
cugagagaccugugugaacuauugagaagaucggaacagcuccuuacucugaggaaguug

SEQ ID NO: 116 - RNA, 119 nt, Artificial Sequence
  source: /note=“Description of Artificial Sequence: Synthetic polynucleotide”
ugauaauaggcuggagccucucacacaccucugccccuugggccucccacucccauggcucugggcgguccagaaggagcguacccccguggucuuugaauaaagucugagugggcggc

SEQ ID NO: 117 - RNA, 109 nt, Artificial Sequence
  source: /note=“Description of Artificial Sequence: Synthetic polynucleotide”
ugauaauaggcuggagccuccaccgcguuauccguuccucguaggcugguccuggggaacgggucggcggguacccccguggucuuugaauaaagucugagugggcggc

SEQ ID NO: 118 - RNA, 257 nt, Artificial Sequence
  source: /note=“Description of Artificial Sequence: Synthetic polynucleotide”
ugauaauaggcuggagccucugcccggcaacggccaggucugugccaaguguuugcugacgcaacccccacuggcuggggcuuggucaugggccaucagcgcgugcguggaaccuuuucggcuccucugccgauccauacugcggaacuccuagccgcuuguuuugcucgcagcaggucuggagcaaacauuaucgggacugauaacucuguuguccuguacccccguggucuuugaauaaagucugagugggcggc

SEQ ID NO: 119 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuucuucuuuucuuuuuuuucuuuuuuuuuuuucuuucuuuuuuucuuuuuuuuucuuuucuuuuuucuuuuuuuuuuuuuuuuccguggucuuugaauaaagucugagugggcggc

SEQ ID NO: 120 - RNA, 219 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccguggucuuugaauaaagucugagugggcggc

SEQ ID NO: 121 - RNA, 119 nt, Artificial Sequence
  source: /note=“Description of Artificial Sequence: Synthetic polynucleotide”
uaggguuaagcuggagccucgguggccuagcuucuugccccuugggccuccccccagccccuccuccccuuccugcacccguacccccguggucuuugaauaaagucugagugggcggc

SEQ ID NO: 122 - RNA, 119 nt, Artificial Sequence
  source: /note=“Description of Artificial Sequence: Synthetic polynucleotide”
uaaagcuccgcuggagccucgguggccuagcuucuugccccuugggccuccccccagccccuccuccccuuccugcacccguacccccguggucuuugaauaaagucugagugggcggc

SEQ ID NO: 123 - RNA, 119 nt, Artificial Sequence
  source: /note=“Description of Artificial Sequence: Synthetic polynucleotide”
uaagccccugcuggagccucgguggccuagcuucuugccccuugggccuccccccagccccuccuccccuuccugcacccguacccccguggucuuugaauaaagucugagugggcggc

SEQ ID NO: 124 - RNA, 119 nt, Artificial Sequence
  source: /note=“Description of Artificial Sequence: Synthetic polynucleotide”
uaagcacccgcuggagccucgguggccuagcuucuugccccuugggccuccccccagccccuccuccccuuccugcacccguacccccguggucuuugaauaaagucugagugggcggc

SEQ ID NO: 125 - RNA, 119 nt, Artificial Sequence
  source: /note=“Description of Artificial Sequence: Synthetic polynucleotide”
uaagccccuccgggggccucgguggccuagcuucuugccccuugggccuccccccagccccuccuccccuuccugcacccguacccccguggucuuugaauaaagucugagugggcggc

SEQ ID NO: 126 - RNA, 119 nt, Artificial Sequence
  source: /note=“Description of Artificial Sequence: Synthetic polynucleotide”
uaaggcuaagcuggagccucgguggccuagcuucuugccccuugggccuccccccagccccuccuccccuuccugcacccguacccccguggucuuugaauaaagucugagugggcggc

SEQ ID NO: 127 - RNA, 119 nt, Artificial Sequence
  source: /note=“Description of Artificial Sequence: Synthetic polynucleotide”
uaagucuccgcuggagccucgguggccuagcuucuugccccuugggccuccccccagccccuccuccccuuccugcacccguacccccguggucuuugaauaaagucugagugggcggc

SEQ ID NO: 128 - RNA, 119 nt, Artificial Sequence
  source: /note=“Description of Artificial Sequence: Synthetic polynucleotide”
uaaagcuaagcuggagccucgguggccuagcuucuugccccuugggccuccccccagccccuccuccccuuccugcacccguacccccguggucuuugaauaaagucugagugggcggc

SEQ ID NO: 129 - RNA, 119 nt, Artificial Sequence
  source: /note=“Description of Artificial Sequence: Synthetic polynucleotide”
uaagucuaagcuggagccucgguggccuagcuucuugccccuugggccuccccccagccccuccuccccuuccugcacccguacccccguggucuuugaauaaagucugagugggcggc

SEQ ID NO: 130 - RNA, 185 nt, Artificial Sequence
  source: /note=“Description of Artificial Sequence: Synthetic polynucleotide”
uaaagcuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 131 - RNA, 188 nt, Artificial Sequence
  source: /note=“Description of Artificial Sequence: Synthetic polynucleotide”
uaagccccuccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 132 - RNA, 188 nt, Artificial Sequence
  source: /note=“Description of Artificial Sequence: Synthetic polynucleotide”
uaaagcuccccgggguccauaaaguaggaaacacuacagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 133 - RNA, 185 nt, Artificial Sequence
  source: /note=“Description of Artificial Sequence: Synthetic polynucleotide”
uaagucuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 134 - RNA, 139 nt, Artificial Sequence
  source: /note=“Description of Artificial Sequence: Synthetic polynucleotide”
uaaagcgcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 135 - RNA, 194 nt, Artificial Sequence
  source: /note=“Description of Artificial Sequence: Synthetic polynucleotide”
uaaagcuccccgggguccauaaaguaggaaacacuacagcuggagccuccugagagaccugugugaacuauugagaagaucggaacagcuccuuacucugaggaaguuguccauaaaguaggaaacacuacaguacccccuccauaaaguaggaaacacuacaguggucuuugaauaaagucugagugggcggc

SEQ ID NO: 136 - RNA, 241 nt, Artificial Sequence
  source: /note=“Description of Artificial Sequence: Synthetic polynucleotide”
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 137-151 - skipped (15 entries)

SEQ ID NO: 152 - RNA, 22 nt, Artificial Sequence
  source: /note=“Description of Artificial Sequence: Synthetic oligonucleotide”
cgcauuauuacucacgguacga

SEQ ID NO: 153-173 - skipped (21 entries)

SEQ ID NO: 174 - RNA, 23 nt, Artificial Sequence
  source: /note=“Description of Artificial Sequence: Synthetic oligonucleotide”
uccauaaaguaggaaacacuaca

SEQ ID NO: 175-194 - skipped (20 entries)

SEQ ID NO: 195 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - RNA, 120 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 197-208 - skipped (12 entries)

SEQ ID NO: 209 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (21)..(21): Inverted deoxythymidine
aaaaaaaaaaaaaaaaaaaan

SEQ ID NO: 210 - skipped

SEQ ID NO: 211 - DNA, 126 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (126)..(126): Inverted deoxythymidine
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuagaaaaaaaaaaaaaaaaaaaan

SEQ ID NO: 212 - RNA, 22 nt, Artificial Sequence
  source: /note=“Description of Artificial Sequence: Synthetic oligonucleotide”
caaacaccauugucacacucca

SEQ ID NO: 213 - skipped

SEQ ID NO: 214 - RNA, 9 nt, Artificial Sequence
  source: /note=“Description of Artificial Sequence: Synthetic oligonucleotide”
ccrccaugg